(19) United States
(12) Patent Application Publication
(10) Pub. No.: US 2011/0028458 A1
(43) Pub. Date: Feb. 3, 2011

(54) INHIBITION OF CELL MIGRATION BY A FARNESYLATED DIBENZODIAZEPINONE

(75) Inventors: Borhane Annabi, Brossard (CA); Martha Maria Cajina Herrera, Montreal (CA); Henriette Gourdeau, Montreal (CA)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: THALLION PHARMACEUTICALS INC., Montreal, QC (CA)

(21) Appl. No.: 12/937,101

(22) PCT Filed: Apr. 9, 2009
(86) PCT No.: PCT/CA09/00485

§ 371 Date: Oct. 8, 2010

Related U.S. Application Data
(60) Provisional application No. 61/044,288, filed on Apr. 11, 2008.

(30) Foreign Application Priority Data
Oct. 24, 2008 (US) 12/258102

(51) Int. Cl. A61K 31/551; C12N 5/09; A61P 35/00

(52) U.S. Cl. 514/220; 435/375

(57) ABSTRACT

The invention relates to the discovery that dibenzodiazepinone analogues have cell migration inhibiting activities on neoplastic and endothelial cells. The migration of neoplastic cells from various tumor types, such as a glioma tumor that may comprise an EGF and/or PTEN mutation, or a Ras-, Raf, or EGFR-mediated tumor, may be inhibited when contacted by the dibenzodiazepinone analogues of the present invention. The invention includes methods for inhibiting migration of a cell in a subject, by contacting a cell with a dibenzodiazepinone analogue of the present invention.

### FIELD OF THE INVENTION

The present invention relates to dibenzodiazepinone analogues, including a naturally produced farnesylated dibenzodiazepinone referred to herein as Compound 1, and to chemical derivatives of the analogues, as well as to pharmaceutically acceptable salts, esters, solvates and prodrugs of the analogues and derivatives, and to methods for obtaining these compounds. One method of obtaining Compound 1 is by cultivation of a strain of a Micromonospora sp., e.g, 046-ECO11 or [S01]046. One method of obtaining the derivatives involves post-biosynthesis chemical modification of Compound 1. The present invention further relates to the use of dibenzodiazepinone analogues, including Compound 1, and their pharmaceutically acceptable salts, esters, solvates and prodrugs as pharmaceuticals, in particular to their use as inhibitors of cancer cell growth and migration as well as for treating acute and chronic inflammation.

The invention further relates to the discovery that the dibenzodiazepinone analogues, including Compound 1, can inhibit migration of neoplastic cells that are driven by expression of RAS or mutated RAS, and/or which are neoplastic cells of EGF-mediated tumors and/or a Raf kinase-mediated tumors and/or PI3K/AKT-mediated tumors. As well, the present invention further relates to the discovery that the dibenzodiazepinone analogues, including Compound 1, have cell migration inhibiting activities on endothelial cells, and furthermore, that the migration of the endothelial cells can be inhibited by the dibenzodiazepinone analogues, including Compound 1, when the migration of these cells is induced in response to a chemotactic stimulant such as a presence of one or more growth factors. The present invention thus further includes methods for inhibiting migration of a cell, which may be a neoplastic or endothelial cell, by contacting a cell with a dibenzodiazepoinone analogue, including Compound 1. Such contact may occur in an in vitro or in vivo environment. The present invention further includes methods for inhibiting migration of a cell in a subject, comprising administering an effective amount of a farnesylated dibenzodiazepinone analogue, including Compound 1, to the subject to thereby inhibit migration of a cell or metastasis of a tumor to the subject.

### BACKGROUND OF THE INVENTION

### Part A

The euactinomycetes are a subset of a large and complex group of

Gram-positive bacteria known as actinomycetes. Over the past few decades these organisms, which are abundant in soil, have generated significant commercial and scientific interest as a result of the large number of therapeutically useful compounds, particularly antibiotics, produced as secondary metabolites. The intensive search for strains able to produce new antibiotics has led to the identification of hundreds of new species.

Many of the euactinomycetes, particularly Streptomyces and the closely related Saccharopolyspora genera, have been extensively studied. Both of these genera produce a notable diversity of biologically active metabolites. Because of the commercial significance of these compounds, much is known about the genetics and physiology of these organisms.

Another representative genus of euactinomycetes, Micromonospora, has also generated commercial interest. For example, U.S. Pat. No. 5,541,181 (Ohkuma et al., 1996) discloses a dibenzodiazepinone compound, specifically 5-farnesyl-4,7,9-trihydroxy-dibenzodiazepin-11-one (named “BU-4664L”), produced by a known euactinomycetes strain, Micromonospora sp. M990-6 (ATCC 55378).

TLN-4601 [previously referred to as ECO-4601] (4,6,8-trihydroxy-10-(3,7,11-trimethyldodeca-2,6,10-trienyl)-5,10-dihydrodibenzo[b, e][1,4]diazepin-11-one) is a farnesylated dibenzodiazepinone (MW 462.58) (see Bachmann et al (2004) U.S. Pat. No. 7,101,872 and Canadian Patent No. 2,466,340) is one of the natural compounds identified using DECIPHER® to analyze actinomycete gene loci encoding pathways leading to bioactive compounds (see Farnet and Zazopoulos (2005) in Natural Products: Drug Discovery and Therapeutic Medicine at pp. 95-106; McAlpine et al. (2005) Journal of Natural Products vol. 68, pp. 493-496; Zazopoulos et al. (2003) Nature BioTechnology, vol. 21, pp. 187-190). The compound was also isolated and characterized by Wyeth Laboratories (see Charan et al. (2004) Journal to of Natural Products, vol. 67, pp. 1431-1434). Initial in vitro assessment by the U.S. National Cancer Institute (NCI) showed that TLN-4601 had broad cytotoxic activity in the low micromolar range inhibiting the growth of hematological and solid tumor cell lines, and thus a good candidate for clinical studies against brain and other solid tumors.

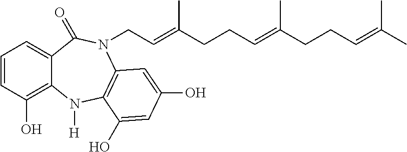

### TLN-4601 (Compound 1 of the Present Invention)

Part B

The EGFR (ErbB1, HER1) is the prototypic member of the ErbB family of receptor tyrosine kinases, which further consists of ErbB2-4 (HER2-4) (Hynes and Lane (2005) Nature Reviews Cancer, vol. 5, pp. 341-354). Two of the main pathways activated by the epidermal growth factor (ERBB) receptors are the mitogen activated protein kinase (MAPK) and the phosphatidylinositol 3-kinase (PI3K)/AKT pathways (Yaren and Sliwkowski (2001) Nature Rev Mo. Cell Biol vol. 2, pp. 127-137).

The RAS-MAPK signaling pathway is one of the signaling pathways involved in control of cell growth, differentiation and survival. This signaling pathway has long been viewed as an attractive pathway for anticancer therapies, based on its central role in regulating the growth and survival of cells from a broad spectrum of human tumors, and mutations in components of this signaling pathway underlie tumour initiation in mammal cells (Sebolt-Leopold et al. (2004) Nature Reviews Cancer, vol. 4, pp. 937-947).

The RAS-MAPK signaling pathway is activated by a variety of extracellular signals (hormones and growth factors). Moreover, mutations in components of this signaling pathway, resulting in constitutive activation, underlie tumor initiation in mammalian cells. For example, growth factor receptors, such as epidermal growth factor receptor (EGFR), are subject to amplifications and mutations in many cancers, accounting for up to 25% of non-small cell lung cancers and 60% of glioblastomas. BRaf is also frequently mutated, particularly in melanomas (approximately 70% of cases) and colon carcinomas (approximately 15% of cases). Moreover, ras is the most frequently mutated oncogene, occurring in approximately 30% of all human cancers. The frequency and type of mutated ras genes (H-ras, K-ras or N-ras) varies widely depending on the tumor type. K-ras is, however, the most frequently mutated gene, with the highest incidence detected in pancreatic cancer (approximately 90%) and colorectal cancer (approximately 45%).

The PI3K/AKT pathway regulates several critical cellular functions including cell cycle progression, migration, invasion, and survival as well as angiogenesis (Katso et al. (2001) Annu Rev Cell Dev Biol, vol.17, pp. 615-675). In addition, the activated PI3K/AKT provides major survival functions to glioblastoma multiform cells and many other cancer cells. Furthermore, the ectopic expression of AKT induces cell survival and malignant transformation, whereas the inhibition of AKT activity stimulates apoptosis.

There is a need to develop novel compounds and methods of treatment for cancer and other diseases in humans. The present invention addresses these problems by providing novel uses and methods of using a farnesylated dibenzodiazepinone, including Compound 1, for therapeutic inhibition of neoplastic and/or endothelial cell migration.

### SUMMARY OF THE INVENTION

The present invention is directed to methods for inhibiting migration of a cell comprising contacting a cell with an effective amount of a compound of Formula I or a pharmaceutically acceptable salt, ester or solvate thereof. In one embodiment, the compound is a compound selected from Compounds 1 to 100, preferably Compound 1. In a further embodiment, the cell is contacted either in vitro or in vivo, and in a still further embodiment, the cell is a neoplastic cell or an endothelial cell. In a still further embodiment, the migration that is inhibited by contact with the compound of Formula I is a chemotactic migration, and in a still further embodiment, the chemotactic migration is induced by activation of the epidermal growth factor receptor pathways, comprising the Ras-MAPK signaling and PI3K/AKT signaling pathways in the cell. In still further embodiments, the neoplastic cell in which migration is inhibited is a cell of a glioma tumor or glioblastoma multiform tumor comprising an EGF receptor mutation, a PTEN mutation, or both an EGF receptor mutation and a PTEN mutation. In a still further embodiment, the EGF receptor mutation is an EGFRvIII mutation.

The invention further encompasses methods for inhibiting migration of a cell in a subject comprising administering an effective amount of a compound of Formula I or a pharmaceutically acceptable salt, ester or solvate thereof to a subject. In one embodiment, the compound is a compound selected from Compounds 1 to 100, preferably Compound 1. In a further embodiment, the cell is a neoplastic cell or an endothelial cell. In a still further embodiment, the migration that is inhibited by contact with the compound of Formula I is a chemotactic migration, and in a still further embodiment, the chemotactic migration is induced by activation of the epidermal growth factor receptor pathways, comprising the Ras-MAPK and/or PI3K/AKT signaling pathways in the cell. In still further embodiments, the neoplastic cell in which the migration is inhibited is a cell of a glioma tumor or glioblastoma multiform tumor comprising an EGF receptor mutation, a PTEN mutation, or both an EGF receptor mutation and a PTEN mutation. In a still further embodiment, the EGF receptor mutation is an EGFRvIII mutation.

### BRIEF DESCRIPTION OF THE FIGURES

FIG. 1: shows the in vitro anti-inflammatory activity of Compound 1. Graph shows percent inhibition of 5-lipoxygenase activity plotted against the Log μM concentration of Compound 1 (“ECO-04601 “) and NDGA. Graph shows the EC₅₀ of Compound 1 to be 0.93 μM.

FIG. 2: shows the pharmokinetic profiles of Compounds 1 and 2 in CD-1 mice following 30 mg/kg intravenous (IV) and intraperitoneal (IP) administrations.

FIG. 3A-B: shows in A. schatchard plot analysis of rat heart mitochondrial membrane using [³H]PK11195 as the specific ligand, and in B. binding displacement of [³H]PK11195 with Compound 1 (“ECO-4601”).

FIG. 4A-B: shows in A and B. In vivo PET images from rat brains before (A) and after (B) administration of TLN-4601, and in C. Bar graph plot results from a competitive binding study utilizing data from n=6 rats and showing a binding potential of ¹¹C-(R)-11195 before and after administration of TLN-4601.

FIG. 5: shows a bar graph plot of TLN-4601 concentrations in plasma and selected tissues obtained from n=6 rats treated CIV with TLN-4601 for 60 min.

FIG. 6A-B: shows in A. Western blot analysis of human breast MCF7 and MDA-MB-231 tumor cells extracts exposed to 10 uM of TLN-4601 for different times as indicated and probed with p-Raf-1, Raf-1, p-ERK 1/2 and ERK 1/2 specific antibodies (GAPDH was used as a loading control), and in B. Western blot analysis of human glioma U87 MG and human prostate PC3 tumor cells extracts exposed to 10 uM of TLN-4601 for different times as indicated and probed with p-Raf-1, Raf-1, p-ERK 1/2 and ERK 1/2 specific antibodies (GAPDH was used as a loading control).

FIG. 7: shows Pull-down and Western blot analyses of human breast MCF7 tumor cells extracts exposed to varying concentrations of TLN-4601 for 18 h. RAS was immunodetected in the pull-down fraction and total fraction using a pan-RAS antibody.

FIG. 8: shows the results of an ERK phosphorylation ELISA assay, where “4601” is Compound 1, “4625” is Compound 97, “4657” is Compound 99 and “4687” is Compound 100.

FIG. 9: shows cell migration assay results from human glioma cell lines (U87 MG parental; U87 MG bearing an amplified copy number of wild-type EGFR; U87 MG bearing a mutated EGFR (EGFRvIII)), wherein the cell lines pretreated (versus non pre-treated control) with TLN-4601 and thereafter assayed for their migration capacity either in an absence or presence of EGF.

FIG. 10: shows results from Western blot analyses for levels of members various proteins of the Ras-MAPK signaling pathway in U87 MG glioma cells, parental and bearing either wild-type (amplified copy number) or mutated (EGFRvIII) epidermal growth factor receptor, the cells having been either pre-treated (versus non pre-treated control) with TLN-4601 and thereafter assayed for their migration capacity either in an absence or presence of EGF.

FIG. 11: shows results from Western blot analyses to assay for a reduction in AKT signaling in U87 glioma cells, parental and bearing either wild-type (amplified copy number) or mutated (EGFRvIII) epidermal growth factor receptor. Cells, treated or not with TLN-4601, were harvested and subjected to Western blot analysis. Bad total and phosphorylation levels were evaluated as readout of AKT activity.

FIG. 12: shows in A. results from measurements of caspase-3 levels in U87 glioma cells (U87 parental; U87 bearing an amplified copy number of EGFR wild type; U87 bearing a mutated EGFR (EGFRvIII)) treated with various concentrations of TLN-4601; and, in B., Western blot analyses to assay for cleavage of PARP in U87 glioma cells (U87 parental; U87 bearing an amplified copy number of EGFR wild type; U87 bearing a mutated EGFR (EGFRvIII)) at various time points after treatment with different concentrations of TLN-4601.

FIG. 13: shows in A. results from a cell migration assay of human brain microvascular endothelial cells pre-treated with 5 μM TLN-4601 (versus untreated control) for 18 hours and thereafter induced to migrate in the presence or absence of brain tumor-derived growth factors; and, in B., a bar graph showing the percentage of cell migration in TLN-4601 pre-treated versus non pre-treated control cells±brain-tumor derived growth factors (human U87 MG conditioned media).

FIG. 14: graph showing levels of caspase-3 induction in U87 glioma cells versus human brain microvascular endothelial cells after treatment with various concentrations of TLN-4601 (expressed as fold induction over untreated cells).

FIG. 15: micrographs showing a reduction in tubulogenesis (capillary-like structure formation) of human brain microvascular endothelial cells after treatment with varying contrations of TLN-4601.

FIG. 16: shows in A. results from Western blot analyses of human brain microvascular endothelial cells (untreated control cells versus cells pretreated with 5 μM TLN-4601) assayed for SIP-mediated phosphorylation of Raf and ERK; and, in B. and C., graphs showing levels of S1P-mediated phosphorylation of Raf and ERK in TLN-4601 treated human brain microvascular endothelial (relative to untreated control cells) at various timepoints after treatment.

FIG. 17: shows in A. results from Western blot analyses of human brain microvascular endothelial cells (untreated control cells versus cells pretreated with 5 μM TLN-4601) assayed for LPA-mediated phosphorylation of Raf and ERK; and, in B. and C., graphs showing levels of LPA-mediated phosphorylation of Raf and ERK in TLN-4601 treated human brain microvascular endothelial (relative to untreated control cells) at various timepoints after treatment.

FIG. 18: shows in A. micrographs of human brain microvascular endothelial cells (untreated control cells versus cells pretreated with 5 μM TLN-4601) stimulated to migrate in response to a presence of a particular chemotactic stimulent (VEGF, bFGF, S1 P, LPA, EGF, NSF, HGF, and LIF); and, in B., a graph showing degree of cell migration in the TLN-4601 pre-treated versus untreated cells in response to the various chemotactic stimuli (as a fold level relative to the untreated control cells); and in C., a numerical presentation of a degree of inhibition of cell migration of the TLN-4601 pre-treated human brain microvascular endothelial cells relative to the untreated control cells in response to the various chemotactic stimuli.

### DETAILED DESCRIPTION OF THE INVENTION

The present invention relates to the discovery that the dibenzodiazepinone analogues, including Compound 1, have cell migration inhibiting activities on neoplastic and endothelial cells. Thus, the invention includes a use of the dibenzodiazepinone analogues, including Compound 1, for inhibiting the migration of neoplastic and andothelial cells, whether in vitro or in vivo, comprising contacting a cell with an effective amount of a compound of Formula I or a pharmaceutically acceptable salt, ester or solvate thereof. In a particular embodiment, the migration that is inhibited by contact with the compound of Formula I is a chemotactic migration, and in a still further embodiment, the chemotactic migration is induced by activation of the epidermal growth factor receptor pathways comprising RAS-MAPK and/or PI3K/AKT signaling pathways in the cell. In still further embodiments, the neoplastic cell in which the migration is inhibited is a cell of a glioma tumor or glioblastoma multiform tumor comprising an EGF receptor mutation, a PTEN mutation, or both an EGF receptor mutation and a PTEN mutation. In a still further embodiment, the EGF receptor mutation is an EGFRvIII mutation. Still further, the invention relates to the use of the dibenzodiazepinone analogues, including Compound 1, for the preparation of a medicament to be administered to a subject in an effective amount to inhibit a migration of a neoplastic or endothelial cell in the subject in need thereof.

An exemplary compound of the present invention is the dibenzodiazepinone analogue of Compound 1. Compound 1 is isolated from strains of actinomycetes, Micromonospora sp. 046-ECO11 and [S01)046. These organisms were deposited on Mar. 7, 2003, and Dec. 23, 2003, respectively, with the International Depositary Authority of Canada (IDAC), Bureau of Microbiology, Health Canada, 1015 Arlington Street, Winnipeg, Manitoba, Canada R3E 3R2, under Accession Nos. IDAC 070303-01 and IDAC 231203-01, respectively.

The methods of the present invention further related to the use of pharmaceutically acceptable salts, esters, solvates and prodrugs of the dibenzodiazepinone analogues and derivatives of the present invention.

One method of obtaining the dibenzodiazepinone analogues of the present invention is by cultivating Micromonospora sp. strain 046-ECO11 or [S01]046 (see, for example U.S. Pat. No. 7,101,872), or a mutant or a variant thereof, under suitable Micromonospora culture conditions, preferably using the fermentation protocol described hereinbelow, to thereby obtain the dibenzodiazepinone analogues. Chemical modification may then be used to produce the derivatives of the dibenzodiazepinone analogues obtained by isolation from the fermentation procedure.

Each of the methods of the present invention further encompasses the use of pharmaceutical compositions and pharmaceutically acceptable formulations comprising a compound of Formula I and its pharmaceutically acceptable salts, esters, solvates and derivatives. Compounds of Formula I are useful as pharmaceuticals, in particular for use as an inhibitor of cancer cell growth, and mammalian lipoxygenase. The pharmaceutical compositions and pharmaceutically acceptable formulations may further comprise a pharmaceutically acceptable carrier.

The following detailed description discloses how to use the compounds of Formula I and compositions containing these compounds to inhibit tumor growth, cell migration and/or specific disease pathways.

Accordingly, certain aspects of the present invention relate to pharmaceutical compositions comprising the dibenzodiazepinone compounds of the present invention together with a pharmaceutically acceptable carrier, and methods of using the pharmaceutical compositions to treat diseases, including cancer, and chronic and acute inflammation, autoimmune diseases, and neurodegenerative diseases.

### I. Definitions

All technical and scientific terms used herein have the same meaning as commonly understood by one of ordinary skill in the art to which this invention belongs. For convenience, the meaning of certain terms and phrases used in the specification, examples, and appended claims, are provided below.

As used herein, the term “farnesyl dibenzodiazepinone” refers to Compound 1, namely 10-farnesyl-4,6,8-trihydroxy-5,10-dihydrodibenzo[b,e][1,4]diazepin-11-one, also referred to as TLN-4601.

As used herein, the terms “dibenzodiazepinone analogue(s)” and equivalent expressions refer to a class of dibenzodiazepinone molecules containing a farnesyl moiety or being derived from a farnesyl moiety, and pharmaceutically acceptable salts, esters, solvates and prodrugs thereof. The term includes each of Compounds 1-100, the compounds of Formula I, and the compounds of Formula II as well as a pharmaceutically acceptable salt, ester, solvate or prodrug of any of these compounds. As used herein, the term “dibenzodiazepinone analogues” includes compounds of this class that can be used as intermediates in chemical syntheses and variants containing different isotopes than the most abundant isotope of an atom (e.g, D replacing H, ¹³C replacing ¹²C, etc). The compounds of the invention are also sometimes referred as “active ingredients”.

As used herein, the “dibenzodiazepinone analogue derivatives”, “chemical derivatives” of dibenzodiazepinone analogues, “derivatives” of dibenzodiazepinone analogues, and equivalent expressions, refer to a class of dibenzodiazepinone molecules produced by chemical modification of the dibenzodiazepinone analogues of the present invention, and to pharmaceutically acceptable salts, esters, solvates and prodrugs thereof. The term includes derivatives produced by chemical modification of each of Compounds 1-100, the compounds of Formula I, and the compounds of Formula II, as well as a pharmaceutically acceptable salt, ester, solvate or prodrug of the derivatives.

As used herein, the term “chemical modification” refers to one or more steps of modifying a dibenzodiazepinone analogue, referred to as “starting material”, by chemical synthesis. Preferred analogues for use as starting materials in a chemical modification process are Compounds 1 to 100, more preferably Compounds 1, 2, 46, 97, 99 and 100. Examples of chemical modification steps include N-alkylations, N-acylations, O-alkylations, O-acylations, aromatic halogenation, and modifications of the double bonds of the farnesyl side chain including, hydrogenation, electrophilic additions (e.g., epoxidation, dihydroxylation, hydration, hydroalkoxylation, hydroamidation, and the like), and double bond cleavage like ozonolysis, and reduction of ozonolysis product. Farnesyl side chain modification reaction can be partial (one or two double bonds modified) or complete (three double bonds modified).

The term “ether” refers to a dibenzodiazepinone analogue derivative obtained by the replacement of a hydrogen atom from an alcohol by an R′ replacement group by an O-alkylation reaction. More particularly, the term ether encompasses ethers of the alcohols in positions 4, 6, and 8.

The term “ester” refers to a dibenzodiazepinone analogue derivative obtained by the replacement of a hydrogen atom from an alcohol by a C(O)R″ replacement group by an O-acylation reaction. The term ester also encompasses ester equivalents including, without limitation, carbonate, carbamate, and the like. More particularly, the term “ester” encompasses esters of the alcohols in positions 4, 6, and 8.

The term “N-alkylated derivative” refers to a dibenzodiazepinone analogue derivative obtained by the replacement of a hydrogen atom of an amine by an R replacement group by an N-alkylation reaction. More particularly, the term “N-alkylated derivative” encompasses derivatives of the amine in position 5.

The term “N-acylated derivative” refers to a dibenzodiazepinone analogue derivative obtained by the replacement of a hydrogen atom of an amine by a C(O)R replacement group by an N-acylation reaction. The term N-acylated derivative further encompasses amide equivalents such as, without limitation, urea, guanidine, and the like. More particularly, the term “N-acylated derivative” encompasses derivatives of the amine in position 5.

The term “receptor” refers to a protein located on the surface or inside a cell that may interact with a different molecule, known as a ligand, to initiate or inhibit a biological response.

As used herein the term “growth factor-driven cancer” refers to any cancer or tumor in which abherent activity of growth factor stimulates autonomus growth associated with the cancer.

As used herein, the term “ligand” refers to a molecule or compound that has the capacity to bind to a receptor and modulate its activity.

As used herein, the terms “binder”, “receptor binder” or “binding agent” refers to a compound of the invention acting as a ligand. The binding agent can act as an agonist, or an antagonist of the receptor. An agonist is a drug which binds to a receptor and activates it, producing a pharmacological response (e.g. contraction, relaxation, secretion, enzyme activation, etc.). An antagonist is a drug which counteracts or blocks the effects of an agonist, or a natural ligand. Antagonism can be competitive and reversible (i.e. it binds reversibly to a region of the receptor in competition with the agonist.) or competitive and irreversible (i.e. antagonist binds covalently to the receptor, and no amount of agonist can overcome the inhibition). Other types of antagonism are non-competitive antagonism where the antagonist binds to an allosteric site on the receptor or an associated ion channel.

As used herein, the term “enzyme inhibitor” or “inhibitor” refers to a chemical that disables an enzyme and inhibits it from performing its normal function.

As used herein, abbreviations have their common meaning. Unless otherwise noted, the abbreviations “Ac”, “Me”, “Et”, “Pr”, “i-Pr”, “Bu”, “Bz” and “Ph”, respectively refer to acetyl, methyl, ethyl, propyl (n- or iso-propyl), iso-propyl, butyl (n-, iso-, sec- or tert-butyl), benzoyl and phenyl. Abbreviations in the specification correspond to units of measure, techniques, properties or compounds as follows: “RT” means retention time, “min” means minutes, “h” means hour(s), “μL” means microliter(s), “mL” means milliliter(s), “mM” means millimolar, “M” means molar, “mmole” means millimole(s), “eq” means molar equivalent(s). “High Pressure Liquid Chromatography” and “High Performance Liquid Chromatography” are abbreviated HPLC.

The term “alkyl” refers to linear, branched or cyclic, saturated hydrocarbon groups. Examples of alkyl groups include, without limitation, methyl, ethyl, n-propyl, isopropyl, n-butyl, pentyl, hexyl, heptyl, cyclopentyl, cyclohexyl, cyclohexylmethyl, and the like. Alkyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, oxo, guanidino and formyl.

The term “C₁₋ₙalkyl”, wherein n is an integer from 2 to 12, refers to an alkyl group having from 1 to the indicated “n” number of carbons. The C₁₋ₙalkyl can be cyclic or a straight or branched chain.

The term “alkenyl” refers to linear, branched or cyclic unsaturated hydrocarbon groups containing, from one to six carbon-carbon double bonds. Examples of alkenyl groups include, without limitation, vinyl, 1-propene-2-yl, 1-butene-4-yl, 2-butene-4-yl, 1-pentene-5-yl and the like. Alkenyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, formyl, oxo and guanidino. The double bond portion(s) of the unsaturated hydrocarbon chain may be either in the cis or trans configuration.

The term “C₂₋ₙalkenyl”, wherein n is an integer from 3 to 12, refers to an alkenyl group having from 2 to the indicated “n” number of carbons. The C₂₋ₙalkenyl can be cyclic or a straight or branched chain.

The term “alkynyl” refers to linear, branched or cyclic unsaturated hydrocarbon groups containing at least one carbon-carbon triple bond. Examples of alkynyl groups include, without limitation, ethynyl, 1-propyne-3-yl, 1-butyne-4-yl, 2-butyne-4-yl, 1-pentyne-5-yl and the like. Alkynyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, formyl, oxo and guanidine.

The term “C₂₋ₙalkynyl”, wherein n is an integer from 3 to 12, refers to an alkynyl group having from 2 to the indicated “n” number of carbons. The C₂₋ₙalkynyl can be cyclic or a straight or branched chain.

The term “cycloalkyl” or “cycloalkyl ring” refers to an alkyl group, as defined above, further comprising a saturated or partially unsaturated carbocyclic ring in a single or fused carbocyclic ring system having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbornyl, and the like. Cycloalkyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl and formyl.

The term “C₃₋ₙcycloalkyl”, wherein n is an integer from 4 to 15, refers to a cycloalkyl ring or ring system or having from 3 to the indicated “n” number of carbons.

The term “heterocycloalkyl”, “heterocyclic” or “heterocycloalkyl ring” refers to a cycloalkyl group, as defined above, further comprising one to four hetero atoms (e.g. N, O, S, P) or hetero groups (e.g. NH, NR^{X}, PO₂, SO, SO₂) in a single or fused heterocyclic ring system having from three to fifteen ring members (e.g. tetrahydrofuranyl has five ring members, including one oxygen atom). Examples of a heterocycloalkyl, heterocyclic or heterocycloalkyl ring include, without limitation, pyrrolidine, tetrahydrofuranyl, tetrahydrodithienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3,1,0]hexanyl, 3-azabicyclo[4,1,0]heptanyl, 3H-indolyl, and quinolizinyl. The foregoing heterocycloalkyl groups, as derived from the compounds listed above may be C-attached or N-attached where such is possible. Heterocycloalkyl, heterocyclic or heterocycloalkyl ring may optionally be substituted with substituents selected from acyl, amino, acylamino, acyloxy, oxo, thiocarbonyl, imino, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl and formyl.

The term “C₃₋ₙheterocycloalkyl”, wherein n is an integer from 4 to 15, refers to a heterocycloalkyl group having from 3 to the indicated “n” number of atoms in the cycle and at least one hetero group as defined above.

The terms “halo” or “halogen” refers to bromine, chlorine, fluorine or iodine substituents.

The term “aryl” or “aryl ring” refers to common aromatic groups having “4n+2” electrons, wherein n is an integer from 1 to 3, in a conjugated monocyclic or polycyclic system and having from five to fourteen ring atoms. Aryl may be directly attached, or connected via a C₁₋₃alkyl group (also referred to as aralkyl). Examples of aryl include, without limitation, phenyl, benzyl, phenethyl, 1-phenylethyl, tolyl, naphthyl, biphenyl, terphenyl, and the like. Aryl groups may optionally be substituted with one or more substituent group selected from acyl, amino, acylamino, acyloxy, azido, alkythio, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl.

The term “C₅₋ₙaryl”, wherein n is an integer from 5 to 14, refers to an aryl group having from 5 to the indicated “n” number of atoms, including carbon, nitrogen, oxygen and sulfur. The C₅₋ₙaryl can be mono or polycyclic.

The term “heteroaryl” or “heteroaryl ring” refers to an aryl ring, as defined above, further containing one to four heteroatoms selected from oxygen, nitrogen, sulphur or phosphorus. Examples of heteroaryl include, without limitation, pyridyl, imidazolyl, pyrimidinyl, pyrazolyl, triazolyl, tetrazolyl, furyl, thienyl, isooxazolyl, thiazolyl, oxazolyl, isothiazolyl, pyrrollyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl groups. Heteroaryl may optionally be substituted with one or more substituent group selected from acyl, amino, acylamino, acyloxy, azido, alkythio, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl. Heteroaryl may be directly attached, or connected via a C₁₋₃alkyl group (also referred to as heteroaralkyl). The foregoing heteroaryl groups, as derived from the compounds listed above, may be C-attached or N-attached where such is possible.

The term “C₅₋ₙheteroaryl”, wherein n is an integer from 5 to 14, refers to an heteroaryl group having from 5 to the indicated “n” number of atoms, including carbon, nitrogen, oxygen and sulphur atoms. The C₅₋ₙheteroaryl can be mono or polycyclic.

The term “amino acid” refers to an organic acid containing an amino group. The term includes both naturally occurring and synthetic amino acids; therefore, the amino group can be but is not required to be, attached to the carbon next to the acid. A C-coupled amino acid substituent is attached to the heteroatom (nitrogen or oxygen) of the parent molecule via its carboxylic acid function. C-coupled amino acid forms an ester with the parent molecule when the heteroatom is oxygen, and an amide when the heteroatom is nitrogen. Examples of amino acids include, without limitation, alanine, valine, leucine, isoleucine, proline, phenylalanine, tryptophan, methionine, glycine, serine, threonine, cysteine, asparagine, glutamine, tyrosine, histidine, lysine, arginine, aspartic acid, glutamic acid, desmosine, ornithine, 2-aminobutyric acid, cyclohexylalanine, dimethylglycine, phenylglycine, norvaline, norleucine, hydroxylysine, allo-hydroxylysine, hydroxyproline, isodesmosine, allo-isoleucine, ethylglycine, beta-alanine, aminoadipic acid, aminobutyric acid, ethyl asparagine, and N-methyl amino acids. Amino acids can be pure L or D isomers or mixtures of L and D isomers.

The compounds of the present invention can possess one or more asymmetric carbon atoms and can exist as optical isomers forming mixtures of racemic or non-racemic compounds. The compounds of the present invention are useful as single isomers or as a mixture of stereochemical isomeric forms. Diastereoisomers, i.e., nonsuperimposable stereochemical isomers, can be separated by conventional means such as chromatography, distillation, crystallization or sublimation. The optical isomers can be obtained by resolution of the racemic mixtures according to conventional processes, including chiral chromatography (e.g. HPLC), immunoassay techniques, or the use of covalently (e.g. Mosher's esters) or non-covalently (e.g. chiral salts) bound chiral reagents to respectively form a diastereomeric ester or salt, which can be further separated by conventional methods, such as chromatography, distillation, crystallization or sublimation. The chiral ester or salt is then cleaved or exchanged by conventional means, to recover the desired isomer(s).

The invention encompasses isolated or purified compounds. An “isolated” or “purified” compound refers to a compound which represents at least 10%, 20%, 50%, 80%, 81%, 82%, 83%, 84%, 85%, 86%, 87%, 88%, 89%, 90%, 91%, 92%, 93%, 94%, 95%, 96%, 97%, 98% or 99% of the mixture by weight, provided that the mixture comprising the compound of the invention has demonstrable (i.e. statistically significant) biological activity including cytostatic, cytotoxic, enzyme inhibitory or receptor binding action when tested in conventional biological assays known to a person skilled in the art.

The term “pharmaceutically acceptable salt” refers to nontoxic salts synthesized from a compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, methanol, ethanol, isopropanol, or acetonitrile are preferred. Another method for the preparation of salts is by the use of ion exchange resins. The term “pharmaceutically acceptable salt” includes both acid addition salts and base addition salts, either of the parent compound or of a prodrug or solvate thereof. The nature of the salt is not critical, provided that it is pharmaceutically acceptable. Exemplary acids used in acid addition salts include, without limitation, hydrochloric, hydrobromic, hydroiodic, nitric, carbonic, sulfuric, sulfonic, phosphoric, formic, acetic, citric, tartaric, succinic, oxalic, malic, glutamic, propionic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactaric, galacturonic acid and the like. Suitable pharmaceutically acceptable base addition salts include, without limitation, metallic salts made from aluminium, calcium, lithium, magnesium, potassium, sodium and zinc or organic salts, such as those made from N,N′-dibenzylethylenediamine, chloroprocaine, choline, diethanolamine, ethylenediamine, N-methylglucamine, lysine, procaine and the like. Additional examples of pharmaceutically acceptable salts are listed in Berge et al (1977) Journal of Pharmaceutical Sciences vol 66, no 1, pp 1-19.

The term “solvate” refers to a physical association of a compound of this invention with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, hemiethanolates, and the like.

The term “pharmaceutically acceptable prodrug” means any pharmaceutically acceptable ester, salt of an ester or any other derivative of a compound of this invention, which upon administration to a recipient, is capable of providing, either directly or indirectly, a compound of this invention or a biologically active metabolite or residue thereof. Particularly favored salts or prodrugs are those with improved properties, such as solubility, efficacy, or bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. As used herein, a prodrug is a drug having one or more functional groups covalently bound to a carrier wherein metabolic or chemical release of the drug occurs in vivo when the drug is administered to a mammalian subject. Pharmaceutically acceptable prodrugs of the compounds of this invention include derivatives of hydroxyl groups such as, without limitation, acyloxymethyl, acyloxyethyl and acylthioethyl ethers, esters, amino acid esters, phosphate esters, sulfonate and sulfate esters, and metal salts, and the like.

### II. Compounds of the Invention

In one aspect, the invention relates to methods of using novel dibenzodiazepinone analogues and derivatives thereof, referred to herein as the compounds of the invention, and to pharmaceutically acceptable salts, esters, solvates and prodrugs thereof.

The compounds of the invention may be characterized as any one of Compounds 1-100 and derivatives thereof produced by the chemical modifications as defined herein. Compounds 2 to 12, 14, 17, 18, 46, 63, 64, 67, 77, 78, 80, 82 to 85, 87, 89, 92, and 95 to 98 may be characterized by any one of their physicochemical and spectral properties, such as mass and NMR.

In another aspect, the invention relates to methods of using dibenzodiazepinone analogues and derivatives thereof, represented by Formula I:

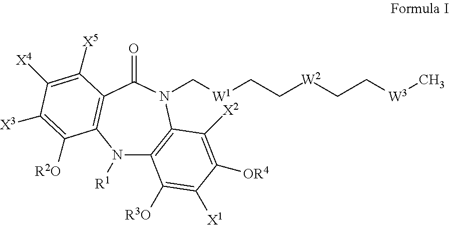

wherein,

W1, W² and W³ are each independently selected from

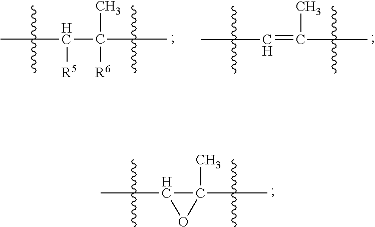

or
the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;

R¹ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;

R², R³, and R⁴ are each independently selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;

R⁵ and R⁶ are each independently selected from the group consisting of H, OH, OC₁₋₆alkyl, NH₂, NHC₁₋₆alkyl, N(C₁₋₆alkyl)₂, and NHC(O)C₁₋₆alkyl;

is R⁷ is selected from the group consisting of H, C₁₋₁₀alkenyl, C₂₋₁₀alkenyl, C₂₋₁₀alkynyl, C₆₋₁₀aryl, C₅₋₁₀heteroaryl, C₃₋₁₀cycloalkyl and C₃₋₁₀heterocycloalkyl;

X¹, X², X³, X⁴, and X⁵ are each H; or one of X¹, X², X³, X⁴ or X⁵ is halogen and the remaining ones are H; and

wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from the group consisting of acyl, amino, acylamino, acyloxy, carboalkoxy, carboxy, carboxyamido, cyano, halo, hydroxyl, nitro, thio, C₅₋₁₀alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl; and an ester, ether, N-alkylated or N-acylated derivative, or a pharmaceutically acceptable salt, solvate or prodrug thereof.

In further aspect, the invention relates to methods of using dibenzodiazepinone analogues and derivatives thereof, represented by Formula II:

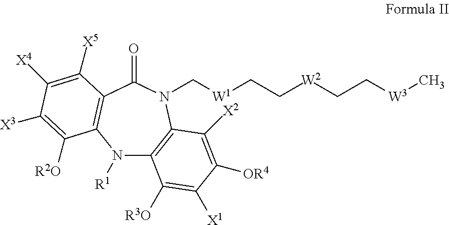

wherein,

Structure of Formula II is as described for structure of Formula I,

with the proviso that when W¹, W² and W³ are all —CH═C(CH₃)—, and R², R³ and R⁴ are all H, then R¹ is not H;

and an ester, ether, N-alkylated or N-acylated derivative, or a pharmaceutically acceptable salt, solvate or prodrug thereof.

In one embodiment, R¹ is H, and all other groups are as previously disclosed. In another embodiment, R¹ is —CH₃, and all other groups are as previously disclosed. In another embodiment, R¹ is C₁₋₁₀alkyl, and all other groups are as previously disclosed. In a subclass of this embodiment, the alkyl group is optionally substituted with a substituent selected from halo, fluoro, C₆₋₁₀aryl, and C₅₋₁₀heteroaryl. In another embodiment, R¹ is —C(O)C₁₋₁₀alkyl, and all other groups are as previously disclosed. In another embodiment, R² is H, and all other groups are as previously disclosed. In another embodiment, R³ is H, and all other groups are as previously disclosed. In another embodiment, R⁴ is H, and all other groups are as previously disclosed. In another embodiment, R², R³ and R⁴ are each H, and all other groups are as previously disclosed. In another embodiment, one of R², R³ and R⁴ is CH₃, the others being each H, and all other groups are as previously disclosed. In another embodiment, two of R², R³ and R⁴ are CH₃, the other being H, and all other groups are as previously disclosed. In another embodiment, R², R³ and R⁴ are each CH₃, and all other groups are as previously disclosed. In another embodiment, R², R³ and R⁴ are each H, and W¹ is —CH═C(CH₃)—, and all other groups are as previously disclosed. In another embodiment, R², R³ and R⁴ are each H, and W² is —CH═C(CH₃)—, and all other groups are as previously disclosed. In another embodiment, R², R³ and R⁴ are each H, and W³ is —CH═C(CH₃)—, and all other groups are as previously disclosed. In another embodiment, R¹ is H and R², R³ and R⁴ are each H, and all other groups are as previously disclosed. In another embodiment, R¹ is H, each of W¹, W², and W³ is —CH═C(CH₃)—, and all other groups are as previously disclosed. In another embodiment, R¹ is H, each of W¹, W², and W³ is —CH₂CH(CH₃)—, and all other groups are as previously disclosed. In another embodiment, X¹ is Br, and each of X², X³, X⁴ and X⁵ are H, and all other groups are as previously disclosed. In another embodiment, if each of W¹, W² and W³ are —CH═C(CH₃)—, and each of R², R³, and R⁴ are H, then R¹ is not H. In further Is embodiment, if each of W¹, W² and W³ are —CH═C(CH₃)—, and each of R², R³, and R⁴ are H, then R¹ is not CH₃. In further embodiment, if each of W¹, W² and W³ are —CH═C(CH₃)—, and each of R², R³, and R⁴ are H, then R¹ is neither H nor CH₃. The invention encompasses all esters, ethers, N-alkylated or N-acylated derivatives, and pharmaceutically acceptable salts, esters, solvates and prodrugs of the foregoing compounds.

The following are exemplary compounds of the invention, such named compounds are not intended to limit the scope of the invention in any way:

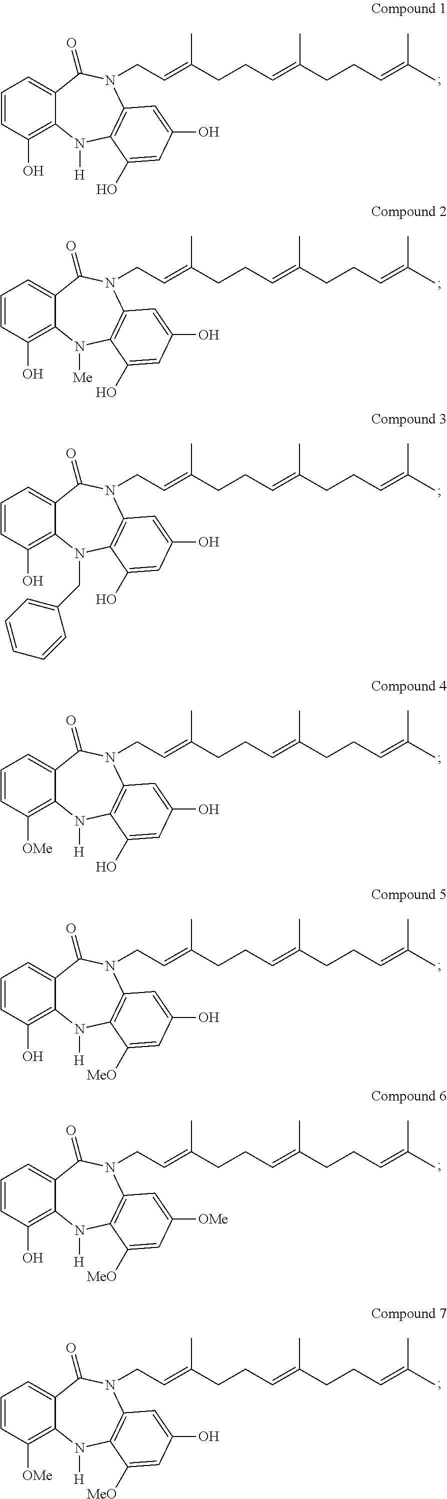

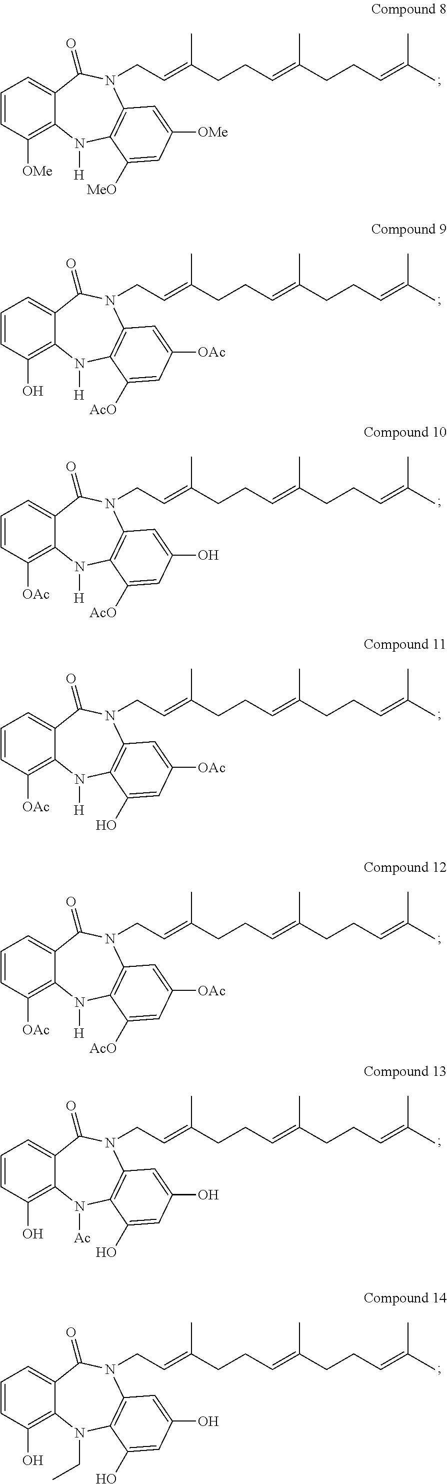

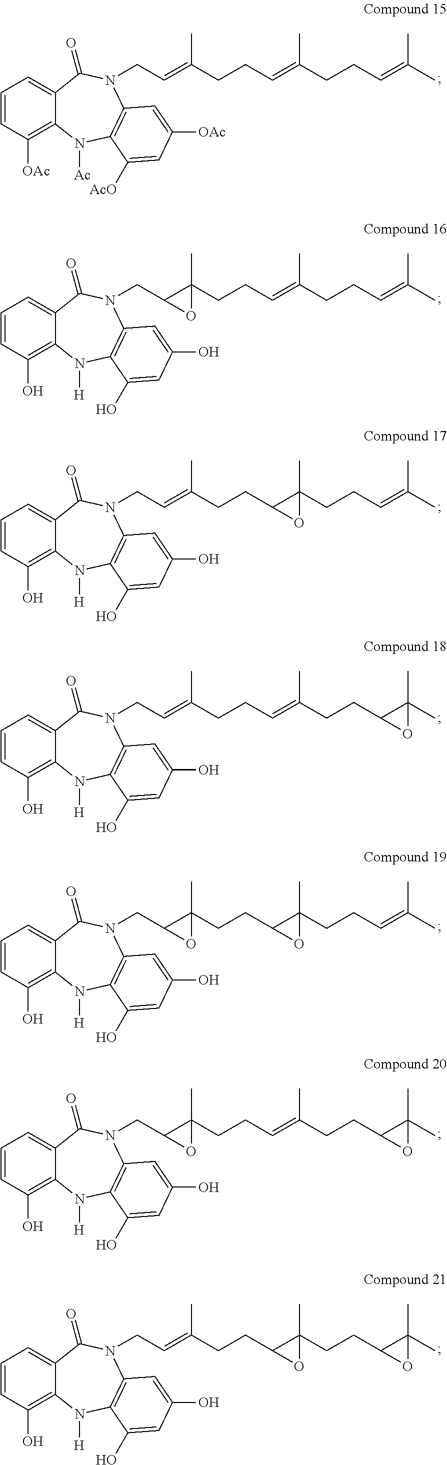

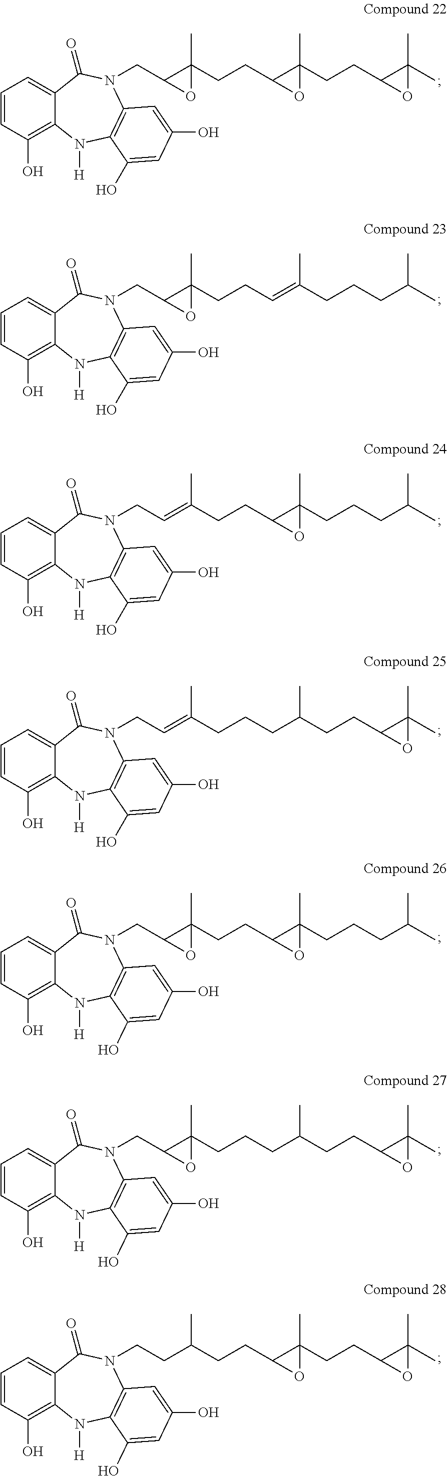

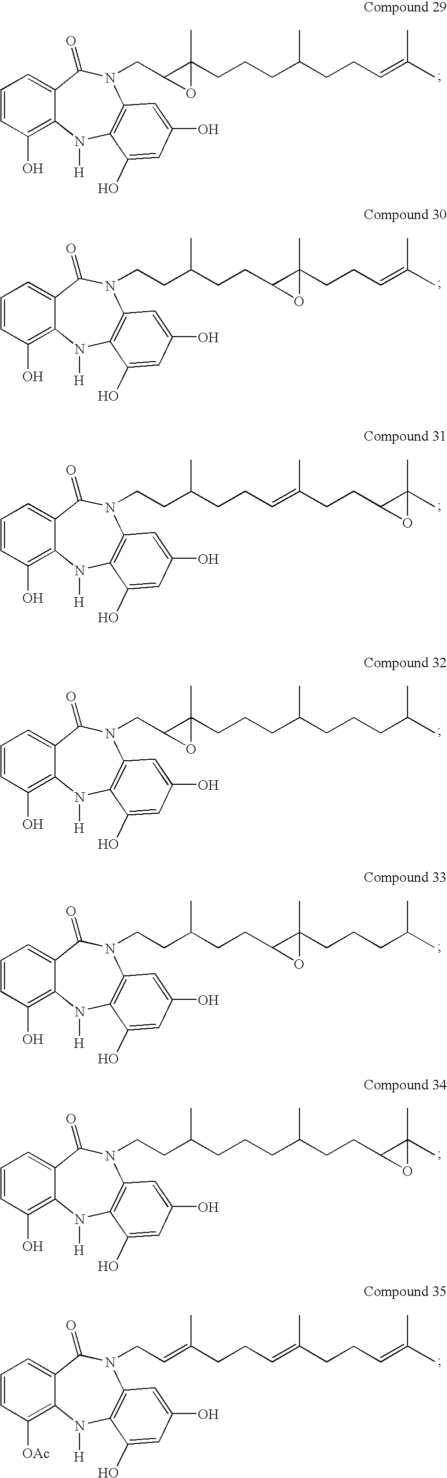

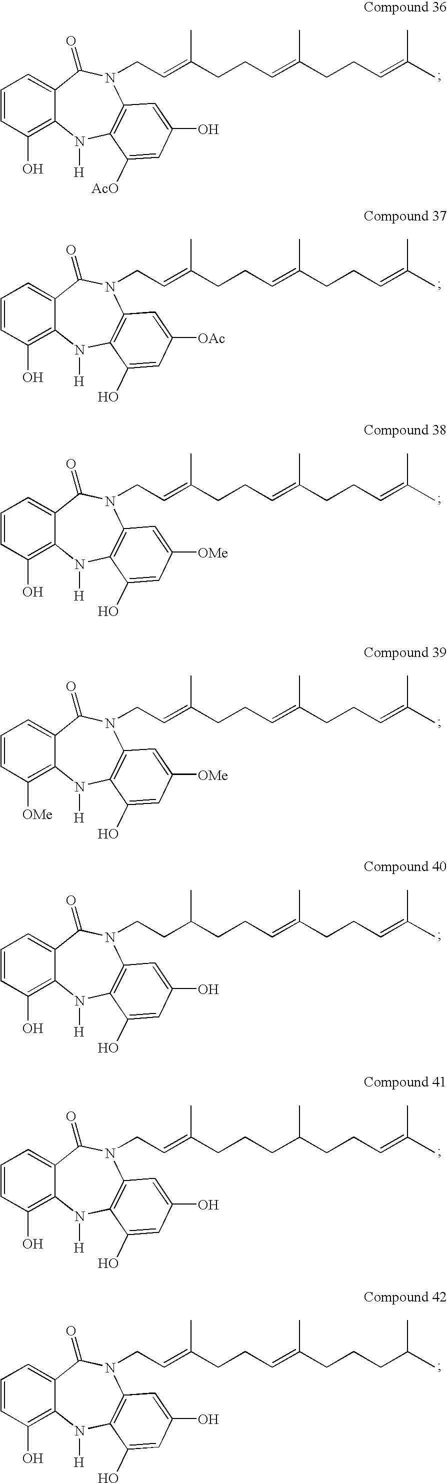

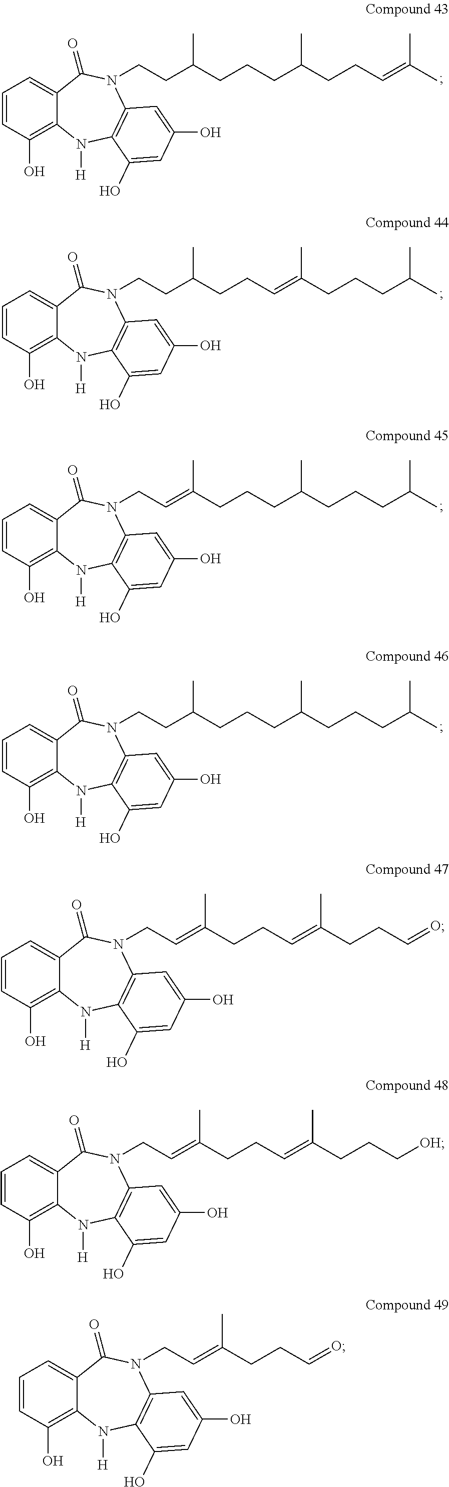

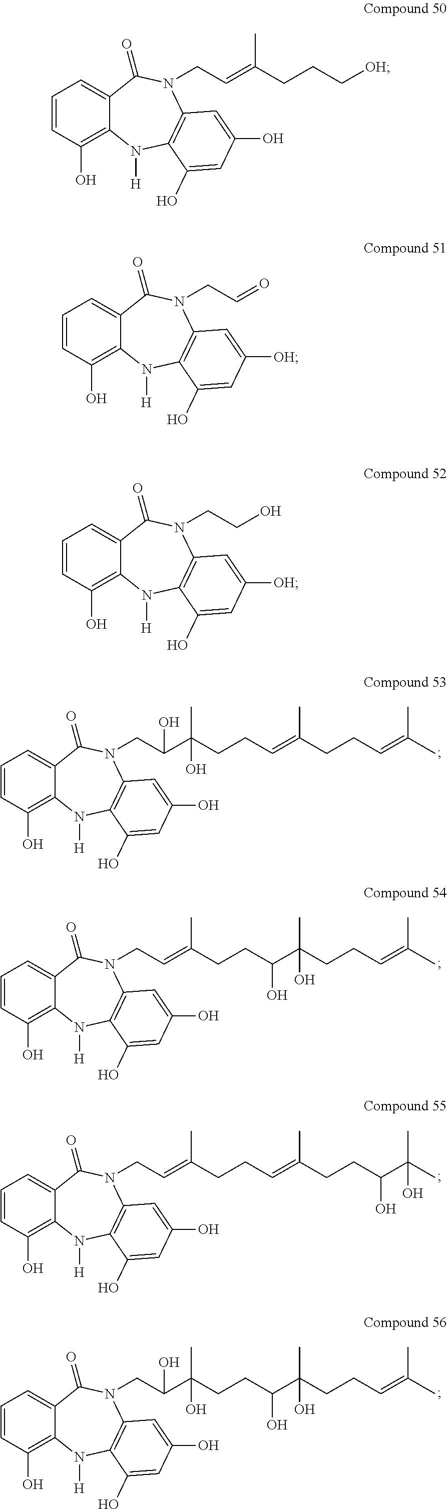

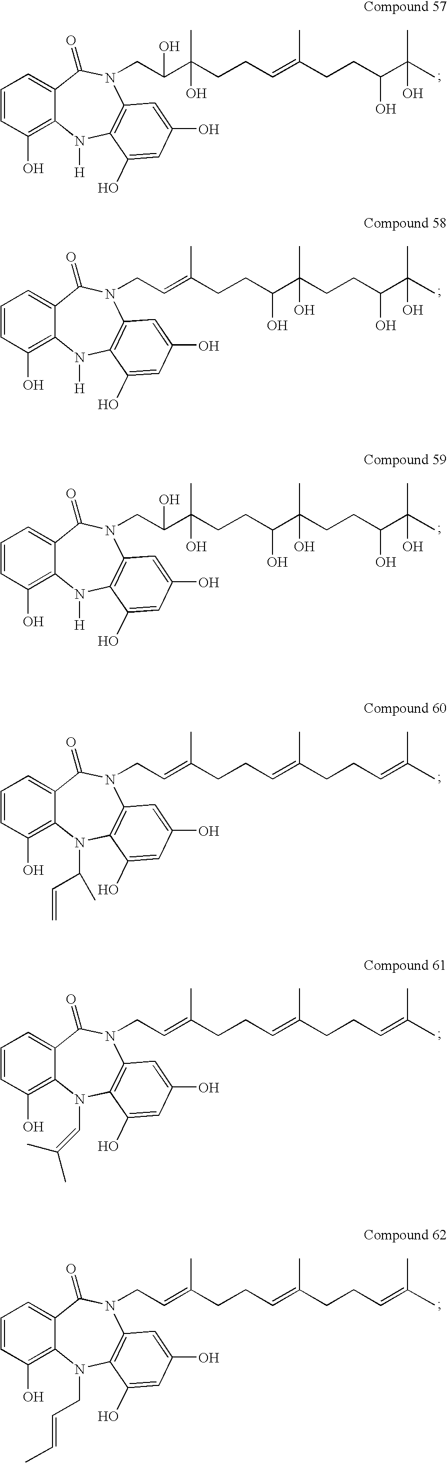

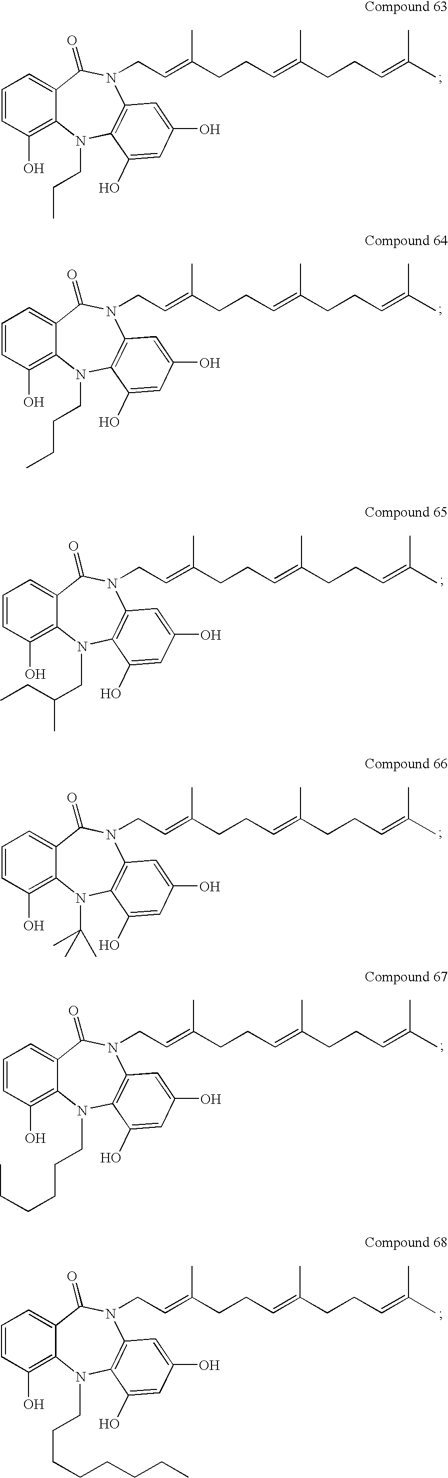

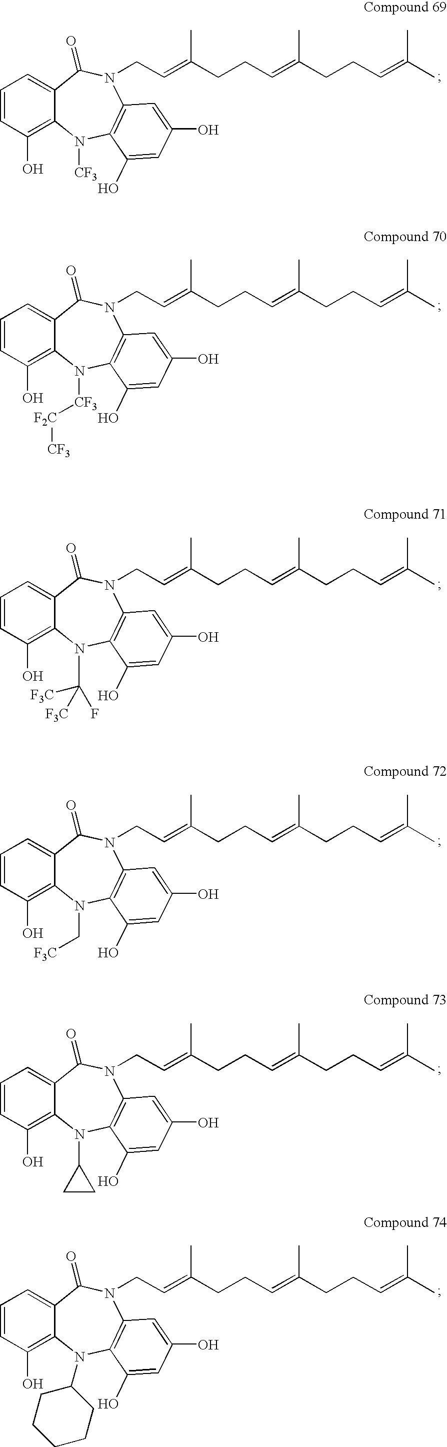

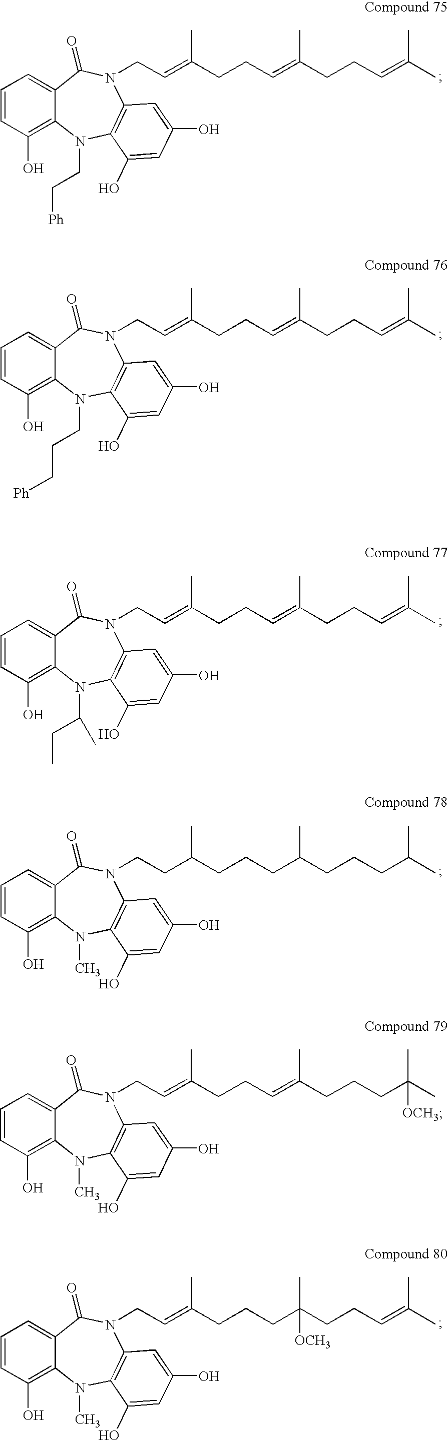

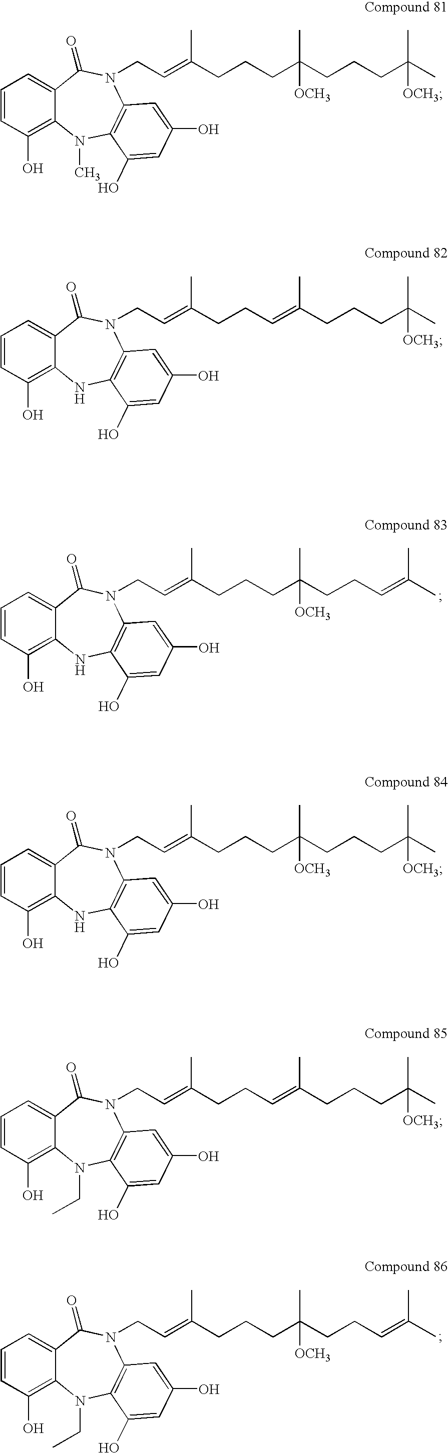

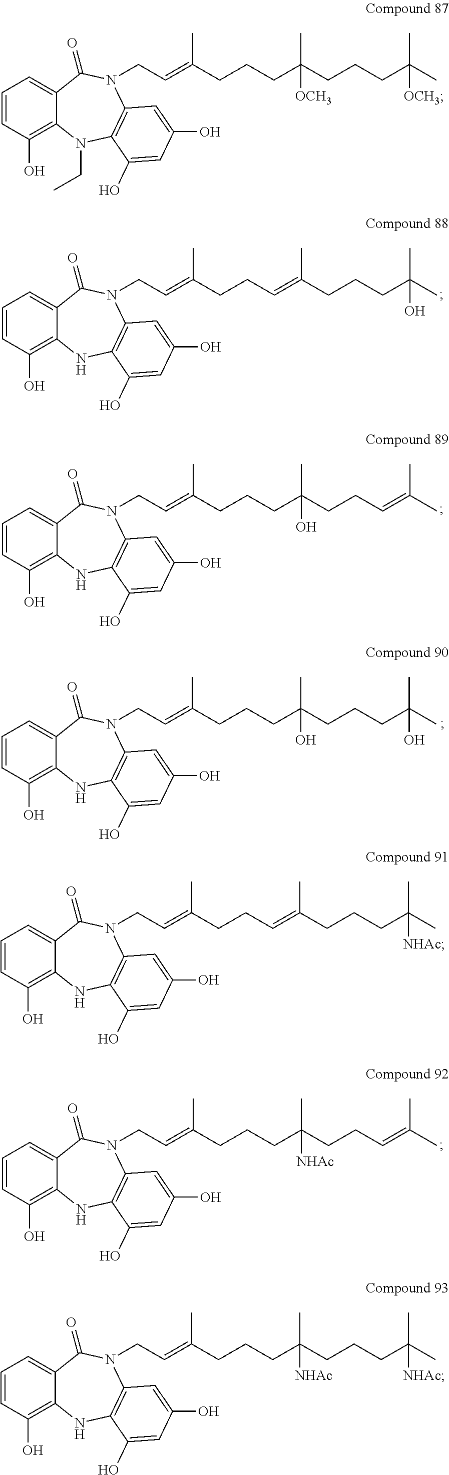

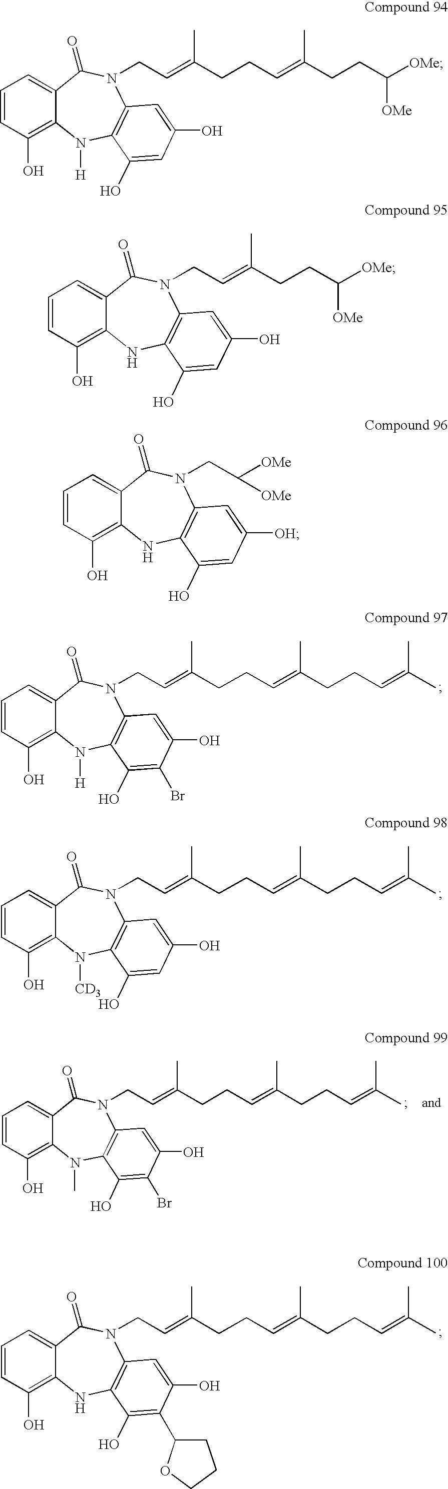

and pharmaceutically acceptable salts, esters, solvates and prodrugs of any one of Compounds 1 to 100.

The invention further provides ethers, esters, N-acylated and N-alkylated derivatives of any of the foregoing Compounds 1-100, as well as pharmaceutically acceptable salts, esters, solvates and prodrugs thereof.

Prodrugs of the compounds of Formula I or II include compounds wherein one or more of the 4, 6 and 8-hydroxy groups, or any other hydroxyl group on the molecule is bounded to any group that, when administered to a mammalian subject, is cleaved to form the free hydroxyl group. Examples of prodrugs include, but are not limited to, acetate, formate, hemisuccinate, benzoate, dimethylaminoacetate and phosphoryloxycarbonyl derivatives of hydroxy functional groups; dimethylglycine esters, aminoalkylbenzyl esters, aminoalkyl esters or carboxyalkyl esters of hydroxy functional groups. Carbamate and carbonate derivatives of the hydroxy groups are also included. Derivatizations of hydroxyl groups also encompassed, are (acyloxy)methyl and (acyloxy)ethyl ethers, wherein the acyl group contains an alkyl group optionally substituted with groups including, but not limited to, ether, amino and carboxylic acid functionalities, or where the acyl group is an amino acid ester. Also included are phosphate and phosphonate esters, sulfate esters, sulfonate esters, which are in alkylated (such as bis-pivaloyloxymethyl (POM) phosphate triester) or in the salt form (such as sodium phosphate ester (—P(O)O—₂Na⁺₂)). For further examples of prodrugs used in anticancer therapy and their metabolism, see Rooseboom et al (2004) Phamacol. Rev vol 56, pp 53-102. When the prodrug contains an acidic or basic moiety, the prodrug may also be prepared as its pharmaceutically acceptable salt.

The compounds of this invention may be formulated into pharmaceutical compositions comprised of a compound of Formula I or II, in combination with a pharmaceutically acceptable carrier, as described in Canadian Patent 2,547,866.

### III. Medical Use in the Treatment of Metastasis, Cell Migration, Neoplasms and for Anti-Angiogenesis

In one aspect, the invention relates to methods for treating a subject having a growth factor-driven cancer. In another aspect, the invention relates to methods for inhibiting growth and/or proliferation and/or migration of a growth factor driven cancer or cancer cells in a subject. As used herein, “subjects” includes animals that can develop growth factor-driven cancers, and includes mammals such as ungulates (e.g. sheeps, goats, cows, horses, pigs), and non-ungulates, including rodents, felines, canines and primates (i.e. human and non-human primates). In a preferred embodiment, the subject is a human.

Angiogenesis is a physiological process involving the formation of new blood vessels from pre-existing vessels. This is a normal process in growth and development, as well as in wound healing. However, this is also a fundamental step in the transition of tumors from a dormant state to a malignant state. Tumor-induced angiogenesis begins with the degradation of the basement membrane. This is accomplished by matrix metalloproteinases (MMPs) secreted by activated endothelial cells which migrate and proliferate, leading to the formation of solid endothelial cell sprouts into the stromal space (Folkman, J, Seminars in Cancer Biology (1992) vol. 3 pp. 65-71; Stetler-Stevenson, WG, Journal of Clinical Investigation (1999) vol. 103 pp. 1237-1241). Angiogenesis is regulated by a series of growth factors and cytokines, such as vascular endothelial growth factor (VEGF), fibroblast growth factor (FGF), and angiogenin. These factors act as both autocrine and paracrine factors that promote angiogenesis. Angiogenesis is also required for the spread of a tumor, or metastasis. Single cancer cells can break away from an established solid tumor, enter the blood vessel, and be carried to a distant site, where they can implant and begin the growth of a secondary tumor. Evidence now suggests that the blood vessel in a given solid tumor may, in fact, be a mosaic of vessels, comprised of endothelial and tumor cells. This mosaicity allows for substantial shedding of tumor cells into the vasculature. The subsequent growth of such metastases will also require a supply of nutrients and oxygen.

Glioblastoma, a type of brain cancer, is part of the larger group of tumors that impact the central nervous system, known as gliomas. Patients with highly recurrent glioblastoma are usually at a more advanced stage of the disease and correspondingly may face altered brain function or death due to the tumor's rapid growth rate. Currently, radiation therapy is the most effective treatment following surgery, and almost all patients receive some form of radiation therapy. Gliomas—tumors of the brain—are among the most angiogenic of all tumors, meaning the tumor has the ability to grow by drawing on blood from surrounding vessels at a very rapid rate. The inhibition of tumor angiogensis may offer the potential as a highly effective form of therapy.

The over-expression of platelet-derived growth factor (PDGF) receptor in low-grade gliomas and epidermal growth factor (EGF) receptor in glioblastoma multiform (GBM) suggest that signaling pathways that are reliant upon these receptors are critical for gliomagenesis. Receptor protein kinases signal through several effector arms, including Ras-MAPK, PI3K/AKT, PLC-γ and JAK-STAT signaling pathways, which regulate cellular proliferation, survival, migration, calcium signaling and cytokine stimulation. In many cancer conditions, growth factor receptors are subject to amplifications and mutation, for example, EGFR is frequently amplified (40-60%) in GBM and is associated with high levels of EGFR mRNA or proteins. In many instances of GBM, the gene is also rearranged during the process of amplification, resulting in several classes of variant EGFR transcripts. The most common rearrangement is a genomic deletion of exons 2-7, resulting in an in-frame deletion of 801 base pairs (bp) of the coding sequence, thus resulting in a generating of a mutant receptor having a truncation of its extracellular domain. This mutant EGFR receptor has been referred to as del2-7 EGFR, AEGFR or EGFRvIII. Studies have shown that the EGFRvIII protein is detected in 60% of GBMs, and the mutant receptor has also been detected in lung, breast and prostate cancer, but not in normal tissues. Both EGFR gene amplification and EGFRvIII expression has been associated with a poor prognosis in patients with GBM.

The best-characterized genetic alterations found in the malignant progression of human gliomas are inactivation of the genes for p53, p16, and retinoblastoma (RB) as well as an amplification of CDK4 and EGFR (reviewed in Maher et al. (2001) Genes and Development, vol. 15: page 1311). However, the most common genetic alteration is loss of heteroxygosity on chromosome 10, which occurs late in tumor development and at a frequency of 70-90% (Fults and Pedone (1993) Genes Chromosomes Cancer, vol. 7, pp 173). The PTEN (for phosphatase and tensin homology) gene was identified as a candidate tumor suppressor gene located at chromosome 10q23.3 and found to be mutated in ˜30% of GBMs (Kato et al. (2000) Clin Cancer Res, vol 6, pp. 3937; Chalhoub and Baker (2009), Annual Review of Pathology, vol. 4, pp. 127-150). The PTEN protein negatively controls the phosphoinositol 3′-kinase/AKT pathway; in the absence of PTEN, AKT activity is elevated leading to increased proliferation and inhibition of apoptosis (Holland et al., (2000), Nature Genetics, vol. 25 pp. 55). AKT is activated in 70% of gliomas (Hans-Kogan et al (1998) Curr Biol vol.8 pp. 1195-1198).

In non-neoplastic diseases, for example in neovascular (wet) age-related macular degeneration, angiogenesis can also play a role in the development and maintenance of the disease state. As noted in Ng and Adamis (Ng, EWM and Adamis, AP, Canadian Journal of Ophthalmology (2005) vol. 40, pp. 352-368), the underlying cause of the vision loss in this malady is considered to be as a result of choroidal neovascularization. Symptomatic of the disease, such angiogenesis results in a growth of capillaries into the retina, eventually resulting in an occlusion of the vision of an afflicted individual. As further reviewed by Ng and Adamis (2005), the choroidal neovascularization process is thought to be initiated in response to metabolic distress (stemming, for example, from an accumulation of lipid metabolic byproduct, a reduction in choriocapillaris blood flow, oxidative stress and alterations in Bruch's membrane), whereby retinal pigment epithelium cells and the retina produce factors, such as VEGF, that result in choroidal neovascularization. Accordingly, agents that may reduce or inhibit the initiation and/or continuation of the neovascularization process would be beneficial in the treatment of AMD.

As used herein, the terms “neoplasm”, “neoplastic disorder”, “neoplasia” “cancer,” “tumor” and “proliferative disorder” refer to abnormal state or condition characterized by rapidly proliferating cell growth which generally forms a distinct mass that show partial or total lack of structural organization and functional coordination with normal tissue. A “neoplastic cell” is a cell of such a mass, i.e., a cell of a neoplasm or tumor. The terms are meant to encompass hematopoietic neoplasms (e.g. lymphomas or leukemias) as well as solid neoplasms (e.g. sarcomas or carcinomas), including all types of pre-cancerous and cancerous growths, or oncogenic processes, metastatic tissues or malignantly transformed cells, tissues, or organs, irrespective of histopathologic type or stage of invasiveness. Hematopoietic neoplasms are malignant tumors affecting hematopoietic structures (structures pertaining to the formation of blood cells) and components of the immune system, including leukemias (related to leukocytes (white blood cells) and their precursors in the blood and bone marrow) arising from myeloid, lymphoid or erythroid lineages, and lymphomas (relates to lymphocytes). Solid neoplasms include sarcomas, which are malignant neoplasms that originate from connective tissues such as muscle, cartilage, blood vessels, fibrous tissue, fat or bone. Solid neoplasms also include carcinomas, which are malignant neoplasms arising from epithelial structures (including external epithelia (e.g., skin and linings of the gastrointestinal tract, lungs, and cervix), and internal epithelia that line various glands (e.g., breast, pancreas, thyroid). Examples of neoplasms that are particularly susceptible to treatment by the methods of the invention include leukemia, and hepatocellular cancers, sarcoma, vascular endothelial cancers, breast cancers, central nervous system cancers (e.g. astrocytoma, gliosarcoma, neuroblastoma, oligodendroglioma and glioblastoma), prostate cancers, lung and bronchus cancers, larynx cancers, esophagus cancers, colon cancers, colorectal cancers, gastro-intestinal cancers, melanomas, ovarian and endometrial cancer, renal and bladder cancer, liver cancer, endocrine cancer (e.g. thyroid), and pancreatic cancer.

In the methods of the present invention, the dibenzodiazepinone analogue or derivative is brought into contact with or introduced into a cancerous cell or tissue, or an endothelial cell. In general, the methods of the invention for delivering the compositions of the invention in vivo utilize art-recognized protocols for delivering therapeutic agents to a subject with the only substantial procedural modification being the substitution of the compound of the present invention for the therapeutic agent in the art-recognized protocols. The route by which the compound is administered, as well as the formulation, carrier or vehicle will depend on the location as well as the type of the neoplasm. A wide variety of administration routes can be employed. The compound may be administered by intravenous or intraperitoneal infusion or injection. For example, for a solid neoplasm that is accessible, the compound of the invention may be administered by injection directly into the neoplasm. For a hematopoietic neoplasm the compound may be administered intravenously or intravascularly. For neoplasms that are not easily accessible within the body, such as metastases or brain tumors, the compound may be administered in a manner such that it can be transported systemically through the body of the mammal and thereby reach the neoplasm and distant metastases for example intrathecally, intravenously or intramuscularly or orally. Alternatively, the compound can be administered directly to the tumor. The compound can also be administered subcutaneously, intraperitoneally, topically (for example for melanoma), rectally (for example colorectal neoplasm) vaginally (for example for cervical or vaginal neoplasm), nasally or by inhalation spray (for example for lung neoplasm).

For use in the methods of inhibiting cellular migration of the present invention, the dibenzodiazepinone analogue or derivative is administered in an amount that is sufficient to inhibit the migration of a cell, whether in vitro or in vivo. The terms “inhibit” and “inhibition”, with regard to the migration of a cell, refers to a decrease in the migratory activity of a cell, whether it be a neoplastic cell, an endothelial cell, or some other cell type. An “effective amount” a compound of the present inventive is one that results in such inhibition when administered to a subject, or when brought into contact with a neoplastic cell or endothelial cell. The inhibiton can be an inhibition of about 10%, 20%, 30%, 40%, 50%, 60%, 70%, 80%, 90%, 95%, 96%, 97%, 98%, 99%, or 100% when compared to a neoplastic cell or endothelial cell not treated with a compound of the present invention. The inhibition of cellular migration according to each method of the invention can be monitored in several ways. Cells grown in vitro can be treated with the compound and monitored for migration relative to the same cells cultured in the absence of the compound. A cessation of migration or a slowing of the migration rate, e.g., by 50% or more is indicative of inhibition of cell migration. Alternatively, migration can be monitored by administering the compound to an animal model. Examples of experimental non-human animal models are known in the art and described below and in the examples herein. A cessation of migration in animals treated with the compound relative to control animals not treated with the compound is indicative of significant inhibition of cellular migration.

As used herein an “inhibitory amount” of a compound of the present invention also refers to an amount of a dibenzodiazepinone analogue or derivative of the present invention that is sufficient to inhibit migration. Such inhibition may be an inhibition of about 10%, 20%, 30%, 40%, 50%, 60%, 70%, 80%, 90%, 95%, 96%, 97%, 98%, 99%, or 100% relative to a cell or tumor that is not contacted with a compound of the present invention.

The term “inhibiting migration of a cell” refers to an inhibition that may be an inhibition of about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the migration activity of a cell contacted with a compound of Formula I when compared to a migration activity of a like cell that has not been contacted with a compound of Formula I.

### Examples

Unless otherwise noted, all reagents were purchased from Sigma-Aldrich (St. Louis, Mo.).

Unless otherwise indicated, all numbers expressing quantities of ingredients, properties such as molecular weight, reaction conditions, molar equivalents (eq), percentage of binding and/or inhibition, GI₅₀, IC₅₀ and so forth used in the specification and claims are to be understood as being modified in all instances by the term “about”. Accordingly, unless indicated to the contrary, the numerical parameters set forth in the present specification and attached claims are approximations. At the very least, and not as an attempt to limit the application of the doctrine of equivalents to the scope of the claims, each numerical parameter should at least be construed in light of the number of significant figures and by applying ordinary rounding techniques. Notwithstanding that the numerical ranges and parameters setting forth the broad scope of the invention are approximations, the numerical values set in the examples, Tables and Figures are reported as precisely as possible. Any numerical values may inherently contain certain errors resulting from variations in experiments, testing measurements, statistical analyses and such.

Although methods and materials similar or equivalent to those described herein can be used in the practice or testing of the present invention, suitable methods and materials are described below. In case of conflict, the present specification, including definitions, will control.

### Example 1

### Pharmacological Activity Profile

Compound 1 and Compounds 2 to 12 and Compound 46 were tested for binding against a variety of enzymes and/or receptors. The enzymes or receptors used in these assays were known to be involved in anticancer activity of known compounds, as well as other diseases, or related to such enzymes or receptors.

### A. Enzymes and Receptors:

5-Lipoxygenase (5-LO) catalyzes the oxidative metabolism of arachidonic acid to 5-hydroxyeicosatetraenoic acid (5-HETE), the initial reaction leading to formation of leukotrienes. Eicosanoids derived from arachidonic acid by the action of lipoxygenases or cycloxygenases have been found to be involved in acute and chronic inflammatory diseases (i.e. asthma, multiple sclerosis, rheumatoid arthritis, ischemia, edema) as well in neurodegeneration (Alzheimer's disease), aging and various steps of carcinogenesis, including tumor promotion, progression and metastasis. The aim of this study was to determine whether Compound 1, is able to block the formation of leukotrienes by inhibiting the enzymatic activity of human 5-LO.

Acyl CoA-Cholesterol Acyltransferase (ACAT) converts cholesterol to cholesteryl esters and is involved in the development of artherioscerosis.

Cyclooxygenase-2 (COX-2) enzyme is made only in response to injury or infection. It produces prostaglandins involved in inflammation and the immune response. Elevated levels of COX-2 in the body have been linked to cancer.

The peripheral benzodiazepine receptor (PBR or PBenzR) is a well-characterized receptor known to be directly involved in diseases states. PBR is involved in the regulation of immune responses. These diseases states include inflammatory diseases (such as rheumatoid arthritis and lupus), parasitic infections and neurodegenerative diseases (such as Alzheimer's, Huntington's and Multiple Sclerosis). This receptor is known to be involved in anticancer activity of known compounds.

Leukotriene, Cysteinyl (CysLT₁) is involved in inflammation and CysLT₁-selective antagonists are used as treatment for bronchial asthma. CysLT₁ and 5-LO were found to be upregulated in colon cancer.

GABA_{A}, the Central Benzodiazepine Receptor (CBenzR or CBR) is involved in anxiolitic activities.

### B. General Procedures:

The procedures used were based on known assays: ACAT (from rat; Ref: Largis et al (1989), J. Lipid. Res., vol 30, 681-689), COX-2 (human; Ref: Riendeau et al (1997), Can. J. Physiol. Pharmacol., vol 75, 1088-1095 and Warner et al (1999), Pro. Natl. Acad Sci. USA, vol 96, 7563-7568), 5-LO (human; Ref: Carter et al (1991), J. Pharmacol. Exp. Ther., vol 256, no 3, 929-937, and Safayhi et al (2000), Planta Medica, vol 66, 110-113), PBR (from rat; Le Fur et al (1983), Life Sci. USA, vol 33, 449-457), CysLT₁ (human; Martin et al (2001), Biochem. Pharmacol., vol 62, no 9, 1193-1200) and CBR (from rat; Damm et al (1978), Res. Comm. Chem. Pathol. Pharmacol., vol 22, 597-600 and Speth et al (1979), Life Sci., vol 24, 351-357).

### C. Binding Assay of Compound 1 on 5-LO:

Human peripheral blood mononuclear cells (PMNs) were isolated through a Ficoll-Paque density gradient. PMNs were stimulated by addition A23187 (30 μM final concentration). Stimulated PMNs were adjusted to a density of 5×10⁶ cells/mL in HBBS medium and incubated with the vehicle control (DMSO), Compound 1 (at final concentrations of 0.1, 0.5, 1, 2.5, 5 and 10 μM) and NDGA as positive control (at final concentrations of 3, 1, 0.3, 0.1 and 0.03 μM) for 15 minutes at 37° C. Following incubation, samples were neutralized with NaOH and centrifuged. Leukotriene B4 content was measured in the supernatant using an Enzyme Immunosorbant Assay (EIA) assay. The experiment was performed in triplicate.

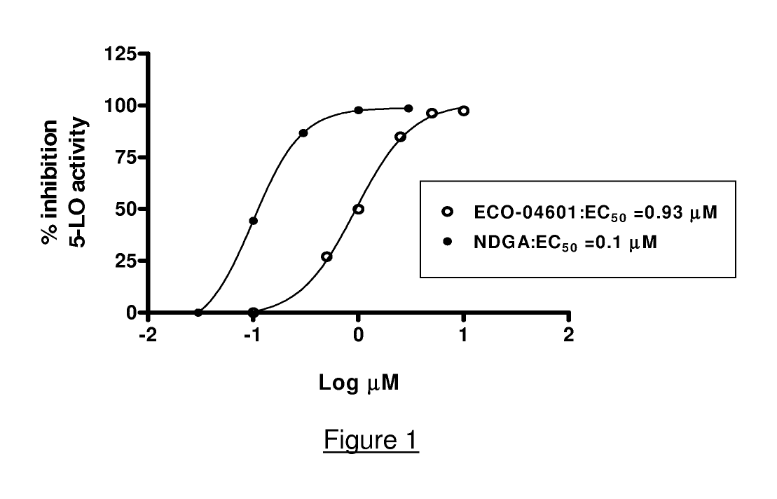

Results shown in FIG. 1 demonstrated that Compound 1 inhibited the activity of human 5-LO with an apparent IC₅₀=0.93 μM (versus 0.1 μM for the positive control NDGA) and therefore displays anti-inflammatory properties.

### D. Percentage Inhibition or Binding of Compounds 1-12 and 46:

Binding assays were done for each of Compounds 1-12 and 46 using ACAT, COX-2, 5-LO, PBR and CysLT₁ enzymes. The procedures used are based on the respective references mentioned above and the conditions are summarized in Tables 1 (enzyme assays) and 2 (radioligand receptor assays).

TABLE 1
Enzyme Assays Conditions

| | Source | Substrate | Pre-I ^{a} | I ^{b} |
|---|---|---|---|---|
| ACAT ^{c} | Wistar rat hepatic | 12.7 μM [¹⁴C]palmitoyl CoA | 15 min/37° C. | 10 min/37° C. |
| | microsomes | | | |
| COX-2 ^{d} | Human recombinant | 0.3 μM arachidonic acid | 15 min/37° C. | 5 min/37° C. |
| | insect Sf21 cells | | | |
| 5-LO ^{e} | Human PBML cells | Arachidonic acid | 15 min/37° C. | 15 min/37° C. |

^{a} Pre-Incubation Time/Temperature
^{b} Incubation Time/Temperature
^{c} Incubation buffer: 0.2 M phosphate buffer (pH 7.4 at 25° C.); Method: Quantitation of
[¹⁴C]cholesterol ester by column chromatography.
^{d} Incubation buffer: 100 mM Tris-HCl, pH 7.7, 1 mM glutathione, 1 μM hematin, 500 μM phenol;
Method: EIA quantitation of PGE₂.
^{e} Incubation buffer: HBSS (Hank's balanced salt solution); Method: EIA quantitation of LTB₄.

TABLE 2
Radioligand Binding Assays Conditions ^{a}

| | | | | Non-spec |
|---|---|---|---|---|
| | Source | Ligand | I ^{b} | ligand |
| PBR ^{c} | Wistar rat heart | 0.3 nM [³H]PK-11195 | 15 min/25° C. | Dipyridamole ^{f} |
| CysLT₁ ^{d} | Human | 0.3 nM [³H]leukotriene | 30 min/25° C. | Leukotriene D₄ ^{g} |
| | recombinant CHO- | D₄ | | |
| | K1 cells | | | |
| CBR ^{e} | Wistar rat brain | 1 nM [³H]flunitrazepam | 60 min/25° C. | Diazepam ^{h} |

a. Quantitation Method: Radioligand binding
b. Incubation Time/Temperature
c. Incubation buffer: 50 mM Tris-HCl, pH 7.5, 10 mM MgCl₂ at 25° C.
d. Incubation buffer: 50 mM Tris-HCl, pH 7.4, 5 mM CaCl₂, 5 mM MgCl₂, 100 μg/mL bacitracin,
1 mM benzamidine, 0.1 mM PMSF.
e. Incubation buffer: 50 mM Na-K phosphate, pH 7.4 at 25° C.
f. Non specific ligand: 100 μM, K_{D}: 2.3 nM, Bₘₐₓ: 0.17 pmol/mg protein, Specific binding: 90%
g. Non specific ligand: 0.3 μM, K_{D}: 0.21 nM, Bₘₐₓ: 3 pmol/mg protein, Specific binding: 93%
h. Non specific ligand: 10 μM, K_{D}: 4.4 nM, Bₘₐₓ: 1.2 pmol/mg protein, Specific binding: 91%

Binding Assays were done at constant concentration of the compound, in 1% DMSO as vehicle, and are specified below each enzyme/receptor type in Table 3. Significance was obtained when a result was ≧50% binding or inhibition (underlined).

TABLE 3
Percentage of inhibition or binding activity

| | ACAT | COX-2 | 5-LO | PBR | CysLT₁ | CBR |
|---|---|---|---|---|---|---|
| Compound | (10 μM) | (4 μM) | (4 μM) | (1 μM) | (4 μM) | (10 μM) |
| 1 | 90 | 96 | 99 | 80 | 92 | 39 |
| 2 | 51 | 92 | 93 | 65 | 75 | 22 |
| 3 | 63 | 76 | 72 | 11 | 59 | 10 |
| 4 | 65 | 78 | 98 | 92 | 64 | 12 |
| 5 | 60 | 63 | 98 | 68 | 72 | 21 |
| 6 | 54 | 45 | 71 | 75 | 24 | 14 |
| 7 | 95 | 26 | 63 | 65 | 15 | 21 |
| 8 | 40 | 19 | −13 | 55 | 13 | 1 |
| 9 | 77 | 44 | 96 | 32 | 70 | 10 |
| 10 | 90 | 45 | 97 | 86 | 67 | 5 |
| 11 | 71 | 57 | 97 | 39 | 74 | 20 |
| 12 | 83 | 30 | 86 | 39 | 33 | −24 |
| 46 | 8 | 95 | 65 | −1 | 71 | 27 |

All of the exemplified Compounds 1-12 and 46 possessed inhibition and/or binding activity. None of them significantly bound the central benzodiazepine receptor (CBR), which demonstrated that selectivity for the peripheral receptor was present.

PBR binding studies using multiple dilutions indicated that Compound 1 had an inhibition concentration (IC₅₀) value of 0.291 μM and an inhibition constant (Ki) of 0.257 μM, compared to the binding results above, which showed an IC50 above 10 μM in the inhibition of CBR.

### Example 2

### In Vitro Profiling of the Compounds of the Invention

In vitro cytotoxic activities of exemplified Compounds are shown in Table 4, along with hemolytic activity of each compound. Compounds were tested in four human tumor cell lines: HT-29 (colorectal carcinoma), SF268 (CNS), MDA-MB-231 (mammary gland adenocarcinoma) and PC-3 (prostate adenocarcinoma).

Procedures are described below.

TABLE 4
In vitro Cytotoxic Activities and Hemolysis

| Com- | | | | MDA-MB- | |
|---|---|---|---|---|---|
| pound | HT-29 | SF-268 | PC-3 | 231 | Hemolysis ^{a} |
| No: | (GI₅₀ μM) | (GI₅₀ μM) | (GI₅₀ μM) | (GI₅₀ μM) | (ED₅₀ μg/mL) |
| 1 | 11.2/9.33 | 1.96/1.55 | 1.95/3.76 | 1.79/3.18 | 7.6 |
| 2 | 0.65 | 0.12 | 0.45 | 0.24 | 5.12 |
| 3 | 7.3 | 5.73 | 5.36 | 6.32 | >64 |
| 4 | 14.7 | 4.97 | 5.86 | 11.3 | >64 |
| 5 | 14.4 | 13.4 | 15.6 | 20.5 | >64 |
| 6 | >30 | 18.9 | 19.0 | 24.6 | >64 |
| 7 | 14.1 | 18.5 | 14.6 | 17.4 | >64 |
| 9 | 12.6 | 1.88 | 1.44 | 2.48 | >64 |
| 10 | 13.0 | 2.02 | 1.35 | 1.55 | >64 |
| 11 | 16.0 | 5.79 | 5.35 | 7.72 | 9.8 |
| 12 | 9.33 | 1.95 | 1.2 | 2.79 | >64 |
| 14 | 2.04 | 0.76 | 1.15 | 2.16 | 43.9 |
| 17 | >30 | 13.4 | 18.7 | >30 | 35.0 |
| 18 | >30 | 7.45 | >30 | >30 | >64 |
| 46 | 4.26 | 0.72 | 0.90 | 0.59 | 13.9 |
| 63 | 2.57 | 0.89 | 1.25 | 2.27 | >64 |
| 64 | 2.5 | 0.56 | 1.14 | 1.39 | >64 |
| 67 | 2.44 | 0.53 | 1.33 | 1.92 | >64 |
| 77 | 13.9 | 3.31 | 17.1 | 5.62 | 60.9 |
| 78 | 0.29 | 0.07 | 0.23 | 0.24 | 9.89 |
| 80 | 1.43 | 0.33 | 1.80 | 1.02 | >64 |
| 82 | 23.6 | 4.75 | 13.4 | 11.0 | >64 |
| 83 | 19.6 | 9.74 | 13.2 | 6.71 | 12.4 |
| 84 | 21.5 | 3.49 | 16.4 | 23.5 | >32 |
| 85 | 1.89 | 1.73 | 1.08 | 2.19 | >64 |
| 87 | 1.83 | 0.91 | 1.39 | 2.40 | >64 |
| 89 | >30 | 13.7 | 13.5 | 25.3 | >64 |
| 92 | >30 | 13.5 | 16.6 | 11.1 | >64 |
| 97 | 2.02 | 2.04 | 1.19 | 2.02 | 15.1 |
| 98 | 0.69 | 0.16 | 0.82 | 0.51 | 4.5 |

^{a} Hemolysis is measured as the concentration necessary to achieve 50% hemolysis of SRBC (Amphotericin B:4 μg/mL)

In vitro cytotoxic activities of Compounds in Table 4 were determined using propidium iodide (PI). Briefly, two 96-well plates were seeded in duplicate with each cell line at the appropriate inoculation density (HT29: 3,000; SF268: 3,000; PC-3: 3,000; and MDA-MB-231: 7,500 cells) and according to the technical data sheet of each cell line (rows A-G, 75 μL of media per well). Row H was filled with medium only (150 μL, negative control-medium). The plates were incubated at appropriate temperature and CO₂ concentration for 24 hrs.

Test Compounds were prepared as 15× stock solutions in appropriate medium and corresponding to 450, 45, 0.45, 0.045, and 0.0045 μM (prepared the day of the experiment). An aliquot of each was diluted 7.5-fold in appropriate test medium to give a set of six 2× concentration solutions (60, 6, 0.6, 0.06, 0.006, and 0.0006 μM). A 75 μL aliquot of each concentration was added to each corresponding well (rows A to F) of the second plate. Row G was filled with 75 μL of medium/0.6% DMSO (negative control-cells). The second plate was incubated at appropriate temperature and CO₂ concentration for 96 hrs.

First Plate: PI (30 μL, 50 μg/mL) was added to each well of the first plate without removing the culture medium. The plate was centrifuged (Sorvall Legend-RT, swinging bucket) at 3500 rpm/10 min. Fluorescence intensity (Thermo, Varioskan, λₑₓ: 530 nm; λₑₘ: 620 nm) was measured to give the first measurement, dead cells (DC at T₀; before freezing). Two round of Freeze (−80° C)/Thaw (37° C.) were done. Fluorescence intensity was determined to give the second measure, total cells (TC at T₀; after freeze/thaw)

Second plate was processed as the first one, except there were three rounds of freeze/thaw instead of two. First measurement gave the treated dead cells value (TDC), and the second measurement gave the treated total cells value (TTC). Both values were collected for each treated well and control (CTC and CDC).

Each value (DC, TC, TDC, TTC, CTC and CDC) was corrected by removing the background value (medium only) to give the value (FU_{DC(T=0)}, FU_{TC(T=0)}, FU_{TDC}, FU_{TTC}, FU_{CTC} and FU_{CDC}) used in the calculation of the T/C (%) (Treated/Control) for each concentration. T/C (%) for each concentration is calculated using the following formula:

$$T \text{/} C ( \% ) = \frac{( {FU}_{TTC} - {FU}_{TDC} ) - ( {FU}_{TC ( T = 0 )} - {FU}_{DC ( T = 0 )} ) \times 100}{( {FU}_{CTC} - {FU}_{CDC} ) - ( {FU}_{TC ( T = 0 )} - {FU}_{DC ( T = 0 )} )}$$

The GI₅₀ value emphasizes the correction for the cell count at time zero for cell survival. The T/C values are transposed in a graph to determine GI₅₀ values, the concentration at with the T/C is 50%.

### Example 3

### Pharmacokinetic Profiles

Compounds 1 and 2 were separately dissolved in ethanol (5%), Polysorbate 80 (15%), PEG 400 (5%) and dextrose (5%) at a final concentration of 6 mg/ml. Prior to dosing, animals (female Crl: CD1 mice; 6 weeks of age, 22-24 g) were weighed, randomly selected and assigned to the different treatment groups. Compound 1 and Compound 2 were administered by the intravenous (IV) or intraperitoneal (IP) route to the assigned animals. The dosing volume of Compounds 1 and 2 was 5 mL per kg body weight. Animals were anesthetized with 5% isoflurane prior to bleeding. Blood was collected into microtainer tubes containing the anticoagulant K₂EDTA by cardiac puncture from each of 4 animals per bleeding timepoint (2 min, 5 min, 15 min, 30 min, 1 h, 2 h, 4 h and 8 h). Following collection, the samples were centrifuged and the plasma obtained from each sample was recovered and stored frozen (at approximately −80° C.) pending analysis. Samples were analysed by LC/MS/MS. Standard curve ranged from 25 to 2000 ng/mL with limit of quantitation (LOQ)≦25 ng/mL and limit of detection (LOD) of 10 ng/mL.

Plasma values of Compounds 1 and 2 falling below the limit of quantitation (LOQ) were set to zero. Mean concentration values and standard deviation (SD) were calculated at each timepoints of the pharmacokinetic study (n=4 animals/timepoint). The following pharmacokinetic parameters were calculated: area under the plasma concentration versus time curve from time zero to the last measurable concentration time point (AUC₀₋ₜ), area under the plasma concentration versus time curve extrapolated to infinity (AUC_{inf}), maximum observed plasma concentration (Cₘₐₓ), time of maximum plasma concentration (tₘₐₓ), apparent first-order terminal elimination rate constant (kₑₗ), apparent first-order terminal elimination half-life will be calculated as 0.693/kel (t_{1/2}). The systemic clearance (CL) of Compound 1 after intravenous administration was calculated using Dose/AUCinf. Pharmacokinetic parameters were calculated using Kinetica™ 4.1.1 (InnaPhase Corporation, Philadelphia, Pa.).

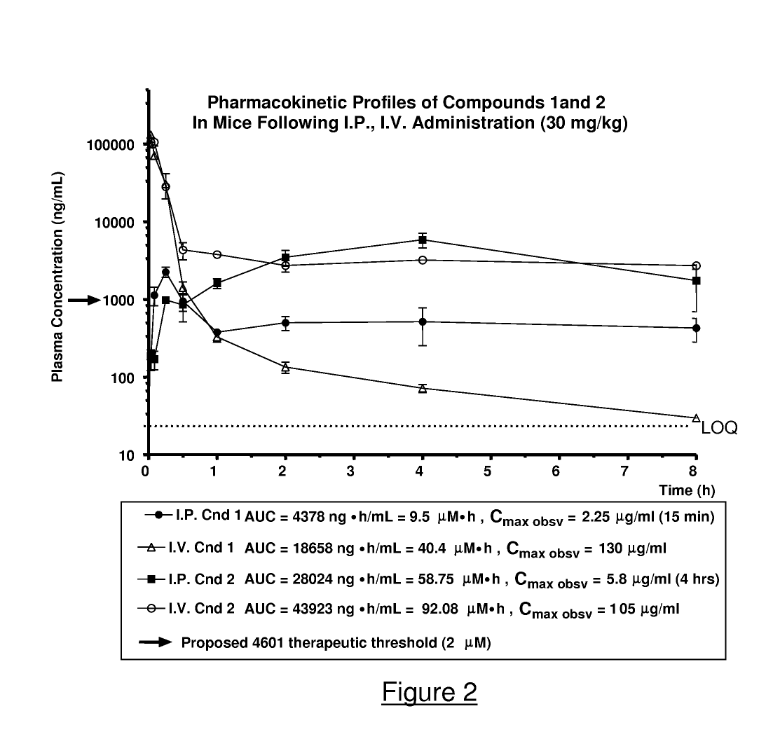

Mean plasma concentrations of Compound 2 following IV and IP administrations at 30 mg/kg, compared with Compound 1 via the same routes of administration, are presented in FIG. 2. When administered iv, Compound 2 had an AUC of 92.08 μM·h and an observed Cₘₐₓ of 105 μg/mL, compared to an AUC of 40.4 μM·h and an observed Cₘₐₓ of 130 μg/mL for Compound 1. When administered IP, Compound 2 had an AUC of 58.75 μM·h and an observed Cₘₐₓ of 5.8 μg/mL, compared to an AUC of 9.5 μM·h and an observed Cₘₐₓ of 2.25 μg/mL for Compound 1.Mean (±SD) plasma concentrations of Compound 1 following IV administration of a 30 mg/kg dose declined rapidly in a biexponential manner resulting in very short half lives (t_{1/2} α and β of 4.6 min and 2.56 h, respectively). The pharmacokinetics of Compound 1 following intraperitoneal administration, and Compound 2 following intraperitoneal and intravenous administration, showed a PK profile suggestive of slow release. With these routes of administration, the compound plasma concentration was sustained and maintained at therapeutically relevant levels for over 8 hours. Compound 2 showed a half life (t_{1/2}) of more than 40 hours following both IP and IV administrations.

Acute toxicity studies in CD-1 nu/nu mice for Compound 2, using the same formulation, gave an MTD≧50 mg/kg (ip, NOAEL: 30 mg/kg) and ≧100 mg/kg (iv, NOAEL: 75 mg/kg), with weight losses of about 7% for several days post-injection. Compound 1 had an MTD of 150 mg/kg when administered IV. Acute toxicity studies with Compound 46 gave an MTD of 30 mg/kg (ip).

### Example 4

### In Vitro Anticancer Activity of Compound 1

a) Human Tumor Cell Lines from the U.S. NCI Panel

A study measuring the in vitro cytotoxic activity of Compound 1 was first performed by the NCI (National Cancer Institute, U.S. National Institutes of Health, Bethesda, Md., USA) against a panel of human cancer cell lines. This screen utilizes 60 different human tumor cell lines, representing cancers of the blood, skin, lung, colon, brain, ovary, breast, prostate, and kidney. Further information regarding the NCI panel of human cancer lines can be obtained by following the links at the NCI world-wide website of the National Cancer Institute. The compound was sent and tested on three occasions (Mar. 31, 2003; Dec. 1, 2003; Mar. 27, 2007).

The results from the NCI in vitro screening indicate that Compound 1 has broad cytotoxic activity in the low micromolar range in the 60 different cell lines tested. The compound showed activity in vitro against leukemia (GI₅₀ range=0.9-5.0 μM), non-small cell lung carcinoma (GI₅₀ range=1.9-10.8 μM), melanoma (GI₅₀ range=1.8-8.1 μM), prostate carcinoma (GI₅₀ of 3.5-9.3 μM), breast carcinoma (GI₅₀ range=1.4-16.3 μM), ovarian carcinoma (GI₅₀ range=2.5-6.2 μM), renal carcinoma (GI₅₀ range=2.9-14.5 μM), colon carcinoma (GI₅₀ range=3.0-17.3 μM) and CNS (glioblastoma, GI₅₀ range=2.0-6.5 μM) tumor cell lines.

Following the “flat” pattern of activity of Compound 1 across the cell lines tested, no significant correlation was observed using the COMPARE alogorithm.

### b) Human and Animal Glioma Cell Lines (IC50)

The cytotoxic activity of Compound 1 was further evaluated using a panel of brain tumor cell lines. This study was performed in collaboration with INSERM (Grenoble, France). Tumor cells (5,000 to 10,000 cells per well depending on their doubling time) were plated in 96-well flat-bottom plates and incubated for 24 hours before treatment. Tumor cells were then incubated for 96 hours with seven different concentrations of Compound 1: 10, 1, 0.5, 0.1, 0.5, 0.01, and 0.001 μM. The in vitro cytotoxic activity was determined by a standard MTT assay. Results in Table 6 are expressed as the concentration of drug that inhibits 50% of the cell growth (IC₅₀) as compared to non-treated control cells.

TABLE 6

| Cell line | Type | Origin | IC₅₀ at 96h (μM) |
|---|---|---|---|
| 9L | Gliosarcoma | Rat | 8.3 ± 3.8 (n = 4) |
| GHD | Astrocytoma | Human | 6.5 ± 2.9 (n = 8) |
| U 373 | Astrocytoma | Human | 3.8 ± 1.4 (n = 4) |
| GL26 | Glioblastoma | Human | 8.9 ± 1.1 (n = 4) |
| C6 | Glioblastoma | Rat | 4.3 ± 2.3 (n = 5) |
| DN | Oligodendroglioma | Human | 3.0 ± 0.7 (n = 4) |
| GHA | Oligodendroglioma | Human | 1.6 ± 0.7 (n = 10) |

The IC₅₀ values of Compound 1 against different representative types of brain tumor cell lines were similar, ranging from 1.6 to 8.9 μM. These results confirmed the activity of TLN-4601 against different brain cancer cell lines including a rat glioblastoma C6 cell line, which is the most malignant form of brain cancer, type IV glioblastoma multiform.

### Example 5

### Benzodiazepine Receptor Binding Assays

As Compound 1 was isolated from structural prediction through genetic analysis and activity identified through in vitro cytotoxic assays, its molecular target(s) were unknown at the time of discovery. Based on the structural characteristics of TLN-4601, we first investigated its binding affinity to the central (GABA_{A}; CBR;) and peripheral (PBR) benzodiazepine receptors. The effect of TLN-4601 on CBR (GABA_{A}) and PBR was initially evaluated in a radioligand-binding assay at MDS Pharma Services (Taipei, Tawain). CBR and PBR were obtained from rat brain and heart membrane-fractions, respectively. Displacement assays were done in the presence of 1 nM [³H]-Flunitrazepam (CBR; GABA_{A}) or 0.3 nM of [³H]-PK11195 (PBR). TLN-4601 was tested at 0.01, 0.1, 0.5, 1, 5 and 10 μM. Non-specific binding was estimated in the presence of 10 μM diazepam (CBR) or 100 μM dipyrimadole (PBR) and assays were performed according to previous described methods (Damm et al Res Commun Chem Pathol Pharmacol 22, pp 597-600; Le Fur et al (1983) Life Science 33, pp 449-57). Results obtained from these binding studies indicated that TLN-4601 did not bind the CBR (IC₅₀>10 μM) while the binding affinity for the PBR was ˜0.3 μM. The binding affinity of TLN-4601 to the PBR is similar to the concentration required to inhibit cell proliferation (1 to 10 μM, depending on cell lines). This contrats with current specific PBR ligands, which bind the PBR with nanomolar affinity yet their effect on cell proliferation, is in the micromolar range.

### a) Establishment of a PBR Binding Assay:

In order to screen analogs of Compound 1 for PBR binding affinity, a

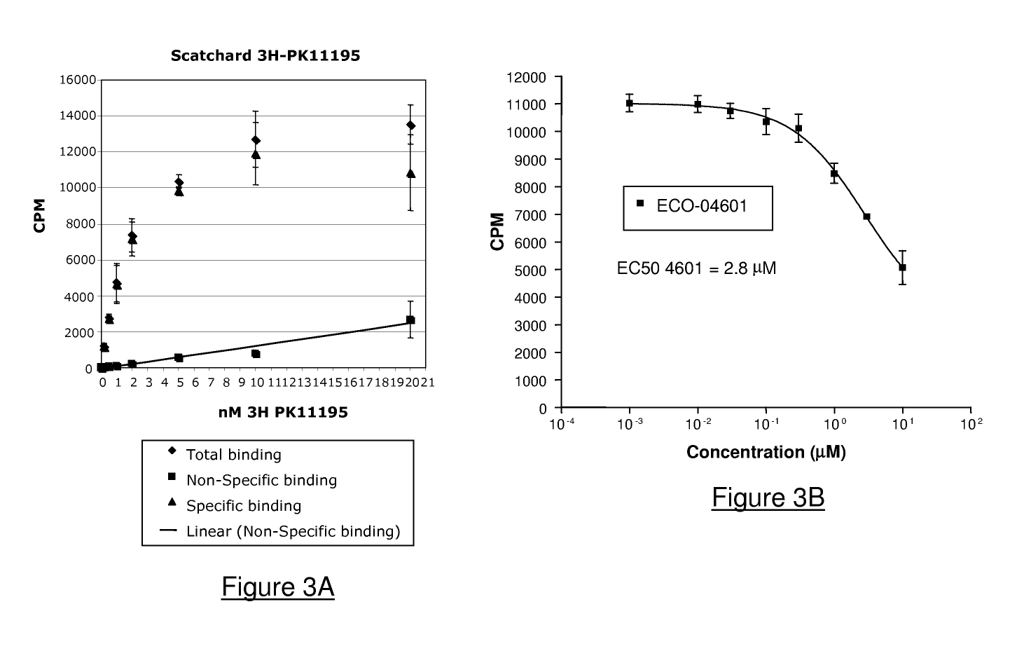

PBR binding assay was implemented at Thallion Pharmaceuticals. Hearts obtained from 3 Sprague Dawley rats were homogenized in 20 volumes of ice-cold 50 mM Tris-HCl, pH 7.5. After two centrifugations at 1500 g for 10 minutes at 4° C., the supernatant was centrifuged at 48000 g for 20 minutes at 4° C. The resulting pellet was resuspended in 50 mM Tris-HCl pH 7.5 and protein concentration was estimated by the Bradford colorimetric staining method using BSA as the standard. For equilibrium binding parameters determination, [³H]PK11195 (specific activity, 84.8 Ci/mmol) binding assays were conducted in a final volume of 300 μl of PBR-binding buffer (50 mM Tris-HCl, pH 7.5 and 10 mM MgCl₂) containing the enriched mitochondria membrane preparation (25 μg of protein) and 0.2 nM to 20 nM of [³H]PK11195. In parallel, non-specific binding was measured with the presence of 20 μM cold PK11195. Samples were distributed onto 96-well GF/B filtration plates and incubated for 60 minutes at 25° C. and then washed once with PBR-binding buffer. Filters were punched out and radioactivity measured on a Perkin Elmer TriCarb 2800 Scintillation counter (Janssen et at (1999) J Pharmaceutical and Biomedical Analysis 20, pp 753-761). Scatchard plot analysis of the data by the GraphPad Prism 3.0 software determined a Kd of 1.37 nM for [3H]PK11195 (FIG. 3A).

### b) PBR Binding Affinity of Compound 1:

Binding affinity of TLN-4601 for the PBR was evaluated using the experimental conditions above. For this assay, 25 μg of enriched mitochondrial membrane fraction was incubated with a fixed concentration of [3H]PK11195 (0.5 nM; specific activity 84.8 Ci/mmol) and increasing concentrations of TLN-4601 (0.01 μM t0 10 μM). From the results presented in FIG. 3B, an EC50 of TLN-4601 was determined by the GraphPad Prism 3.0 software to be 2.8 μM, leading to a calculated a Ki value of about 1.4 μM (using the formula of Ki=EC50/(1+[ligand]/Kd), where the [ligand]=1.6 nM).

c) TLN-4601 Concentrations in Tumors and Brains Obtained from Rat C6 Orthotopic Brain Tumors:

i) Cell Culture and Spheroid Preparation

Rat C6 glioma cells were purchased from the American Type Culture Collection (Manessa, Va.) and grown in Dulbecco's modified Eagle's medium (DMEM) supplemented with 10% FBS, 125 U/mL penicillin G, 125 μg/mL streptomycin sulfate, and 2.2 μg/mL amphotericin B (Fungizone). All culture reagents were obtained from Gibco BRL (Invitrogen Burlington, ON, Canada). Cultures were grown in monolayers and maintained at 37° C. in a humidified atmosphere of 5% CO₂. Upon reaching confluency, spheroids were prepared using the hanging drop method previously described by Del Duca et al. ((2004) J. Neurooncol 67, p 295). Briefly, 20 μl drops of DMEM containing 15,000 cells each were suspended from the lids of culture dishes and the resulting aggregates were transferred to culture dishes base-coated with agar after 72 hours. The resulting spheroids were adequate for in vivo implantation after 48 hours of incubation on agar.

ii) Surgical Implantation of Rat C6 Tumor Cells

Male, Sprague-Dawley rats (250-300 g) (Charles River Canada, St Constant, QC) were anesthetized with 50 mg/kg ketamine and 10 mg/kg xylazine. The right cortical surface in the parietal-occipital region was exposed by craniectomy using a high-powered drill (DREMEL, USA) and the underlying dura and its vessels were carefully removed under a surgical microscope. A piece of the cortex was removed to expose the underlying white matter and a single speroid containing rat C6 tumor cells was placed into the surgical defect. The craniectomy was covered with bone wax (Ethicon, Peterborough, Canada) and the overlying skin sutured.

iii) In Civo 11C-PK11195 PET Imaging in Rats

(R)-1-(2-chlorophenyl)-N-methyl-N-(1-methyl-propyl)-3-isoquinoline carboxamide (R N-desmethyl PK11195), the precursor for the radioisotope-labeled (R)-PK11195, was purchased from ABX (Radeburg, Germany). The synthesis of ¹¹C-(R)-PK11195 was accomplished by a modification of the method of Camsonne et al. (J. Label. Compd. Radiopharm., 21: 985-991, 1984). In vivo PET studies were performed 14 days post tumor implantation. PET imaging studies were performed while the animals were anesthetized and placed in the supine position on the bed and at the center of the FOV of the CTI Concorde R4 microPET scanner (Siemens/CTI Concorde, Knoxville, Tenn.). Each dynamic PET study lasted 60 min and was initiated with an IV bolus administration of 11C-PK11195 (7.1-12.7 MBq) radioligand via the tail vein. Receptor occupancy studies were performed by acquisition of 11C-PK11195 images prior to and during TLN-4601 treatment over 60 minutes. TLN-4601 was administered by a bolus IV infusion (30 mg/kg) followed by continuous IV infusion (5 mg/h/kg) lasting through the dynamic scan. Attenuation correction factors, for each 6 rats, were determined using a 10 minute 57 Co transmission scan acquired immediately prior to the dynamic scan. In addition, all images were scatter corrected.

Following completion of in vivo studies, animals were sacrificed by anesthetic overdose and decapitated. Brain, tumor, and liver were snap-frozen in liquid nitrogen and stored frozen (−70° C.±10° C.). For blood samples, each blood sample was collected into a K2-EDTA tube and kept on wet ice for a maximum of 30 minutes. Blood samples were centrifuged under refrigeration (2 to 8° C.) for 10 minutes at 1,500 g (RCF). A volume of 25 μL of aqueous 4% w/v L-ascorbic acid was added to a volume of 225 μL of rat plasma in a clean tube, and the samples were thoroughly mixed by inversion. A volume of 125 uL of the resulting mixture was transferred to a separate tube for bioanalysis, while the remaining mixture was maintained as a backup, and both the bioanalysis and back-up portions were frozen on dry ice and stored frozen (−70° C.±10° C.).

iv) Sample Extractions and HPLC/MS/MS Analysis

Rat plasma and tissue samples were extracted with 9 volumes of acetone containing 100 ng/mL of the internal standard (Compound 2) and analysed by HPLC/MS/MS as described in Gourdeau et al (Cancer Chemother Pharmacol vol 61 pp. 911-921).

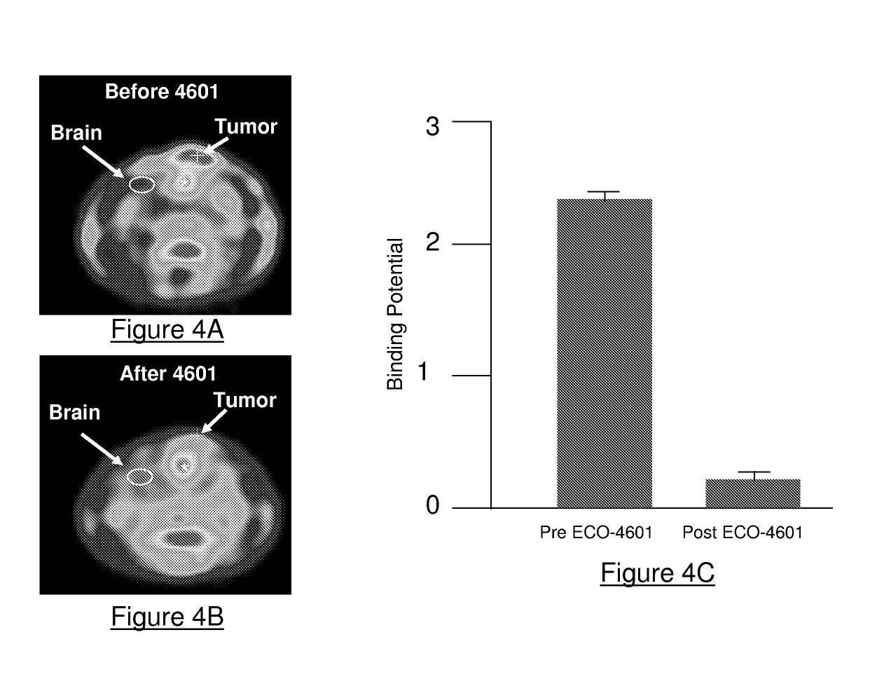

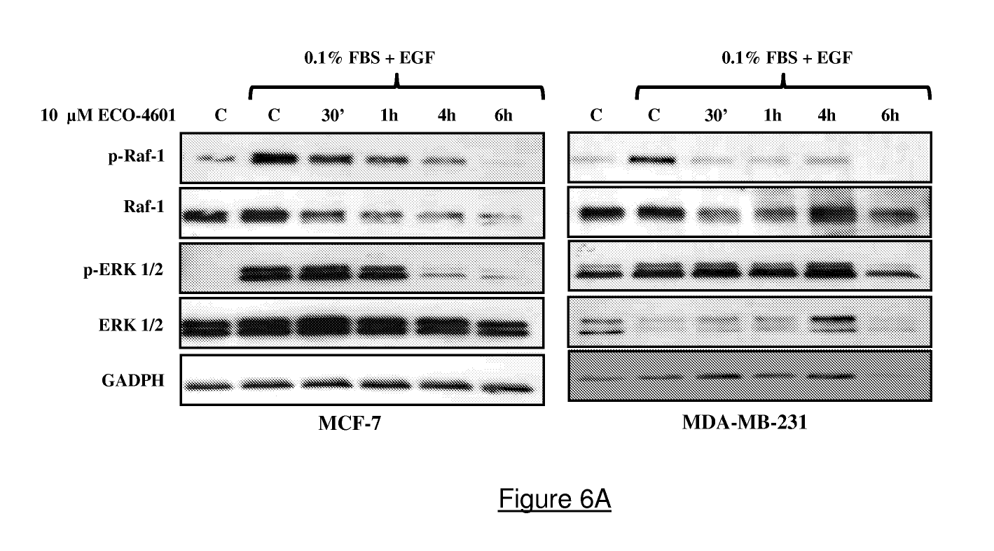

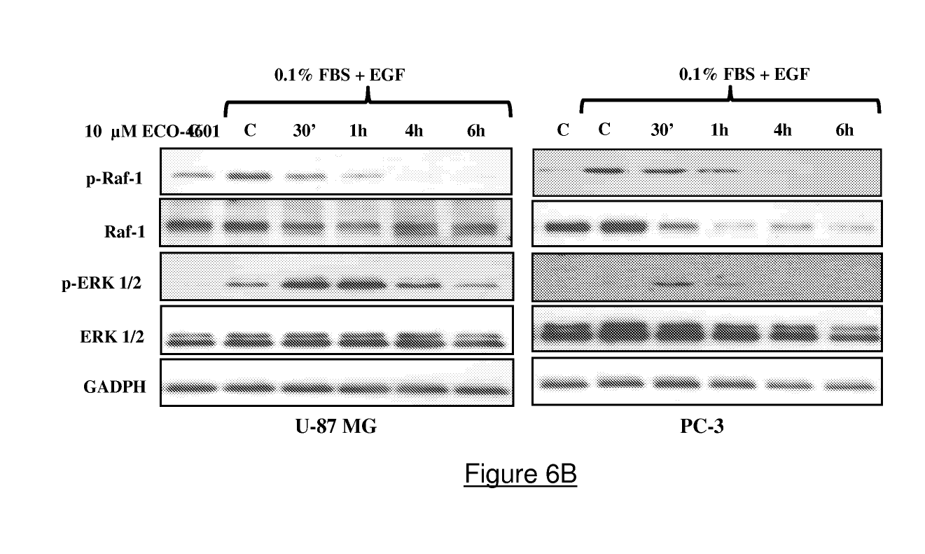

Representative ¹¹C-(R)-PK11195 microPET images from the CIV study are shown in FIGS. 4A and B, which on a comparison of the image presented in FIG. 6B (after administration of TLN-4601) to the image presented in FIG. 4A (before administration of TLN-4601) shows a significant blocking of the radiotracer from the peripheral part of the tumor (area of specific binding) following CIV administration of TLN-4601. An area of non-specific binding is indicated by an asterisk (*) and was considered as a likely necrotic area.

To determine a mean tumor binding potential (B.P.) (baseline) and the mean B.P., the simplified reference tissue method was utilized comparing the ratio of tumor to normal brain. As a result, mean tumor binding potential (B.P.) (baseline) was calculated to be 2.19±0.16 (mean±SEM) and the mean B.P. (TLN-4601) was calculated to be 0.14±0.13 (mean±SEM). Graphically, results from the mean B.P calculations from the competition binding studies are shown in FIG. 4C, where it can be observed that after the CIV infusion of TLN-4601, the PBR occupancy for ¹¹C-(R)-PK11195 binding was reduced by an average of 91.67% (P<0.001, n=6).

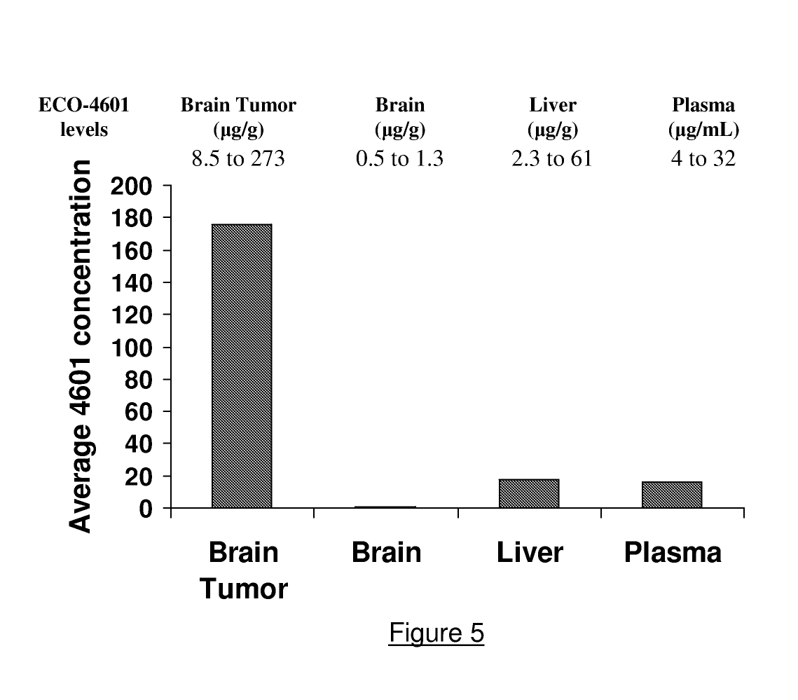

The studies presented in Example 5 clearly demonstrate that Compound 1 binds the PBR both in vitro and in vivo. Furthermore, this binding affinity results in preferential accumulation of Compound 1 in tumor tissue compared to normal tissue as demonstrated by the 10 to 200 fold higher levels of Compound 1 observed in orthotopic rat brain tumors compared with the rest of the brain area (normal tissue). Compound 1 accumulation in the tumor (176 μg/ml) was also significant compared to liver (24.8 μg/g; 7-fold) and plasma (16.2 μg/g; 11-fold) (FIG. 5).

### Example 6

### Effect of Compound 1 on the RAS-MAPK Pathway

Related to its farnesylated moiety, the effect of TLN-4601 was assessed on the RAS signaling pathway. The RAS-MAPK signaling pathway has long been viewed as an attractive pathway for anticancer therapies, based on its central role in regulating the growth and survival of cells from a broad spectrum of human tumors (Downward 2003 Nature Reviews Cancer, 3:11-22; Sebolt-Leopoldd and Herrera 2004 Nature Reviews Cancer 4: 937-947).

The effect of TLN-4601 on downsteam events of RAS signaling was examined by monitoring the phosphorylation levels of Raf-1 and ERK1/2 by Western blot analysis. To study the effect of TLN-4601 on the RAS-MAPK signaling pathway, exponentially growing cells (human breast MCF-7 tumor cells, human breast MDA-MB-231 tumor cells, human glioma U 87-MG tumor cells and human prostate PC-3 tumor cells) were seeded onto 60 mm tissue culture dishes (0.5 to 0.8×10⁶ cells per dish) for 24 h. The media was removed and cells were treated with 10 μM TLN-4601 in culture medium supplemented with 0.1% FBS for 30 min, 1 h, 4 h and 6 h, and subsequently exposed to EGF at 50 ng/mL for 10 min at 37° C. Control plates consisted of cells incubated in culture medium containing 0.1% FBS and 0.05% DMSO (vehicle) with or without EGF stimulation. At the end of each treatment, media was removed and cells rinsed with ice-cold PBS. Cells were then harvested by scraping and cell pellets were lysed in ice-cold RIPA buffer for 20 minutes on ice. Unsolubilized material was pelleted and discarded. The protein concentration of each lysate was quantified using the Bio-Rad protein assay (Bio-Rad Laboratories). Equivalent amounts of protein (20-30 ug protein) were separated on 10% or 12% SDS-PAGE under reducing conditions, transferred onto nitrocellulose membranes (0.2 μm; Bio-Rad Laboratories) and blotted as above with phospho-c-Raf (Ser338) and c-Raf (Cell Signaling Technology Inc., Boston, Mass.), phospho-p44/42 (Thr202/Tyr204, p-ERK1/2) and p44/42 (ERK1/2) MAP Kinases (Cell Signaling Technology Inc.) and GAPDH (SantaCruz Biotechnology Inc.).

A strong inhibition of EGF-induced phosphorylation of Raf-1 and ERK1/2 was observed (FIGS. 6A and 6B). This effect was time dependent with complete inhibition of protein phosphorylation within 6 h. It was also noted that TLN-4601 not only inhibited Raf-1 phosphorylation, but also caused a decrease in the amount of total Raf-1.

Unlike current RAS signalling pathway inhibitors, TLN-4601 is not a direct kinase inhibitor. This was documented by evaluating the effect of TLN-4601 on human EGFR, c-RAF, MEK1, MAPK1 (ERK1) and MAPK2 (ERK2) kinase-activity (Upstate Kinase Profiler™ Service; Dundee, UK). TLN-4601 was tested at 0.5 μM and 5 μM in a final volume of 25 μL according to standard protocols developed by Upstate Ltd. Briefly, purified recombinant human enzymes were incubated with 25 mM Tris pH 7.5 containing EGTA, a specific substrate and γ-³²P-ATP. The reaction was initiated with MgATP mix and incubated for 40 minutes at RT. The reaction was stopped by the addition of 5 μL of a 3% phosphoric acid solution; aliquots were spotted on filters and counted. Detailed procedures are available on the Millipore Upstate website. Results of the direct inhibition of kinase activities by TLN-4601, summarized in Table 7, indicate that TLN-4601 does not directly inhibit EGFR, c-Raf, MEK1, ERK1 or ERK2 kinase activities.

TABLE 7

| | Kinase Activity* (%) ± SD | |
|---|---|---|
| | TLN-4601 | TLN-4601 |
| Kinases | (0.5 μM) | (5 μM) |
| EGFR | 128 ± 6 | 127 ± 9 |
| c-Raf | 114 ± 12 | 94 ± 2 |
| MEK1 | 106 ± 2 | 98 ± 1 |
| MAPK1 (ERK1) | 97 ± 2 | 73 ± 3 |
| MAPK2 (ERK2) | 116 ± 1 | 110 ± 1 |

*Data is expressed as the percentage of enzyme activity in the presence of TLN-4601 over
that of the positive control. Results are the mean of 2 separate experiments ± SD.

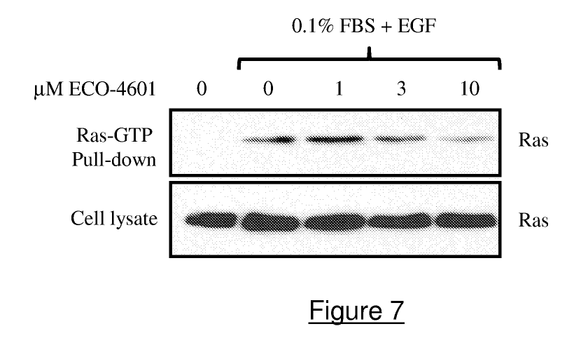

Following EGF induction, RAS is activated by a nucleotide exchange reaction that removes GDP and replaces it with GTP. Physiological levels of total cellular GTP-bound RAS can be detected with pull-down assays. MCF-7 cells were treated with increasing concentrations of TLN-4601 for 6 h and the RAS-MAPK signalling pathway was then induced with EGF. After a 5 min induction period, cells were lysed and incubated with a recombinant fusion protein that contains the isolated RAS Binding Domain of c-Raf-1 fused the gluthathione-S-transferese (GST; designated GST-Raf-RBD). The presence of RAS in the GST-Raf-RBD protein complex is resolved by western blotting. As expected, after EGF induction, an increase of RAS-GTP was observed. Interestingly, treatment of MCF-7 cells with TLN-4601 prevented EGF from activating RAS (FIG. 7).

### Example 7

### Assays of Dibenzodiazepinone Analogues and Derivatives

### a) Growth Inhibitory Assays:

Growth inhibitory activity of TLN-4601 (Compound 1) and other dibenzodiazepinone analogs was evaluated on a panel of 4 human tumor cell lines: the human uterine sarcoma MES-SA and its doxorubicin-resistant P-glycoprotein over-expressing variant, MES-SA/DX5 as well as non-aggressive and highly aggressive human breast cell lines, MCF-7 and MDA-MB-231, respectively. These four cell lines were obtained from the American Type Culture Collection (Manassas, Va.) and cultured in RPMI plus 10% fetal bovine serum (FBS) and maintained at 37° C. with 5% CO₂.

Exponentially growing cells (5,000 cells per well time; cell number determined with a hemocytometer) were seeded in 96-well flat-bottom plates and allowed to grow overnight. Cells were then incubated for 72 hours with three different concentrations of TLN-4601 or analogs: 30, 10, and 3 μM. The in vitro growth inhibitory activity was determined by a commercial MTT assay. All measurements were done in quadruplicate and each experiment was performed 2-3 times. Results are expressed as treated over control and the % of growth inhibition obtained at 10 μM is presented in Table 8. The lower the value, the more cytotoxic is the compound.

TABLE 8

| | % T/C at 10 μM | | | |
|---|---|---|---|---|
| Compounds | MES-SA | MES-SA/DX5 | MCF-7 | MDA-MB-231 |
| TLN-4601 | 78 | 100 | 44 | 38 |
| (Compound 1) | | | | |
| ECO-4625 | 80 | 83 | 28 | 68 |
| (Compound 97) | | | | |
| ECO-4657 | 28 | 24 | 50 | 55 |
| (Compound 99) | | | | |
| 4687 | 18 | 86 | 38 | 43 |
| (Compound 100) | | | | |

The data indicate that TLN-4601 and at least certain analogs of TLN-4601 are potent at inhibiting cell growth. This inhibition occurs in highly aggressive tumor cell lines and for some compounds in cells that are multidrug resistant (MES- to SA/5DX).

### (b) PBR Binding Assay

The effect of TLN-4601 and analogs on the peripheral benzodiazepine receptor (PBR) was evaluated in a radioligand-binding assay, implemented in house and described above. The data obtained is presented in Table 9.

TABLE 9

| Compounds | PBR Binding IC₅₀ (μM) |
|---|---|
| 4601 (Compound 1) | 2.7 |
| 4625 (Compound 97) | 2.6 |
| 4657 (Compound 99) | 0.01 |
| 4687 (Compound 100) | 0.01 |

These data indicate that TLN-4601 and analogs bind the PBR.

### (e) ERK Phosphorylation ELISA Assay

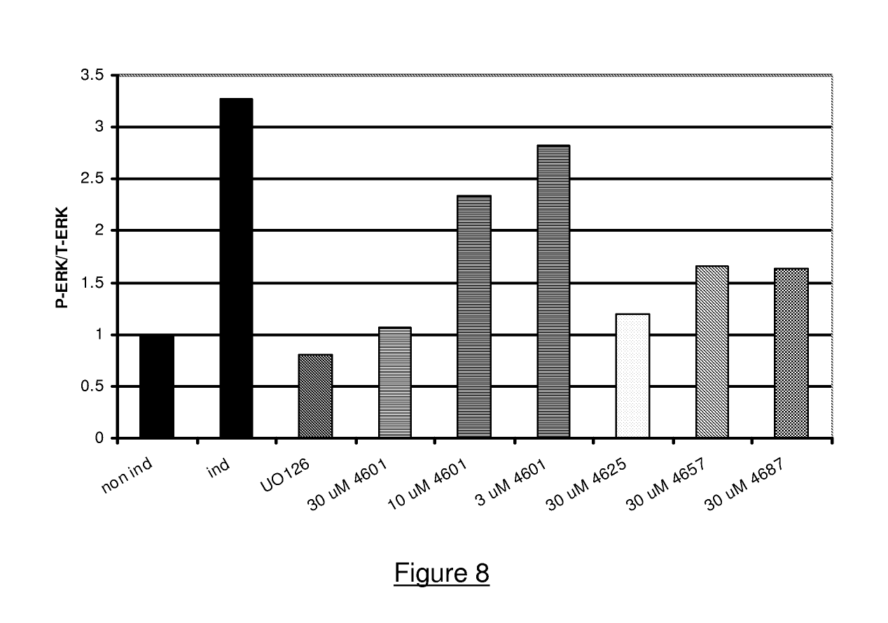

Human breast tumor MCF-7 cells were plated in 96-well culture plates (10,000 cells per well) in RPMI containing 10% FBS. After an overnight incubation, the medium is changed to low serum conditions (RPMI containing 0.1% FBS) for 18 h. Cells were then treated with TLN-4601 or selected analogs for 6 hours and then stimulated by the addition of EGF (100 ng/mL for 5 min) to induce the MAPK pathway. UO126 is a commercial inhibitor (Promega, Madison, Wis.) of mitogen-activated protein kinase kinase (MEK1/ERK). Following stimulation, cells were rapidly fixed, which preserved activation-specific protein modifications. Each well was then incubated with an antibody specific for Phospho-ERK or total ERK. After an one-hour incubation and several washes, cells were incubated with a secondary HRP-conjugated antibody followed by a developing solution that provided a colorimetric readout that is quantitative and reproducible. The Fast Activated Cell-based ELISA (FACE™) is commercially available (Active Motif, Carlsbad, Calif.). The data obtained with Compound 1 and selected analogs clearly demonstrate that they all inhibit the RAS-MAPK signaling pathway shown by their inhibition of phospho-ERK in the FACE ELISA assay (FIG. 8).

### Example 8

### Inhibition of Basal and EGF-Induced Migration of Glioma Cells Harboring WT, Amplified and Mutated EGFRs

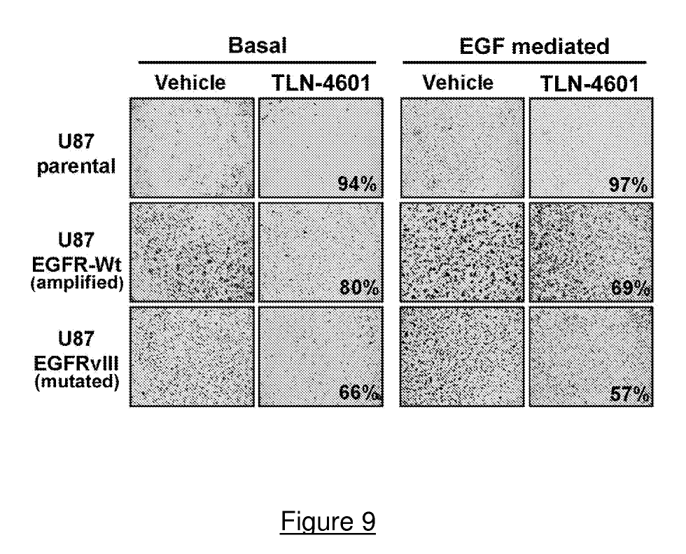

The ability of Compound 1 to inhibit or effect a reduction of basal and EGF-induced cell migration in a glioma cell model system was tested as follows. Exponentially growing cells (U87 parental, U87 transfected with EGFR-WT, and U87 transfected with mutated EGFR VIII) (5×10⁵) were dispersed onto 1 mg/ml gelatin/PBS-coated chemotaxis filters (Costar; 8-μm pore size) within Boyden chamber inserts. Migration proceeded for 18 h at 37° C. in 5% CO₂ in the presence or absence of 5 μM of Compound 1 (TLN-4601). Cells that had migrated to the lower surface of the filters were fixed with 10% formalin phosphate, colored with 0.1% crystal violet/20% MeOH and counted by microscopic examination. The percent inhibition of cell migration after treatment with Compound 1 vs vehicle (0.1% DMSO) treated cells is shown in the attached FIG. 9.

As can be see from the results presented in FIG. 9, over-expression of WT EGFR (mimicking amplified) or EGFRvIII (mutated) resulted in a significant increase in cell migration verus control (U87 parental), which was further increased by the addition of EGF (third column). Furthermore, as can be observed from the micrographs presented under each of the columns indicated as “TLN-4601” in FIG. 9, Compound 1 (TLN-4601) significantly inhibited both basal and EGF-mediated cell migration of the highly invasive glioma cell lines.

### Example 9

### Inhibition of the RAS-MAPK Signaling Pathway in Glioma Cells WT, Amplified and Mutated EGFRs

Exponentially growing cells (U87 parental, U87 transfected with EGFR-WT, and U87 transfected with mutated EGFR VIII) were plated onto 100 mm³ dishes in DMEM containing 10% FBS. 24 h after plating, the media was removed and cells were treated with 5 μM of Compound 1 (TLN-4601) for 18 h in media containing 0.1% FBS. Cells were then stimulated for 1 min with100 ng/ml EGF and harvested. Western blots were performed (according to standard protocols as known in the art) and analyzed for p-EGFR, Raf-1, p-ERK, ERK and AKT using specific commercial antibodies.

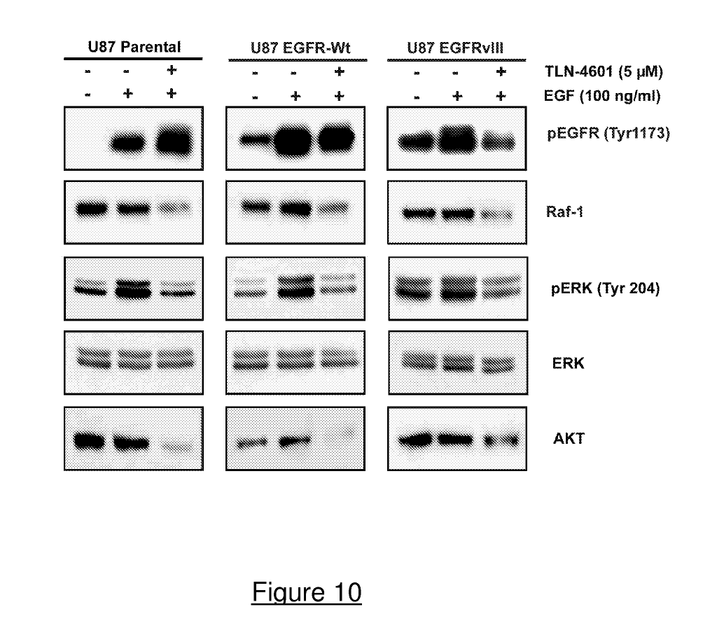

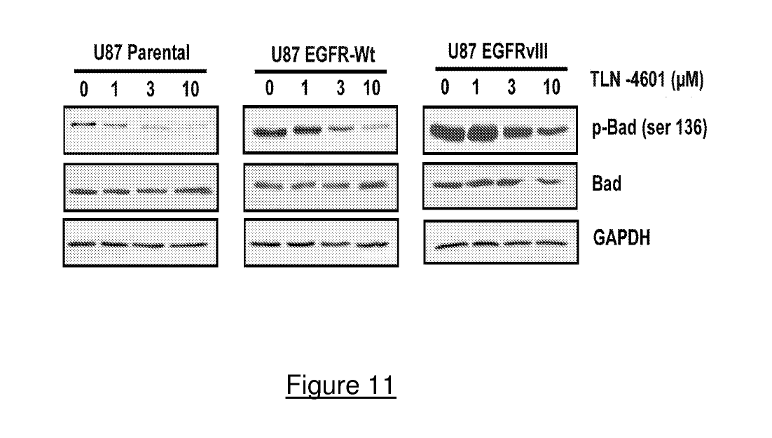

As can be see from the results presented in FIG. 10, while EGFR is not phosphorylated under basal conditions in the U87 MG parental cell line, it is phosphorylated in cells transfected with WT (mimicking EGFR amplification) and mutated (viii) EGFRs without need for addition of EGF. Furthermore, EGF stimulated receptor phosphorylation, and this stimulation was not affected by the presence of Compound 1. Finally, exposure of the cells to Compound 1 as described above resulted in a decrease of total Raf-1 and decreased EGF induction of p-ERK as well as a reduction in the cell survival Pi3K pathway enzyme AKT.

### Example 10

### Reduction of AKT Signaling by Compound 1

To confirm the ability of Compound 1 to effect a reduction in AKT signaling in the highly invasive glioma cell lines, thereby leading to an induction of apoptosis in the treated cells, the following experiment was conducted. Exponentially growing cells (U87 parental, U87 transfected with EGFR-WT, and U87 transfected with mutated EGFR VIII) were plated onto 100 mm³ dishes in DMEM containing 10% FBS. Twenty-four hours after plating, the media was removed and cells were re-fed with serum-free DMEM and increasing concentrations of Compound 1 (TLN-4601) for 18 h. Cells were harvested and Western blots were performed (according to standard protocols as known in the art) and analyzed for p-Bad (indicating functional AKT signaling) and Bad using specific commercial antibodies. GAPDH was used as a loading control.

As can be seen from the results presented in FIG. 11, exposure of the glioma cell lines to Compound 1 resulted in a dose-dependent decrease of p-Bad, thus indicating that Compound 1 can effect a reduction in AKT signaling and cell survival in the highly invasive glioma cell lines.

### Example 11

### Induction by Compound 1 of Casepase Activation and PARP Cleavage in Glioma Cells Harboring WT, Amplified and Mutated EGFRs

To further assess the ability of Compound 1 to stimulate apoptotic cell death, along with effecting an inhibition or reduction in cell migration, in a highly invasive glioma cell line, the following experiments were conducted.

Firstly, cells (U87 parental, U87 transfected with EGFR-WT, and U87 transfected with mutated EGFR VIII) were plated in 6 well plates in DMEM containing 10% FBS. The following day, the plated cells were treated with increasing concentrations of Compound 1 (TLN-4601) in serum-free medium. After an 18 h incubation period in the presence of Compound 1, the treated cells were measured for caspase-3 activity using a commercial kit.

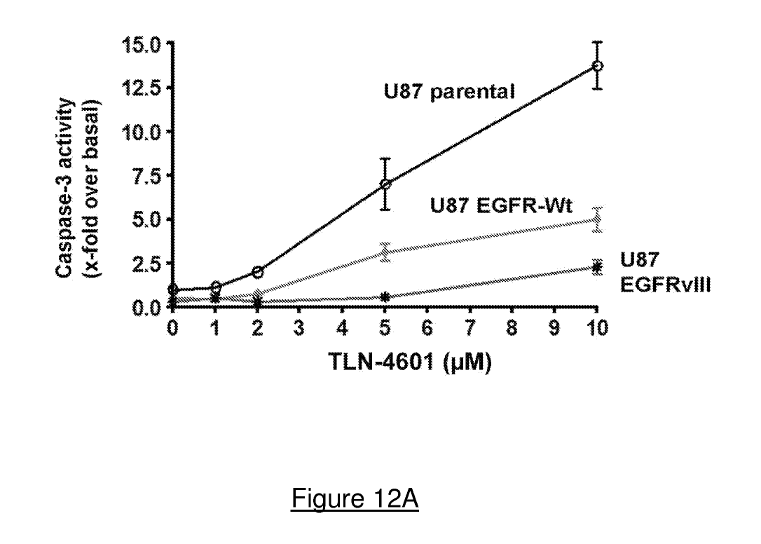

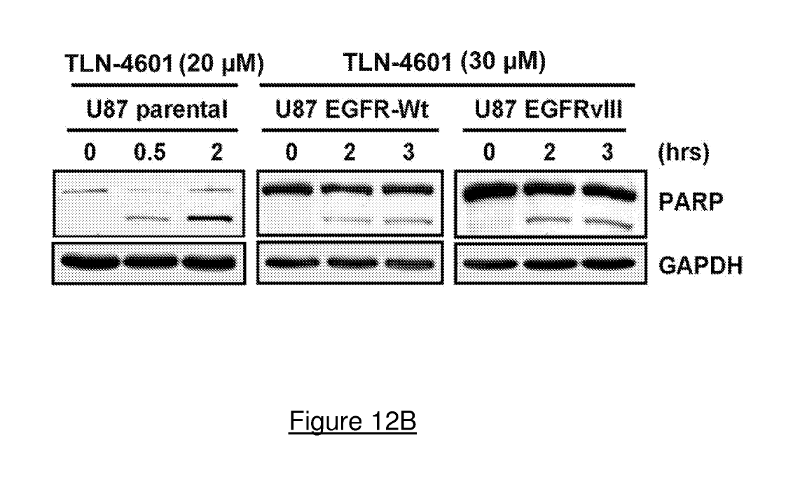

As can be seen from the results presented in FIG. 12A, a significant increase (approximately 15-fold) in caspase-3 activation was observed in the U87 parental cell line after incubation with Compound 1, thus indicating that Compound 1 can induce a cytotoxic or apoptotic effect in this cell line. As well, caspase-3 activation was also detected in glioma cells over-expressing WT and mutated EGFRs, although the degree (approximately 2 to 4-fold) of activation in these cell lines did not occur to as great a level as compared to the parental U87 MG cell line.

Secondly, exponentially growing cells (U87 parental, U87 transfected with EGFR-WT, and U87 transfected with mutated EGFR VIII) were plated onto 100 mm³ dishes in DMEM containing 10% FBS. Twenty-four hours after plating, the media was removed and the plated cells were re-fed with DMEM containing 0.1% FBS and 20 μM of Compound 1 (U87 MG) or 30 μM of Compound 1 (U87 EGFR-WT and U87 EGFRvIII) at different times. Cells were harvested and Western blotted (according to standard protocols as known in the art) and analyzed for PARP and GAPDH.

As can be seen by the results presented in FIG. 12B, exposure to Compound 1 resulted in PARP cleavage in each of the three cell lines, thus indicating that Compound 1 has an apoptotic cell death inducing effect on these highly invasive tumor cells.

### Example 12

### Effect of Compound 1 on Migration of Normal Endothelial Cells

Migration of endothelial cells is a key event in angiogenesis. In vitro, this process can be reconstituted by plating cells onto gelatin-coated filters inserted in modified Boyden chemotactic chambers (Transwell, 8 μm pore size; Corning-Costar, Acton, Mass.). The effect of Compound 1 on a normal endothelial cell's capacity to migrate was monitored by observing the number of cells that migrated in comparison to untreated control cells using a chemotactic assay. Cells [Human Microvascular Endothelial Cells from Brain—(HMVEC-B)] were pretreated with 5 μM TLN-4601 for 18 hours, then dislodged from the flasks by trypsinization, washed and resuspended in serum-free media. Dead cells were removed through a simple low-speed centrifugation, and only live cells were seeded in the Boyden chamber as described further below, and as such, the intrinsic capacity of live TLN-4601 pre-treated cells to migrate or respond to any of the chemotactic effectors enumerated below was measured. As a further measure to ensure that any effect that the pre-treatement with TLN-4601 would have would not be merely due to any cytotoxic activity of TLN-4601, pre-treated cells were, after treatment with TLN-4601, subjected to a Trypan Blue dye exclusion assay so as to ensure that only live cells were selected for seeding into the Boyden chambers.

Cells were placed onto gelatin-coated filters inserted in chambers and incubated at 37° C., 5% CO₂ for 30 min to allow adequate anchoring to the filters. The monolayers were then exposed to either to serum-free media or to media containing brain tumor-derived growth factors (conditioned media isolated from serum-starved U87 glioma cells) added within the lower compartment of the chambers. Cell migration was allowed to proceed for another 6 hours. Filters were then fixed in formalin phosphate solution, and stained with Crystal violet. The filter containing the migrated cells was quantified by microscopy to determine the average cell number/field of view (50×).

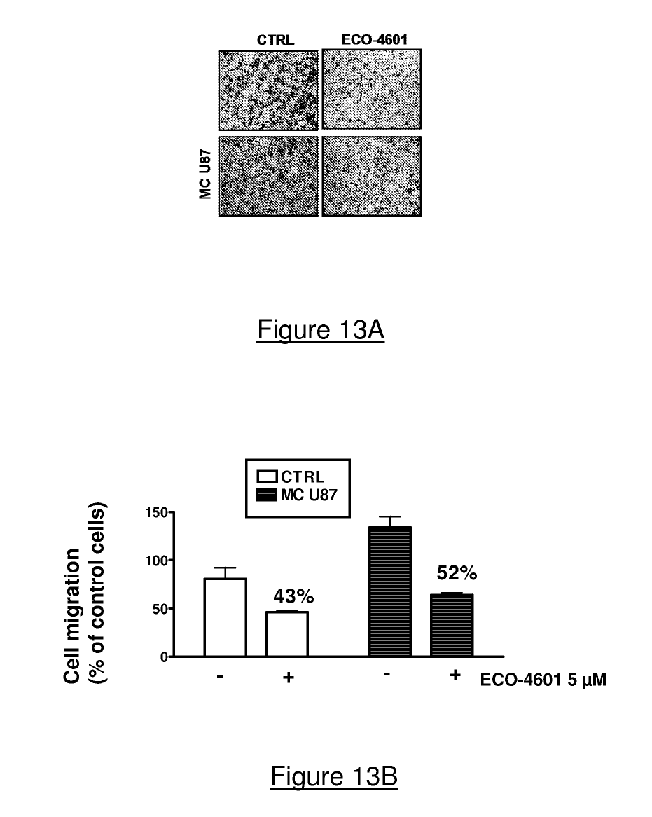

As can be seen from the results presented in FIG. 13A, both the basal (top row) and tumor-derived growth factors-induced migration (bottom row) were observed to be affected by treatment of the cells with Compound 1 (“ECO-4601”) versus control cells not pre-treated with Compound 1 (“CTRL”). Further, as can be seen from the results presented in FIG. 13B, both the basal (open bars) and tumor-derived growth factors-induced migration (solid bars) were significantly decreased by about 43%-52% by treatment of the cells with Compound 1 (“+ECO-4601”) when compared to the untreated cells (“−ECO-4601”).

### Example 13

### Effect of Compound 1 on Casepase 3 Induction in the Tumor and Vascular Endothelium Compartments

To test the effect of Compound 1 on caspase 3 activity in endothelial cells, the following experiment was conducted. HVMEC-B and U87 glioma cells were treated with increasing concentrations of Compound 1 (0-30 μM) in serum-free media for 18 hours. Fluorimetric caspase-3 activity assay was performed as follows: cells were grown to about 60% confluence in 6-well dishes and treated with increasing concentrations of Compound 1 for 18 hours. Cells were then collected and washed in ice-cold PBS pH 7.0. Cells were subsequently lysed in Apo-Alert lysis buffer (Clontech, Palo Alto, Calif.) for 1 hr at 4° C. and the lysates were clarified by centrifugation. Caspase-3 activity was determined by incubation with 50 μM caspase-3-specific fluorogenic peptide substrate acetyl-Asp-Glu-Val-Asp-7-amino-4-trifluoromethylcoumarin (Ac-DEVD-AFC) in 96-well plates. The release of AFC was monitored for at least 30 min at 37° C. on a fluorescence plate reader (Molecular Dynamics (Amersham Biosciences Inc, Sunnyvale, Calif.)) (λₑₓ=400 nm, λₑₘ=505 nm).

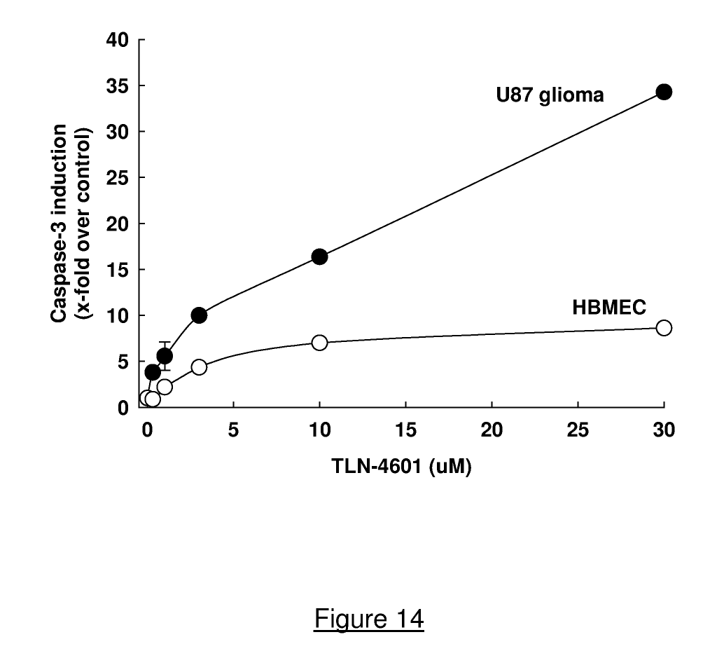

As can be seen from the results presented in FIG. 14, the experimental data (expressed as fold induction over untreated cells) indicated that the U87 glioma cells exhibited an approximately 2-3 fold greater caspase-3 activity as compared to normal human brain endothelial cells upon treatment of the cells with Compound 1.

### Example 14

### Effect of Compound 1 on Capillary-Like Structure Formation by Human Brain Endothelial Cells

Human brain microvascular endothelial cells (HBMEC) were characterized and generously provided by Dr Kwang Sik Kim from the John Hopkins University School of Medicine (Baltimore, Md.). These cells were positive for factor VIII-Rag, carbonic anhydrase IV and Ulex Europeus Agglutinin I; they took up fluorescently labelled, acetylated low-density lipoprotein and expressed gamma glutamyl transpeptidase, demonstrating their brain EC-specific phenotype. HBMEC were immortalized by transfection with simian virus 40 large T antigen and maintained their morphologic and functional characteristics for at least 30 passages. HBMEC were maintained in RPMI 1640 (Gibco, Burlington, ON) supplemented with 10% (v/v) inactive fetal bovine serum (iFBS) (HyClone Laboratories, Logan, Utah), 10% (v/v) NuSerum (BD Bioscience, Mountain View, Calif.), modified Eagle's medium nonessential amino acids (1%) and vitamins (1%) (Gibco), sodium pyruvate (1 mM) and EC growth supplement (30 μg/ml). Culture flasks were coated with 0.2% type-I collagen to support the growth of HBMEC monolayers. Cells were cultured at 37° C. under a humidified atmosphere containing 5% CO₂. All experiments were performed using passages 3 to 28.

To test the effect of Compound 1 on human brain endothelial cells to form capillary-like structures, an in vitro Matrigel™ (available from BD Biosciences, San Jose, Calif.) three-dimensional model assay was employed. The in vitro Matrigel three dimensional ECM model assay provides a physiologically relevant environment for studies of cell morphology, biochemical function, and gene expression in endothelial cells (EC) that can be modulated for instance by tumor growth factors or hypoxic culture conditions. Moreover, proteomic-based approaches to monitor levels of protein expression can also be achieved. When plated on Matrigel, EC have the ability to form capillary-like structures, and thus mimicking in vivo angiogenesis. The extent of capillary-like structures formation (density and size of structures) can be quantified by analysis of digitized images to determine the relative size and area covered by the tube-like network, using an image analysis software (Un-Scan-it, Empix Imaging, Mississauga, Ontario). HBMEC were trypsinised, counted and seeded on Matrigel. Adhesion to Matrigel was left to proceed for 30 minutes. Treatment with increasing concentrations of Compound 1 (0-10 μM) was then performed in serum-free media for 24 hours. The extent of capillary-like structure formation was then assessed afterwards.

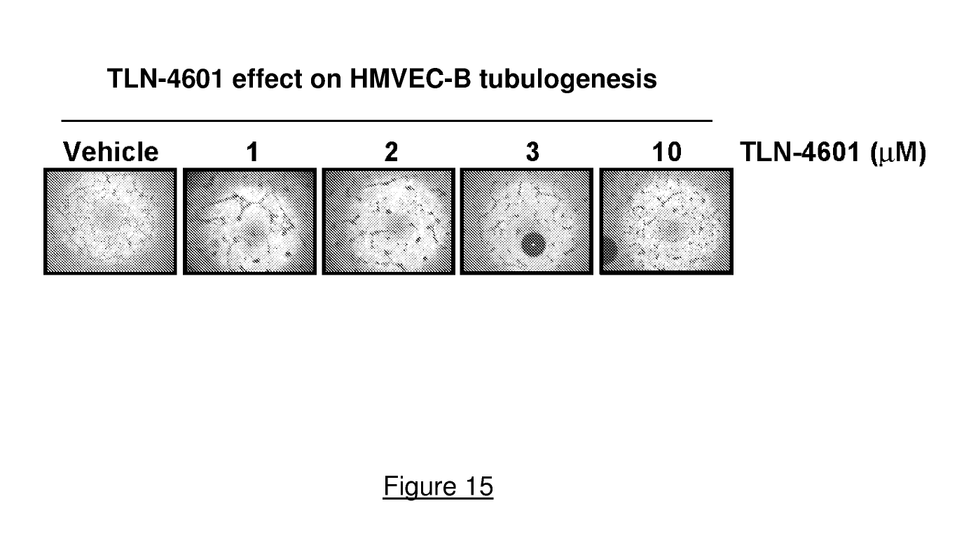

As can be seen by the results presented in FIG. 15, Compound 1 treatment of the cells resulted in a reduction of tubulogenesis, with an optimal effect observed at 10 μM.

### Example 15

### Effect of Compound 1 on S1P and LPA Mediated ERK and RAF Phosphorylation in Human Brain Endothelial Cells

Glioblastoma multiform is the most commonly occurring primary brain tumor in adults and is highly malignant, displaying increased vascularization, aggressive growth and invasion into surrounding brain tissue. Among the serum-derived lipid and growth factors that exhibit chemotactic influences towards glioblastoma cells and that induce tumor neovascularization, sphingosine-1-phosphate (S1P) is a bioactive lipid that signals through a family of five G-protein-coupled receptors termed S1PR(1-5). The S1PR contribution to intracellular calcium (Ca²⁺) homeostasis correlates with activation of extracellular signal-regulated protein kinase (ERK) MAP kinase. Interestingly, among the two sphingosine kinase (SphK) isoforms, SphK-1 correlates with short survival of glioblastoma patients, and is over-expressed in brain tumor-derived endothelial cells. Consequently, the generation of S1P is hypothesized to contribute to the acquisition and the maintenance of the multidrug resistance phenotype in brain tumors as well as to exert chemotactic migration effects in numerous types of cells including ovarian cancer cells, HT-1080 fibrosarcoma cells, U-87 glioblastoma cells and mesenchymal stromal cells. The molecular players that link the control of S1P-mediated cell migration and to extracellular matrix (ECM) degradation remain to be investigated in human brain microvascular endothelial cells (HBMEC).

The inherent signalling properties of S1P and LPA suggest, however, that both could regulate pathways involved in malignant transformation. In fact, the receptors that receive their signals are all currently investigated as potential therapeutic targets in cancer. S1P and LPA signal through a family of eight G-protein-coupled receptors, named S1P(1-5) and LPA(1-3). S1P stimulates growth and invasiveness of glioma cells, and high expression levels of the enzyme that forms S1P, sphingosine kinase-1, correlate with short survival of glioma patients.

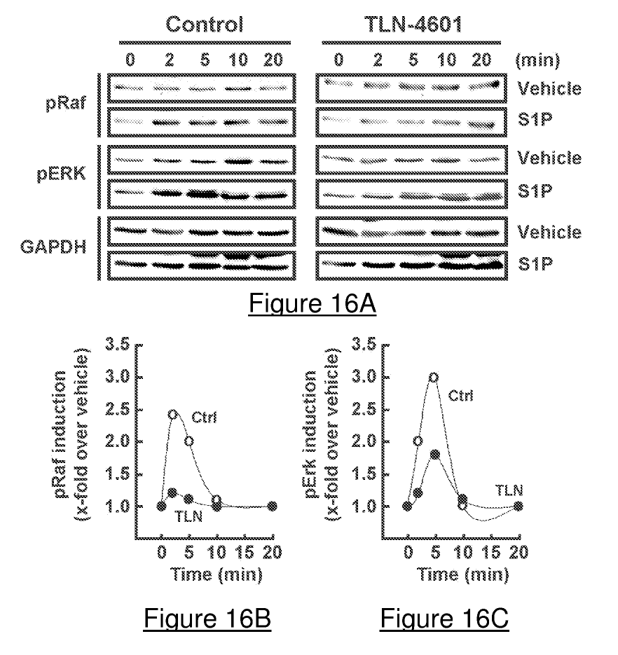

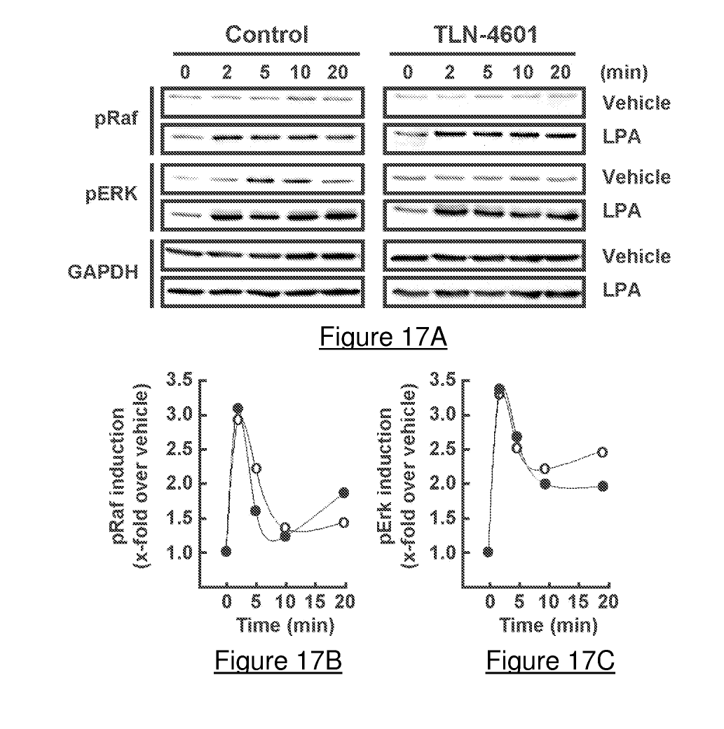

To examine the effect of Compound 1 (TLN-4601) on S1P- and LPA-mediated ERK and Raf phosphorylation in HBMEC, cells were pre-treated with TLN-4601 (5 μM for 18 hours) or vehicle and subsequently challenged by the addition of 1 μM S1P or LPA. Cell lysates were isolated at different time points until 20 minutes (FIG. 16A for S1P; FIG. 17A for LPA). Densitometric quantification shows that S1P-mediated phosphorylation of Raf and Erk was significantly reduced by TLN-4601 (FIG. 16B), while that of LPA remained unaffected (FIG. 17B).

### Example 16

### Effect of Compound 1 on a Migration of Human Brain Endothelial Cells in Response to Various Chemotactic Stimuli

Human brain microvascular endothelial cells were grown as described in Example 14.

HBMEC migration was assessed using modified Boyden chambers. The lower surfaces of Transwells (8-μm pore size; Costar, Acton, Mass.) were pre-coated with 0.2% type-I collagen for 2 hours at 37° C. The Transwells were then assembled in a 24-well plate (Fisher Scientific Ltd, Nepean, ON). The lower chamber was filled with serum-free HBMEC medium. Control HBMEC were collected by trypsinization, washed and resuspended in serum-free medium at a concentration of 10⁶ cells/ml; 10⁵ cells were then inoculated onto the upper side of each modified Boyden chamber. The plates were placed at 37° C. in 5% CO₂/95% air for 30 minutes after which various concentrations of growth factors were added to the lower chambers of the Transwells. Migration then proceeded for 6 hours at 37° C. in 5% CO₂/95% air. Cells that had migrated to the lower surfaces of the filters were fixed with 10% formalin phosphate and stained with 0.1% crystal violet-20% methanol (v/v). Images of at least five random fields per filter were digitized (100× magnification). The average number of migrating cells per field was quantified using Northern Eclipse software (Empix Imaging Inc., Mississauga, ON). Migration data are expressed as a mean value derived from at least four independent experiments.

Cell migration chemotactic response to growth factors was asessed in

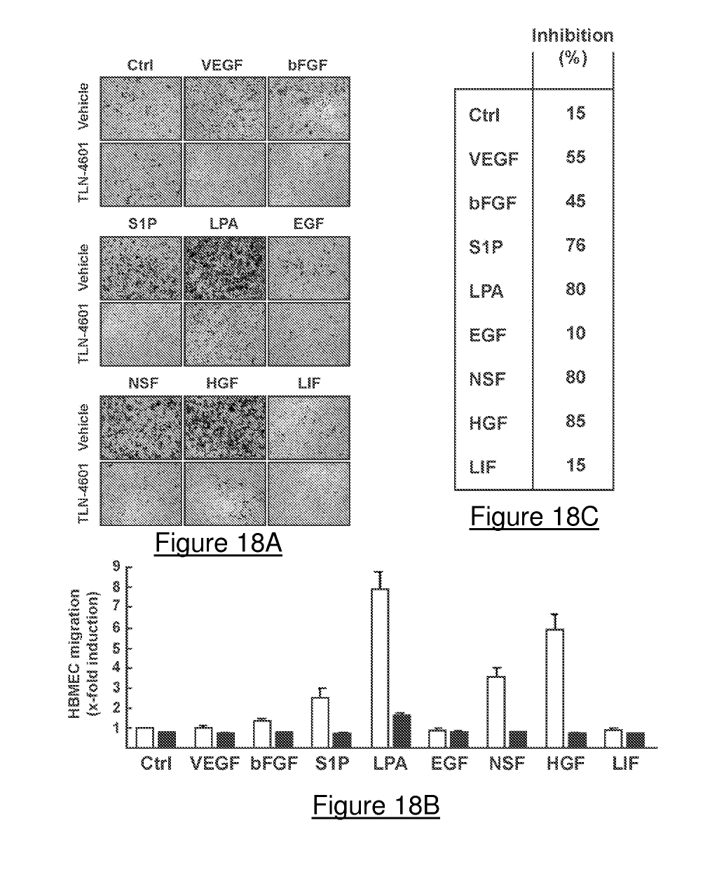

HBMEC as described above, with the results for untreated cells (FIG. 18B, white bars) and TLN-4601-treated cells (FIG. 18B, black bars) being compared to measure the effect of TLN-4601 to inhibit the migration of the endothelial cells in response to various chemotactic stimuli. A significant reduction in HBMEC migration was observed in those cells pre-treated with Compound 1 (TLN-4601) and thereafter exposed to either bFGF, VEGF, S1P, LPA, NSF, or HGF-induced migration (FIG. 18A and FIG. 18C). bFGF, basic fibroblast growth factor; EGF, epidermal growth factor; LIF, leukemia inhibitory factor; NSF, neural survival factor-1; S1P, sphingosine-1-phosphate; LPA, lysophosphatidic acid; VEGF, vascular endothelium growth factor; HGF, hepatocyte growth factor.

While this invention has been particularly shown and described with references to preferred embodiments thereof, it will be understood by those skilled in the art that various changes in form and details may be made therein without departing from the spirt of the invention.

All documents, publications, patents, books, manuals, articles, papers and other materials referenced herein are expressly incorporated herein by reference in their entireties.

1. A method of inhibiting migration of a cell, comprising contacting a cell with an effective amount of a compound of Formula I, wherein the compound of Formula I has a structure 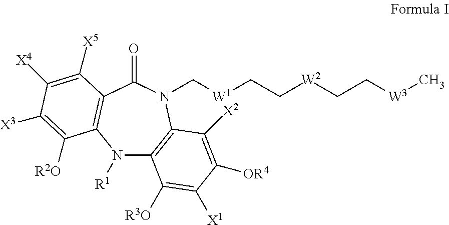
wherein,
W¹, W² and W³ are each independently 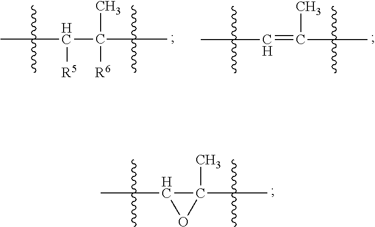
or the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;
R¹ is H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl or a C-coupled amino acid;
R², R³, and R⁴ are each independently H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl or a C-coupled amino acid;
R⁵ and R⁶ are each independently H, OH, OC₁₋₆alkyl, NH₂, NHC₁₋₆alkyl, N(C₁₋₆alkyl)₂, or NHC(O)C₁₋₆alkyl;
R⁷ is H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl or C₃₋₁₀heterocycloalkyl;
X¹, X², X³, X⁴ and X⁵ are each H; or one of X¹, X², X³, X⁴ or X⁵ is halogen and the remaining ones are H; and
wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀ heterocycloalkyl, C₆₋₁₀aryl, C₅₋₁₀heteroaryl, alkoxy, aryloxy, sulfinyl, sulfonyl, oxo, guanidino or formyl;
or a salt or an ester thereof, thereby inhibiting migration of a cell.

2. The method of claim 1, wherein the compound of Formula I is

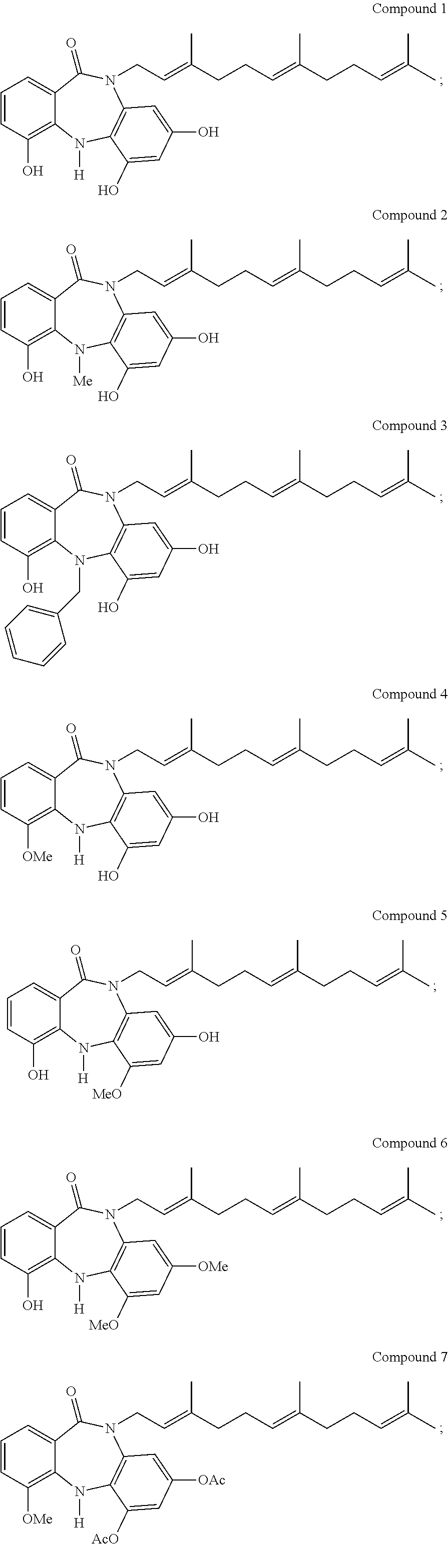

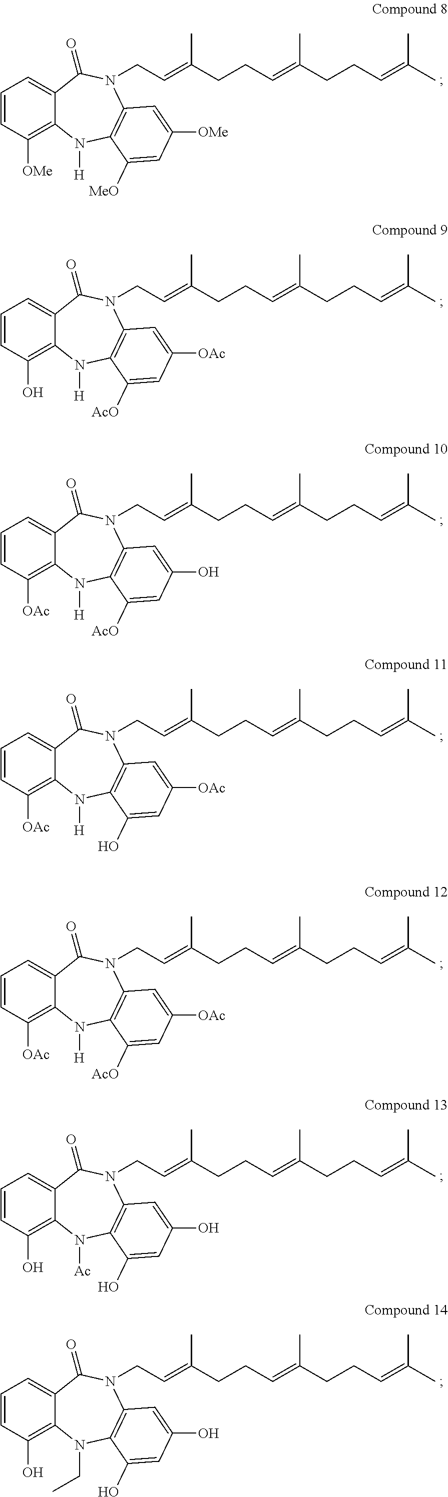

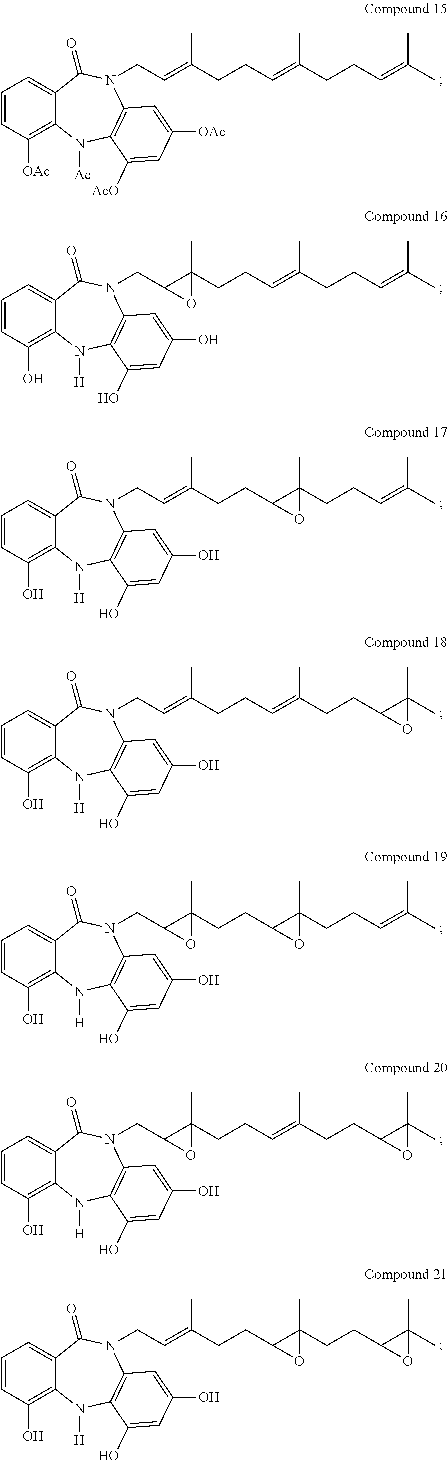

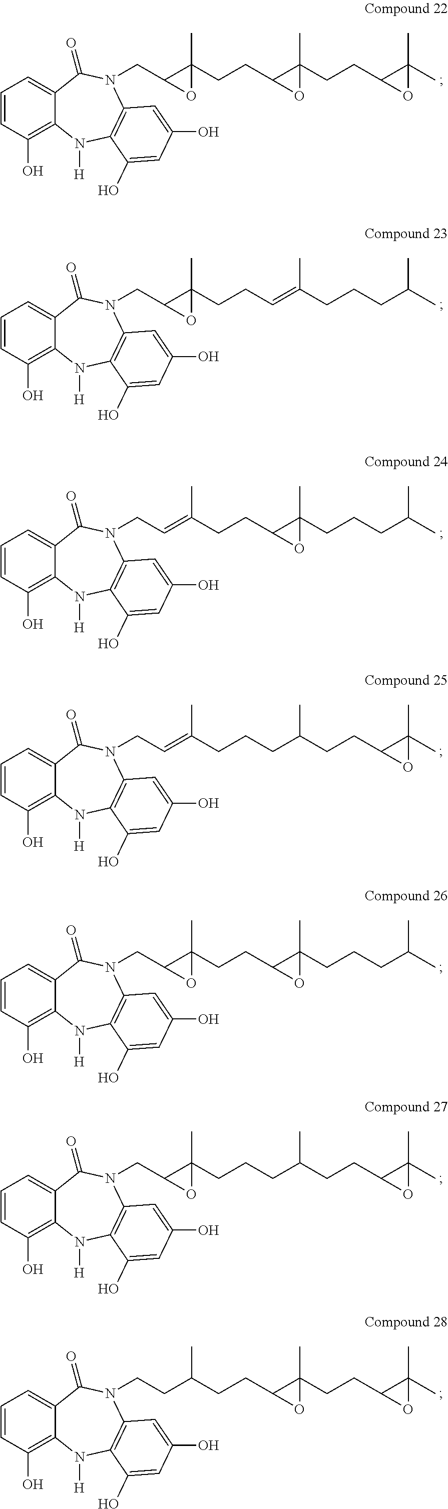

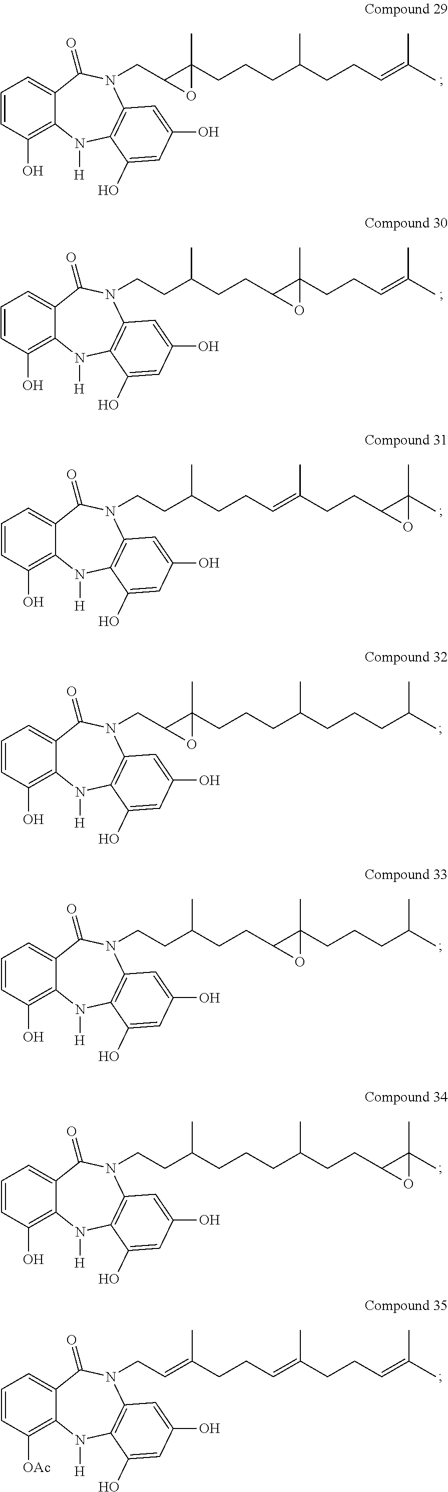

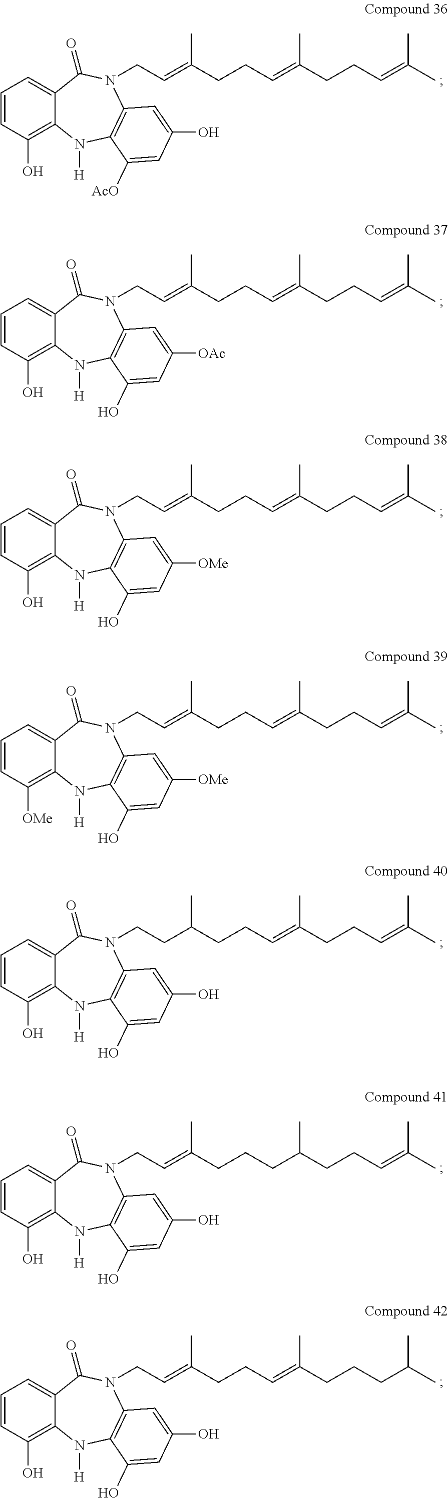

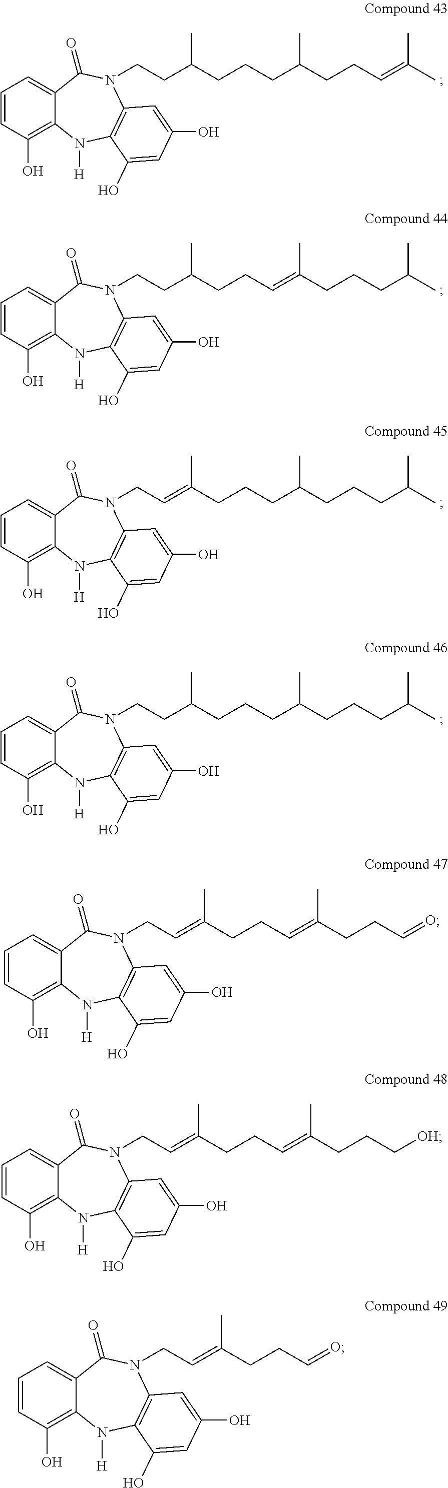

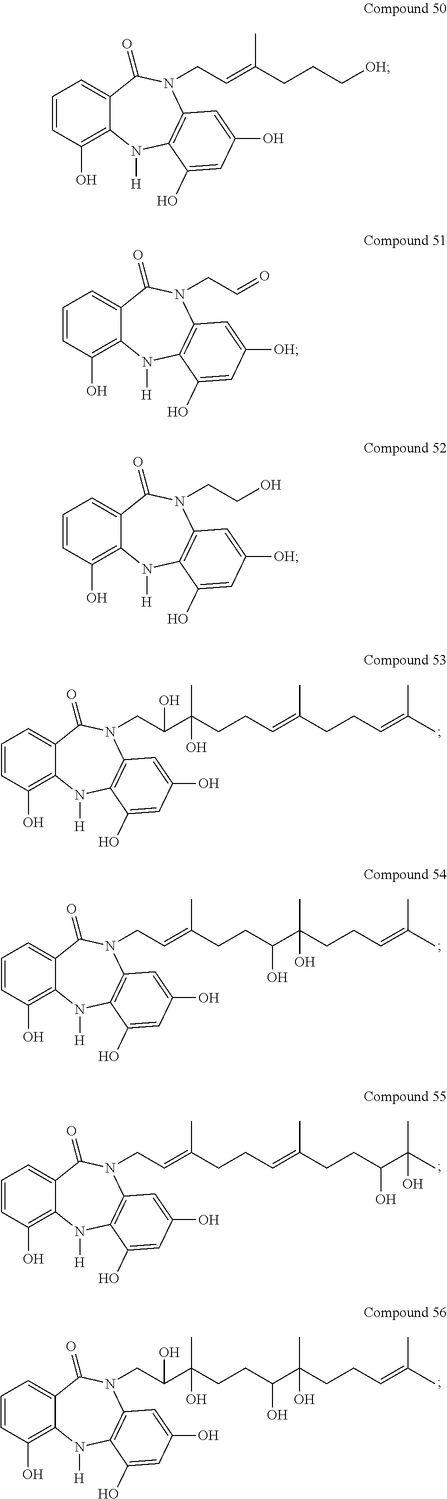

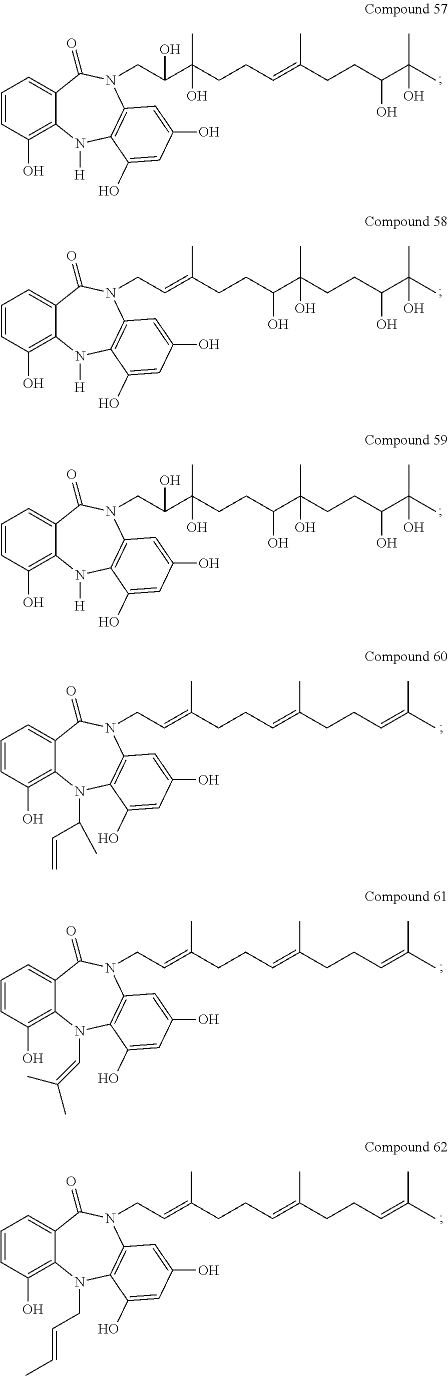

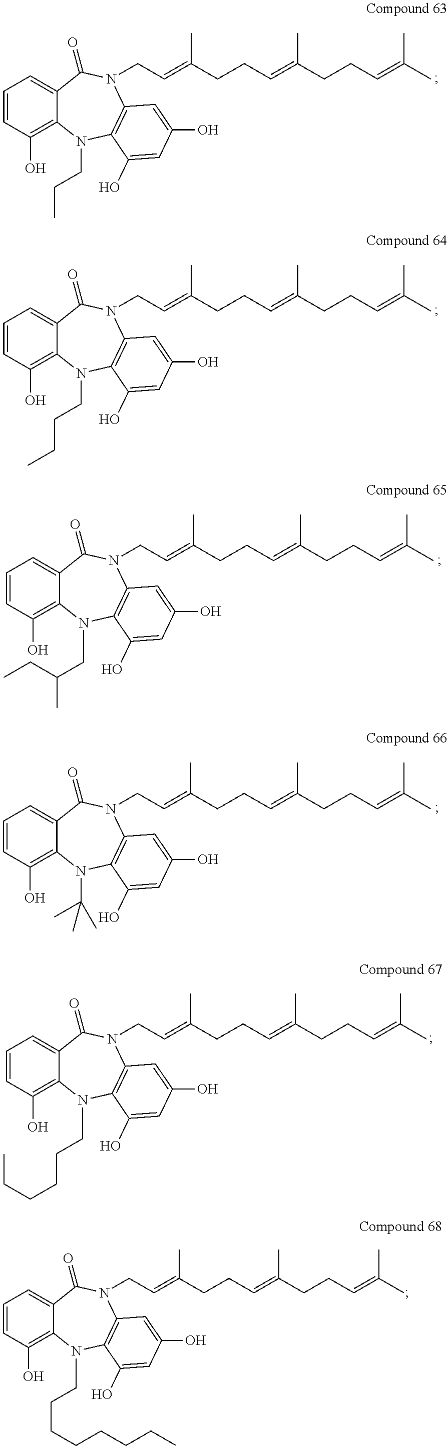

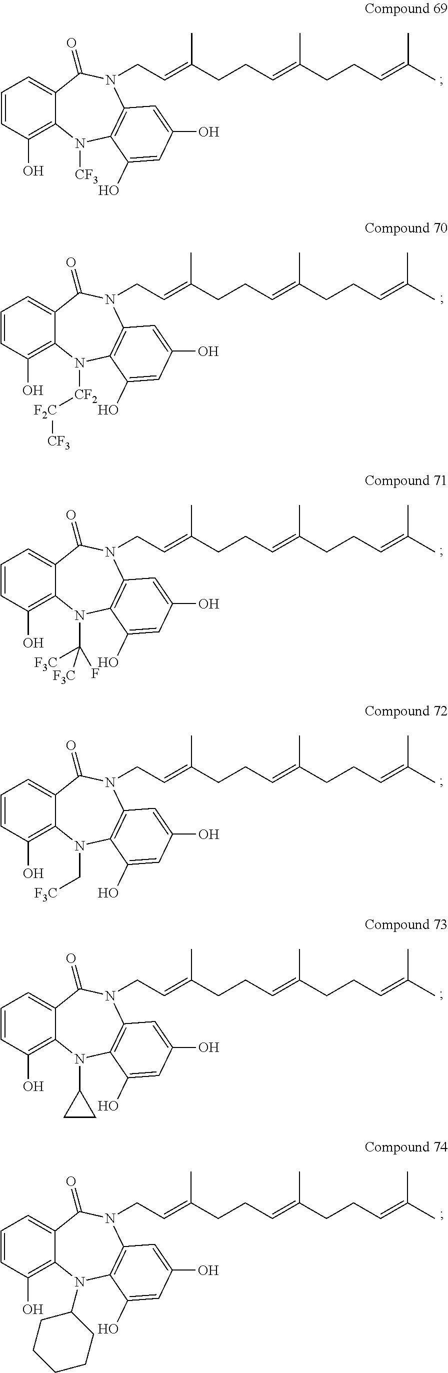

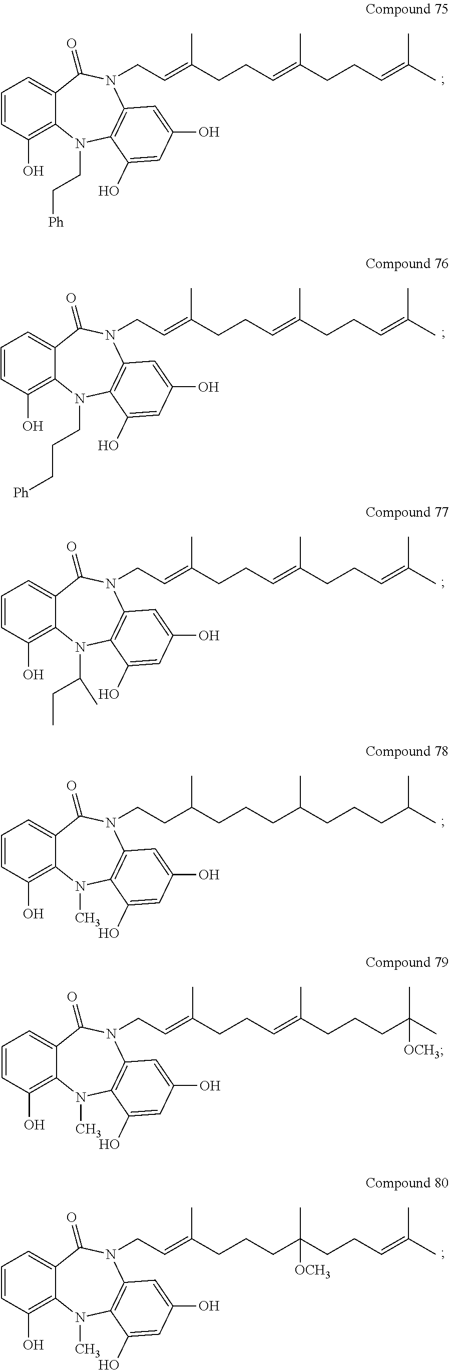

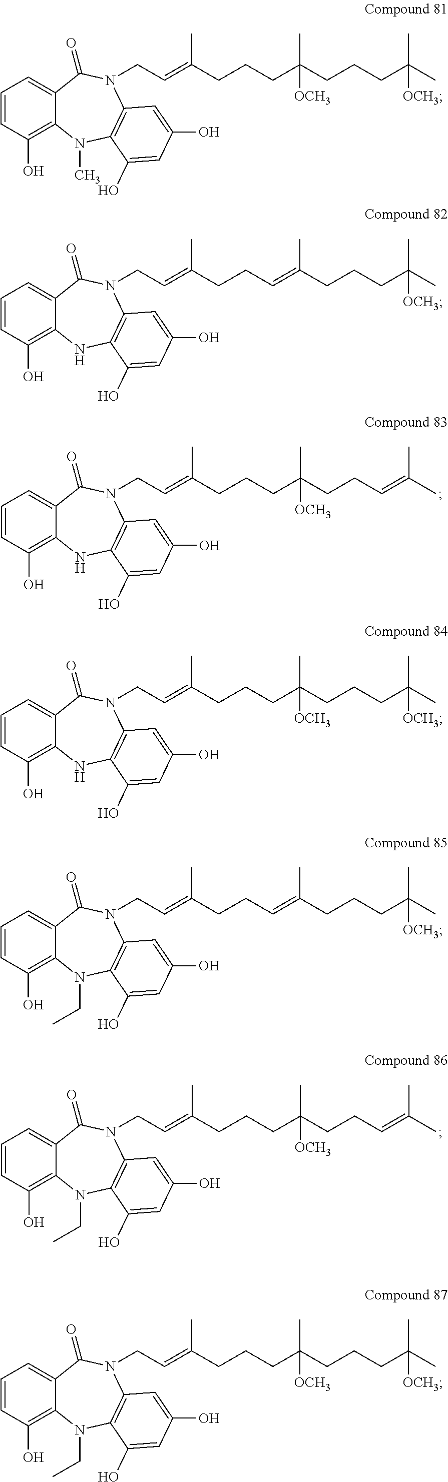

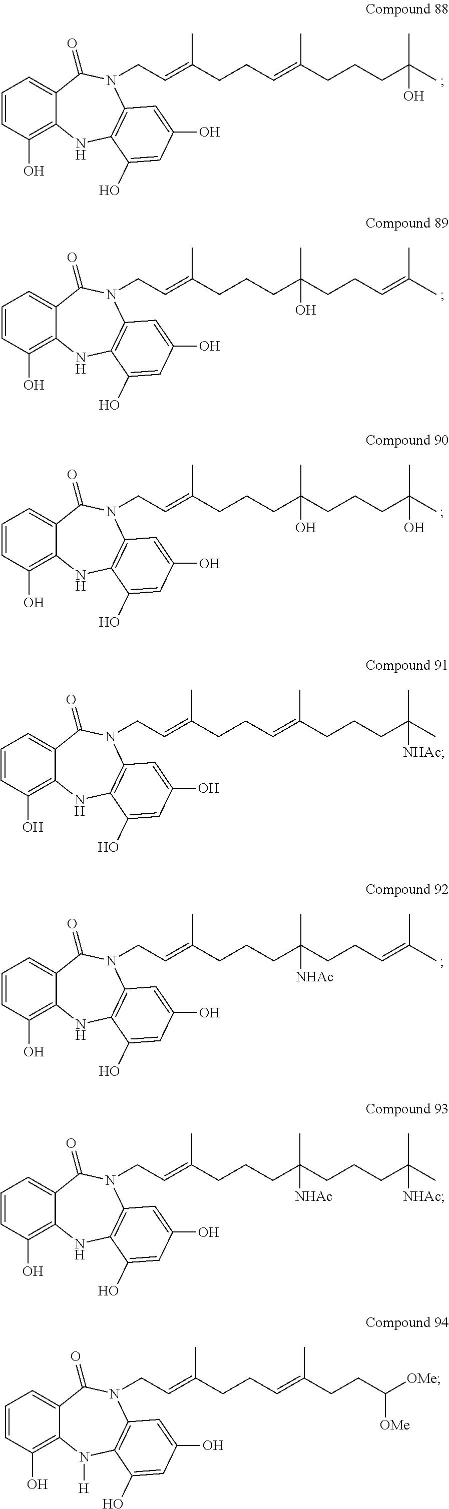

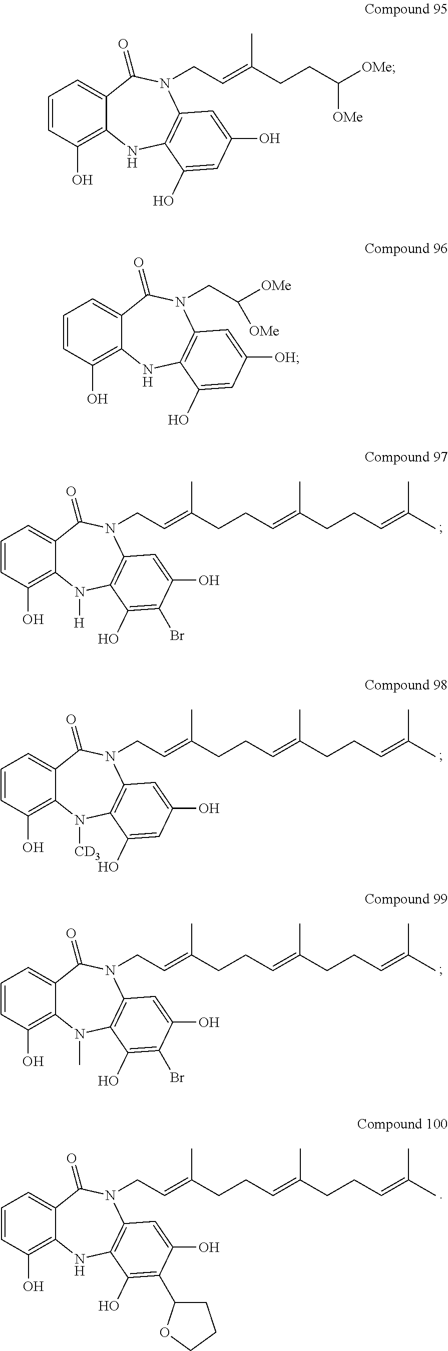

3. The method of claim 1, wherein the compound of Formula I is Compound 1 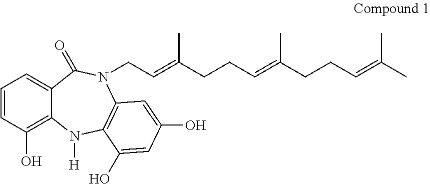
or a salt or an ester thereof.

4. The method of claim 1, wherein the cell is contacted in vitro or in vivo.

5. The method of claim 1, wherein the cell is a neoplastic cell.

6. The method of claim 1, wherein the cell is an endothelial cell.

7. The method of claim 1, wherein the migration is chemotactic migration.

8. The method of claim 7, wherein the chemotactic migration is induced by activation of a RAS-MAPK signaling pathway in the cell.

9. The method of claim 7, wherein the chemotactic migration is induced by activation of a PI3K/AKT signaling pathway in the cell.

10. The method of claim 1, wherein the cell is the cell of a breast tumor, ovarian tumor, lung tumor, non-small cell lung tumor, colon tumor, central nervous system (CNS) tumor, melanoma, renal tumor, prostrate tumor, pancreatic tumor, glioma tumor; a glioblastoma multiform tumor, or a growth factor receptor-mediated tumor.

11. The method of claim 10, wherein the cell of the glioma tumor comprises an EGF receptor mutation, a PTEN mutation, or both an EGF receptor mutation and a PTEN mutation.

12. The method of claim 11, wherein the EGF receptor mutation is an EGFRvIII mutation.

13. The method of claim 10, wherein the growth factor receptor mediated tumor is an EGF-mediated tumor.

14. A method of inhibiting migration of a cell in a subject, comprising administering an effective amount of a compound of Formula I to a subject, wherein the compound of Formula I has a structure 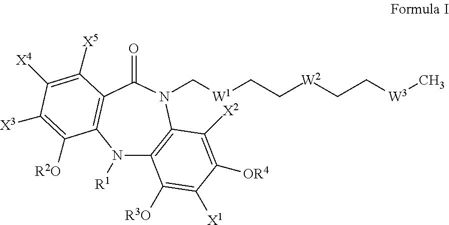
wherein,
W¹, W² and W³ are each independently 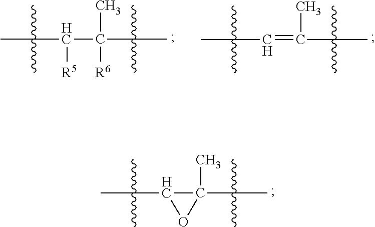
or the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;
R¹ is H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl or a C-coupled amino acid;
R², R³, and R⁴ are each independently H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl or a C-coupled amino acid;
R⁵ and R⁶ are each independently H, OH, OC₁₋₆alkyl, NH₂, NHC₁₋₆alkyl, N(C₁₋₆alkyl)₂, or NHC(O)C₁₋₆alkyl;
R⁷ is H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl or C₃₋₁₀heterocycloalkyl;
X¹, X², X³, X⁴ and X⁵ are each H; or one of X¹, X², X³, X⁴ or X⁵ is halogen and the remaining ones are H; and
wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino or formyl;
or a salt or an ester thereof, thereby inhibiting migration of a cell in a subject.

15. The method of claim 14, wherein the compound of Formula I is

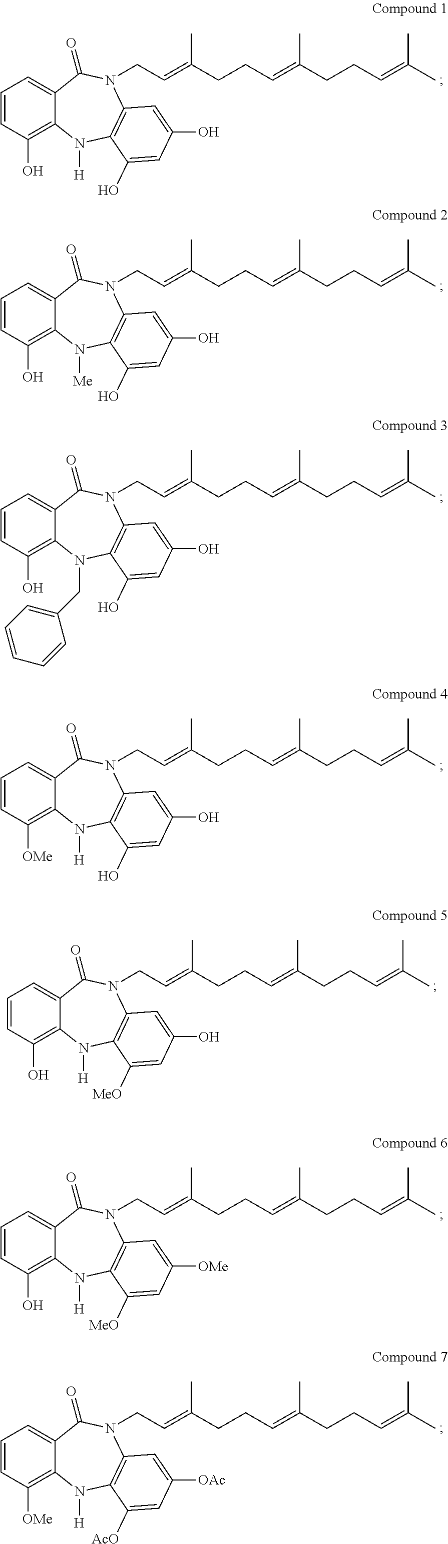

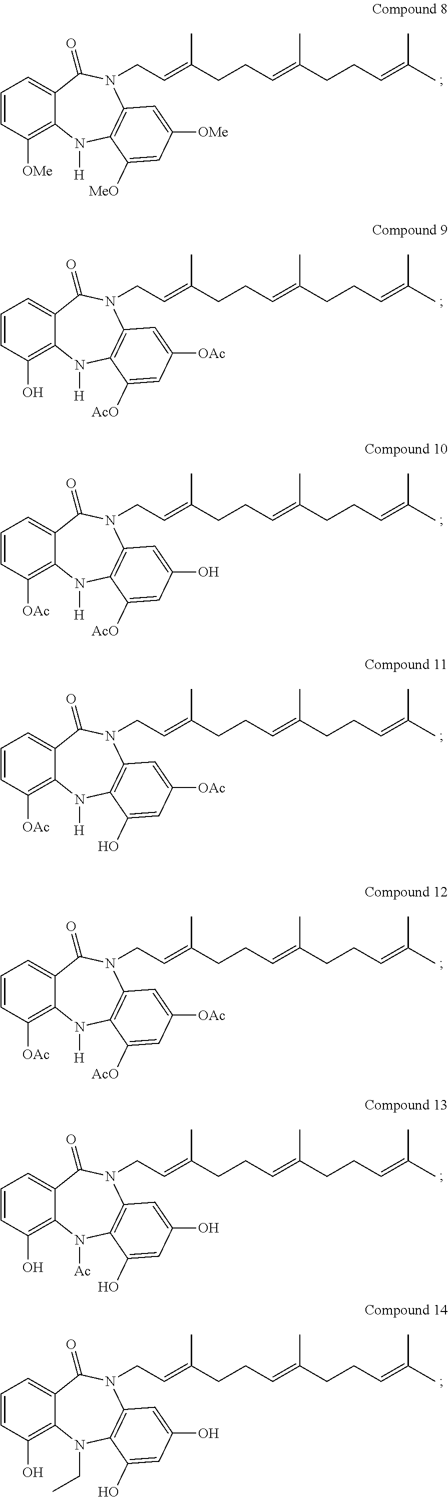

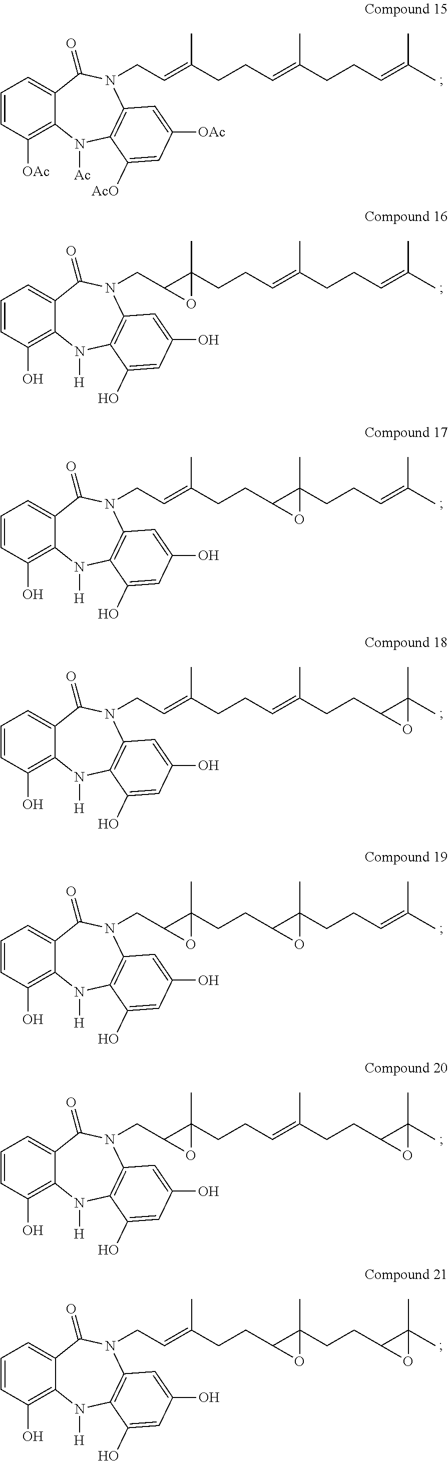

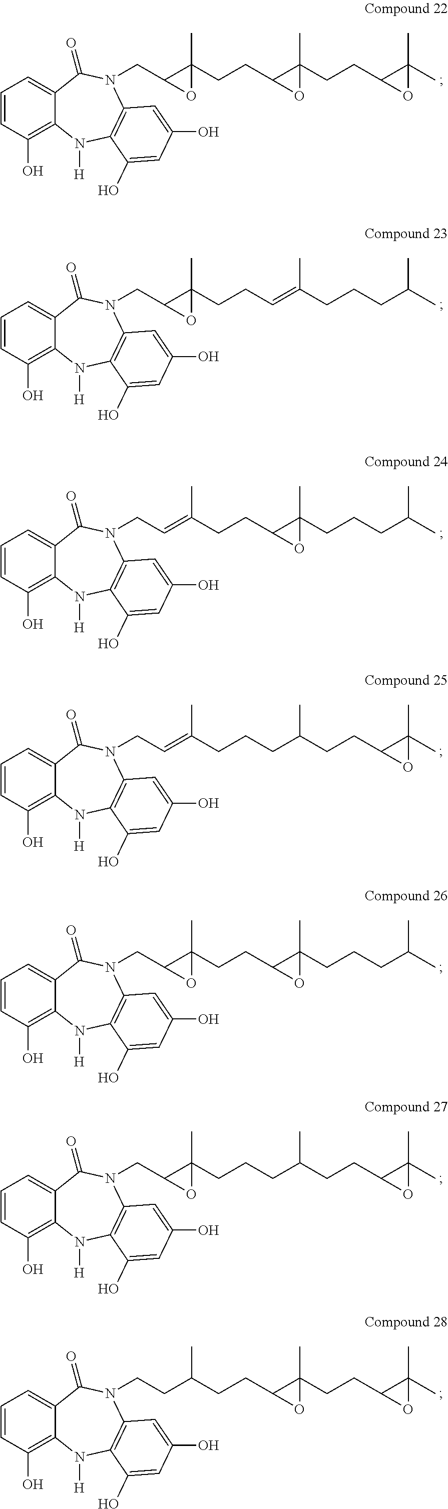

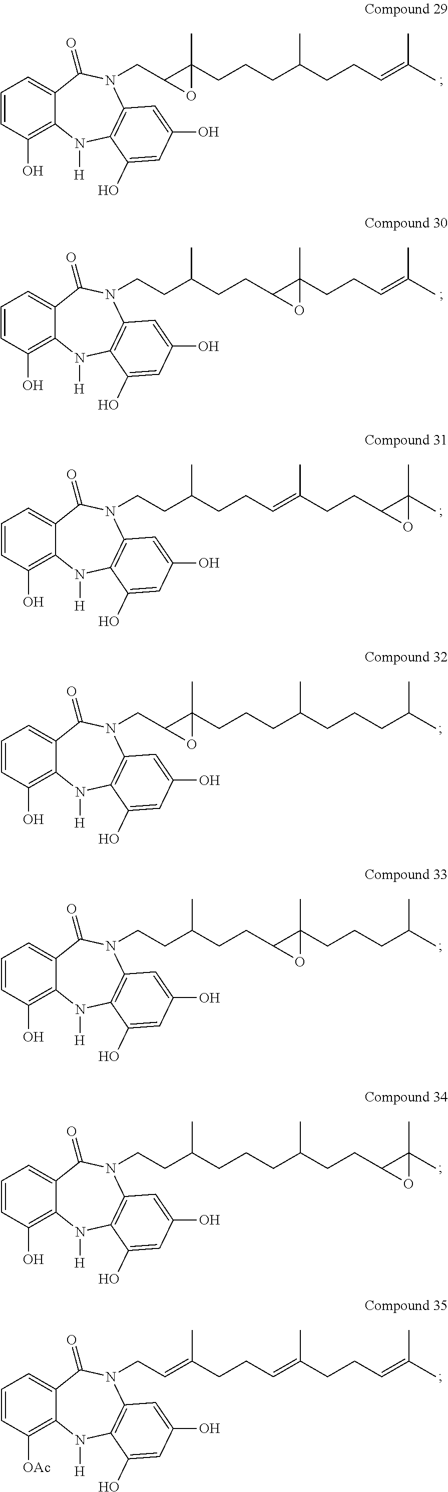

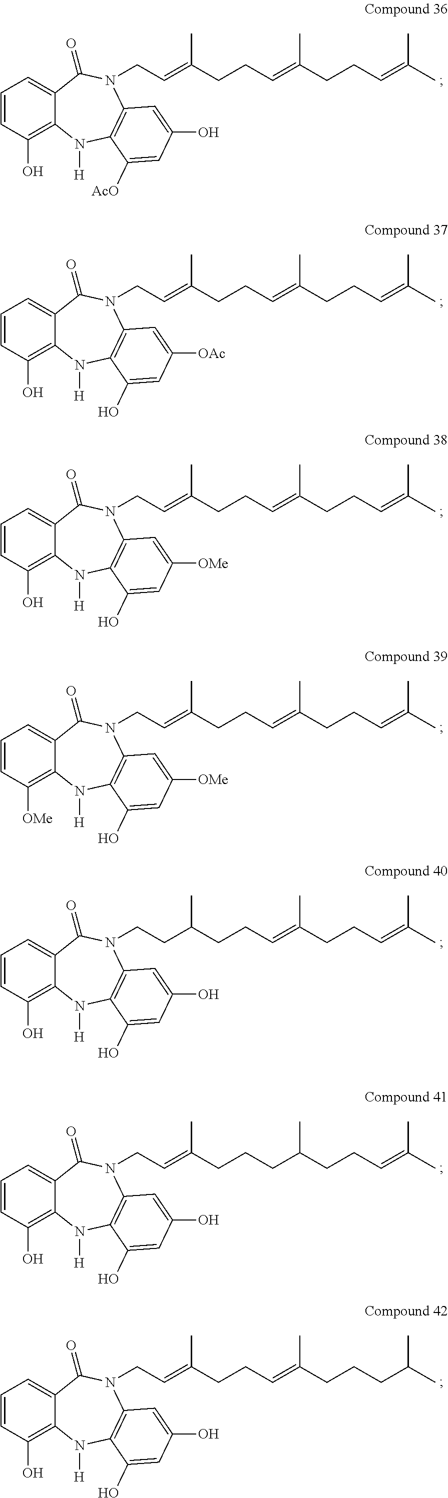

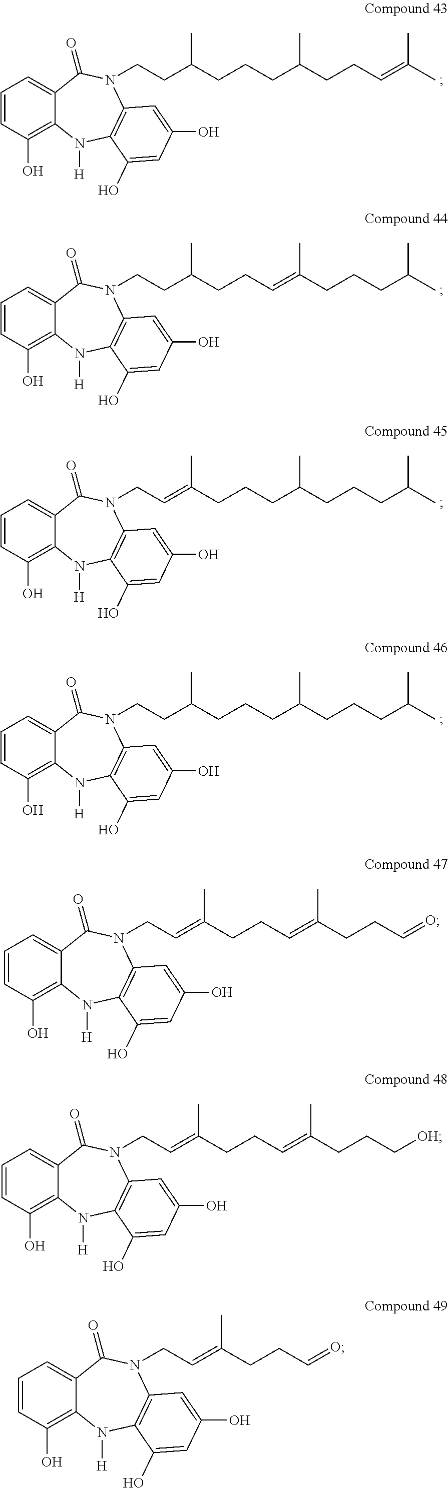

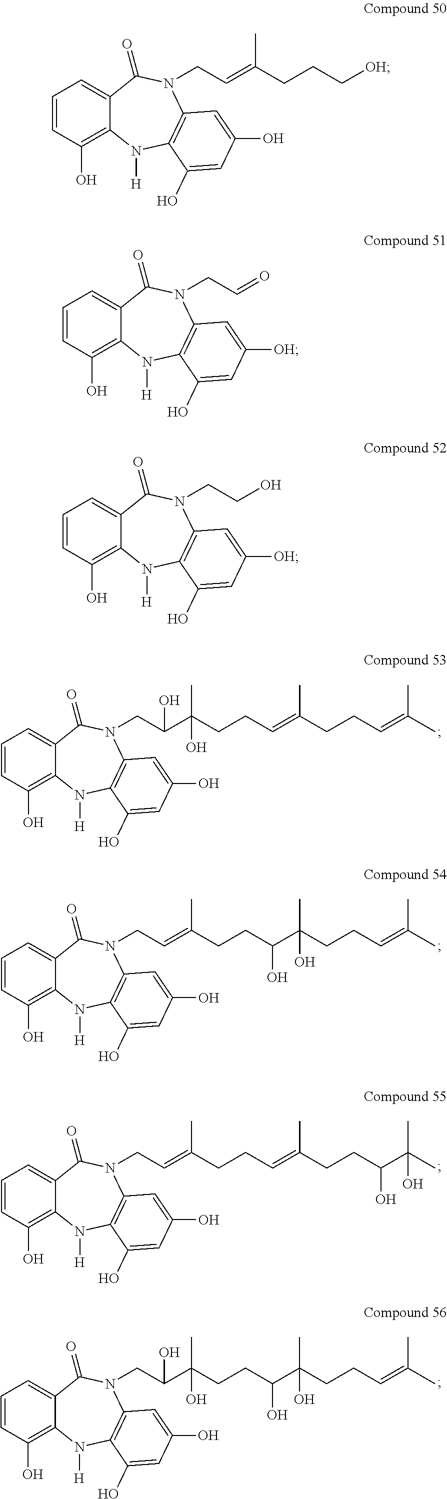

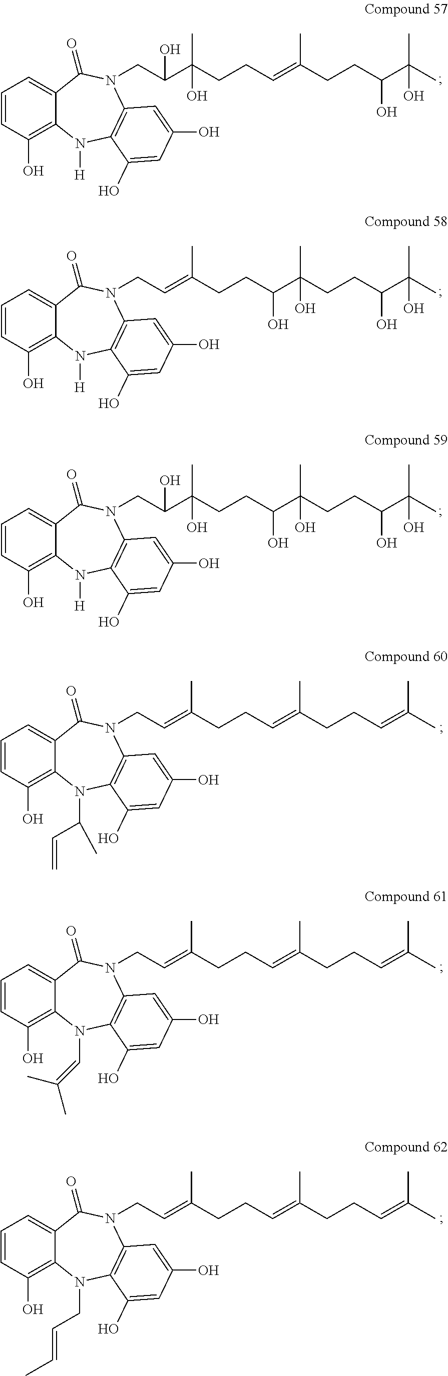

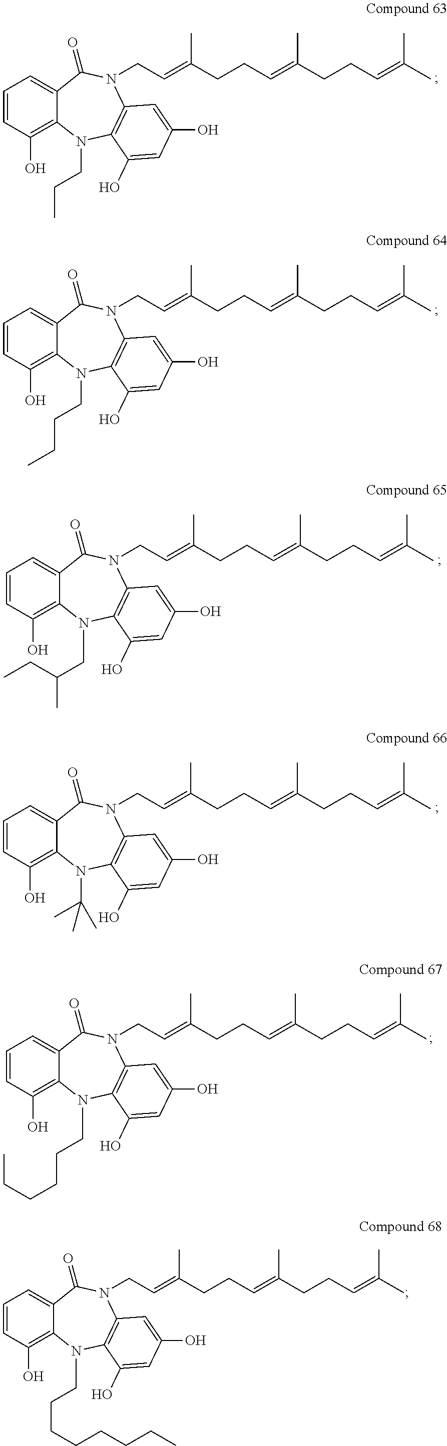

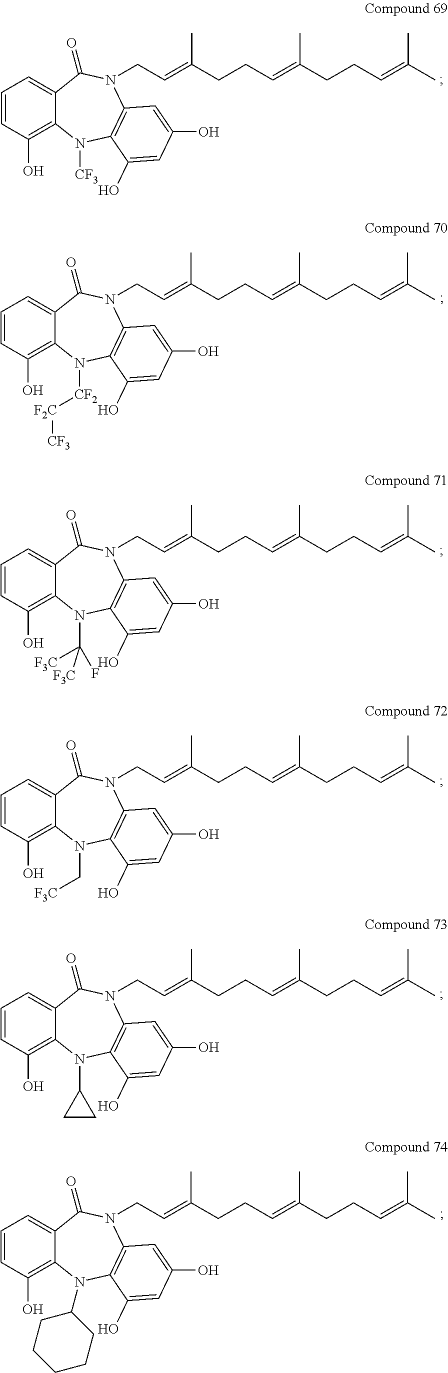

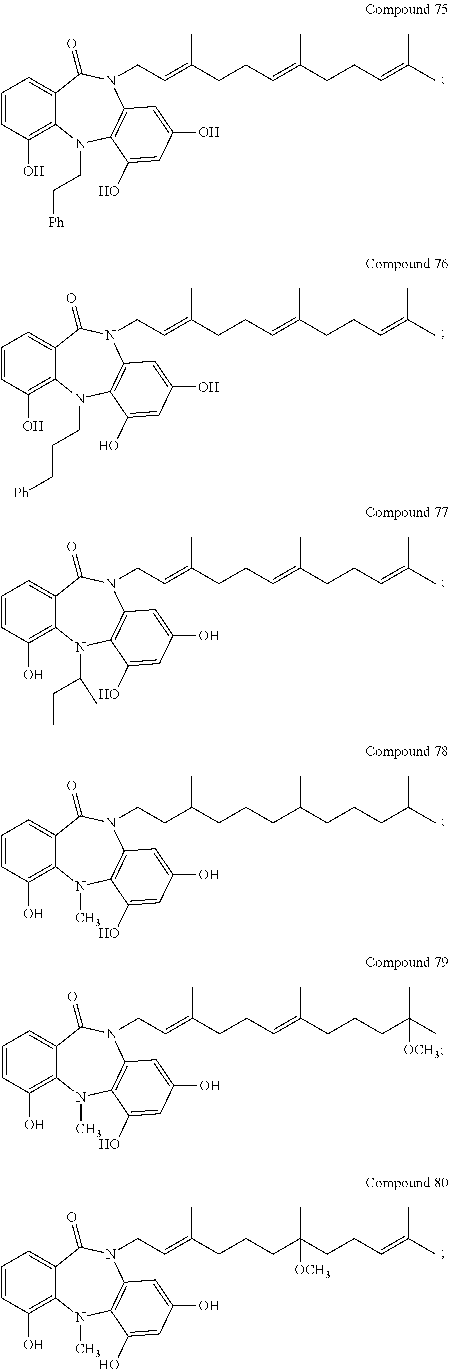

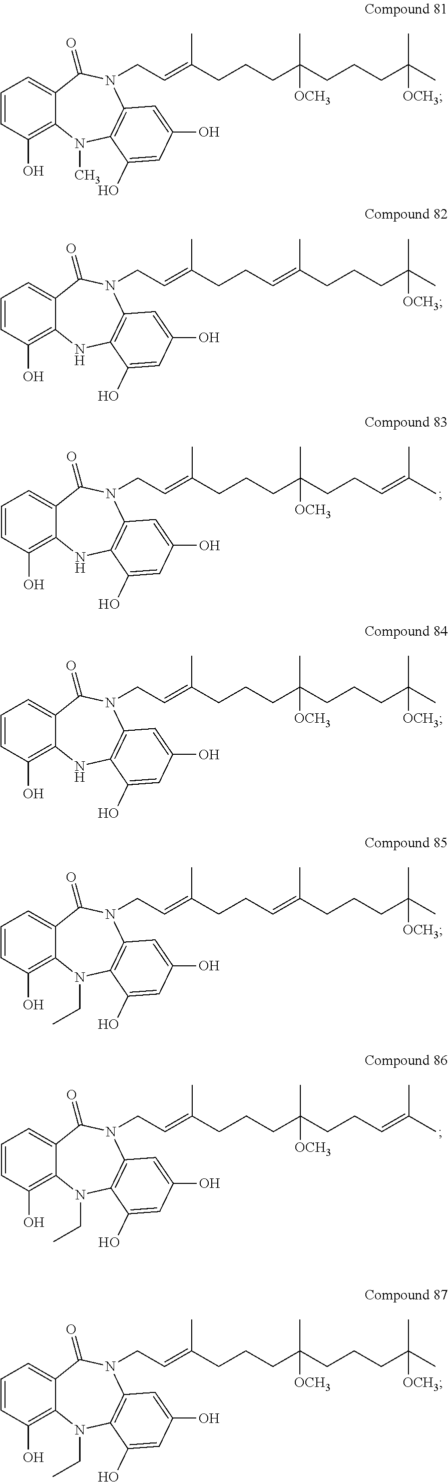

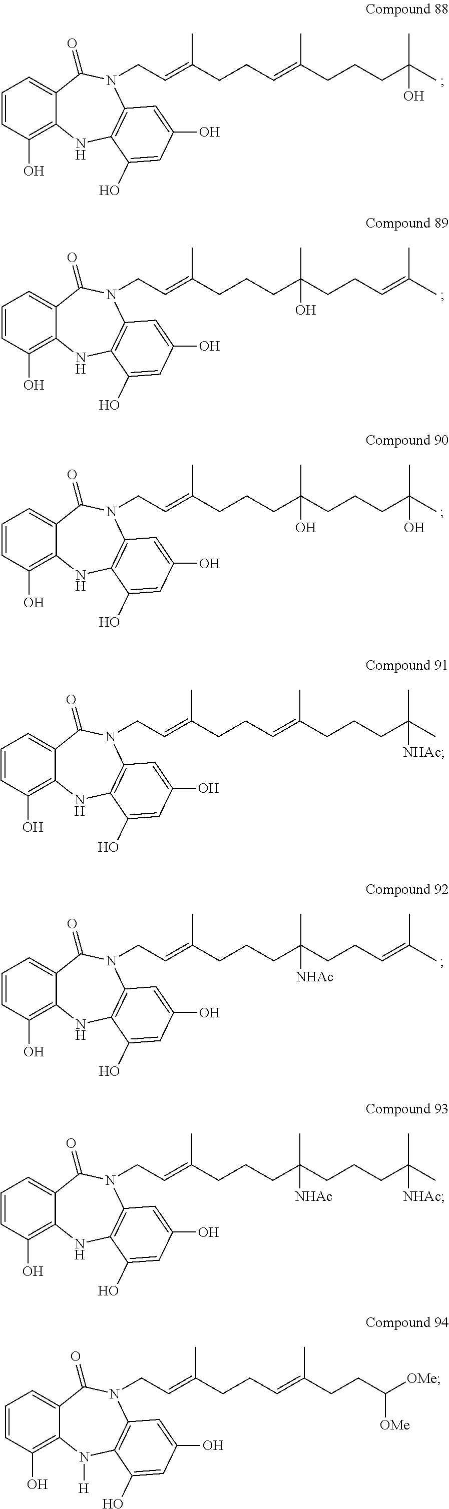

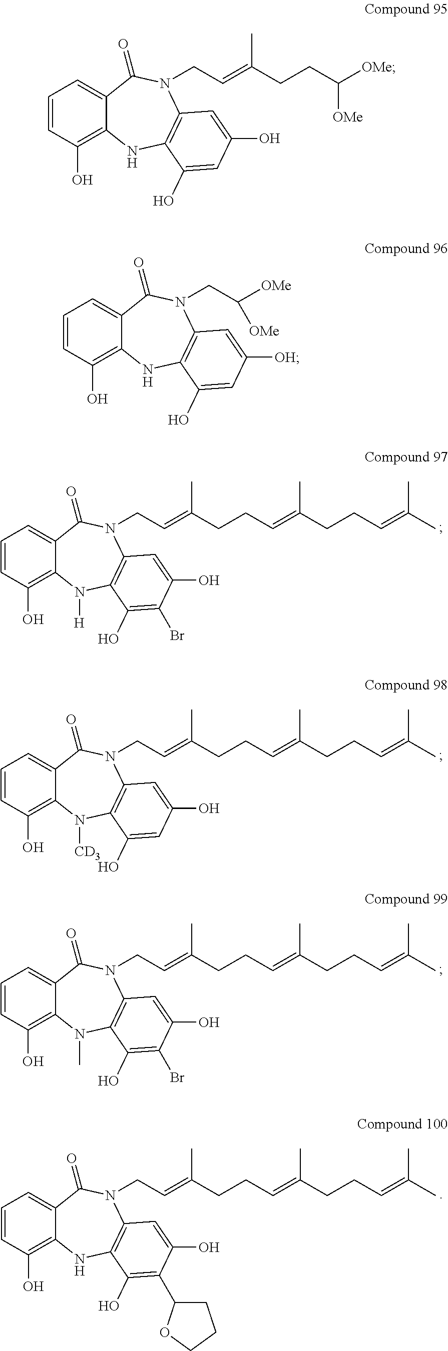

16. The method of claim 15, wherein the compound of Formula I is Compound 1 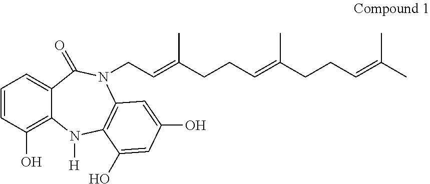
or a salt or an ester thereof.

17. The method of claim 14, wherein the compound of Formula I is administered to the subject in pharmaceutically acceptable formulation comprising the compound of Formula I and a pharmaceutically acceptable carrier.

18. The method of claim 14, wherein the cell is a neoplastic cell.

19. The method of claim 14, wherein the cell is an endothelial cell.

20. The method of claim 14, wherein the migration is chemotactic migration.

21. The method of claim 20, wherein the chemotactic migration is induced by activation of a RAS-MAPK signaling pathway in the cell.

22. The method of claim 20, wherein the chemotactic migration is induced by activation of a PI3K/AKT signaling pathway in the cell.

23. The method of claim 14, wherein the cell is the cell of a breast tumor, ovarian tumor, lung tumor, non-small cell lung tumor, colon tumor, central nervous system (CNS) tumor, melanoma, renal tumor, prostrate tumor, pancreatic tumor, glioma tumor; a glioblastoma multiform tumor; a growth factor receptor-mediated tumor, Ras-mediated tumor, or a Raf kinase-mediated tumor.

24. The method of claim 23, wherein the cell of the glioma tumor comprises an EGF receptor mutation, a PTEN mutation, or both an EGF receptor mutation and a PTEN mutation.

25. The method of claim 24, wherein the EGF receptor mutation is an EGFRvIII mutation.

26. The method of claim 23 wherein the growth factor receptor mediated tumor is an EGF-mediated tumor.
